(19) United States
(12) Patent Application Publication
(10) Pub. No.: US 2024/0139314 A1
(43) Pub. Date: May 2, 2024

(54) ENGINEERED CHIMERIC FUSION PROTEIN COMPOSITIONS AND METHODS OF USE THEREOF

(71) Applicant: Myeloid Therapeutics, Inc., Cambridge, MA (US)

(72) Inventors: Daniel GETTS, Stow, MA (US); Yuxiao WANG, Belmont, MA (US)

(21) Appl. No.: 18/486,563

(22) Filed: Oct. 13, 2023

Related U.S. Application Data
(60) Provisional application No. 63/416,182, filed on Oct. 14, 2022.

(51) Int. Cl. A61K 39/395; A61K 31/407; A61K 31/4709; A61K 31/4995; C07K 14/47; C07K 14/705; C07K 14/735; C07K 16/28; C07K 16/30; C07K 16/32; C12N 15/85

(52) U.S. Cl. CPC A61K 39/3955; A61K 31/407; A61K 31/4709; A61K 31/4995; C07K 14/47; C07K 14/70535; C07K 14/70596; C07K 16/2896; C07K 16/30; C07K 16/303; C07K 16/32; C12N 15/85; C07K 2319/03; C07K 2319/30; C07K 2319/50; C12N 2800/107

(57) ABSTRACT

The present disclosure provides compositions and methods for making and using engineered myeloid cells for immunotherapy in cancer or infection by expressing a chimeric antigen receptor having an enhanced phagocytic activity, wherein the chimeric receptor is encoded by a recombinant nucleic acid.

### CROSS REFERENCE

This application claims priority to U.S. Provisional Application No. 63/416,182, filed on Oct. 14, 2022; which is incorporated herein by reference in its entirety.

### REFERENCE TO SEQUENCE LISTING INFORMATION IN XML

This application contains a Sequence Listing which has been submitted electronically in XML format. The Sequence Listing XML is incorporated herein by reference. Said XML file, created on Nov. 14, 2023, is named 56371-741_201_SL.xml and is 234,372 bytes in size.

### BACKGROUND

Cellular immunotherapy is a promising new technology for fighting difficult to treat diseases, such as cancer, and persistent infections, and also certain diseases that are refractory to other forms of treatment. Macrophages represent the dominant cell type present in a tumor or an infection site and possess several strategic advantages such that they can be potentially utilized to treat the disease most effectively. As natural sentinels of the immune system, these cells can sense and eliminate aberrant and non-healthy cell types, including cancer cells. However, potential use of engineered macrophages for immunotherapy has not been fully explored. Specifically, activating myeloid cells in vivo and targeting them towards the infectious or disease causing cell or agent is a challenge. Newer avenues are therefore sought for using these cell types towards development of improved therapeutics, including but not limited to T cell malignancies.

### SUMMARY

The instant application for patent relates to an important finding which allows myeloid cells to be precisely targeted and specifically activated for cytotoxicity directed towards a disease causing cell or agent. The instant application also relates to generation of antigen-specific myeloid cell activation under tunable control.

In one aspect, provided herein is a composition comprising a recombinant polynucleic acid comprising a sequence encoding (A) a chimeric antigen receptor capable of specifically expressing in a myeloid cell; (B) a sequence encoding a therapeutic agent, wherein the therapeutic agent comprises a transcription factor or transcriptionally functional portion thereof, and a regulatable sequence element that can be regulated by a regulating agent; and (C) a sequence encoding the regulating agent. In some embodiments, the regulatable sequence is present in between the sequence (B), i.e., the sequence encoding the transcription factor or transcriptionally functional portion and (A), i.e., the sequence encoding the chimeric antigen receptor. In some embodiments, the sequence encoding the regulating agent (C) is separated from the sequence encoding (A) and (B) by a short auto-cleavable sequence. In some embodiments, the auto-cleavable sequence is a T2A, or a P2A sequence. In some embodiments, the chimeric fusion protein (CFP) comprises: (a) an extracellular domain comprising an antigen binding domain, (b) a transmembrane domain operatively linked to the extracellular domain, wherein the transmembrane domain is a transmembrane domain from a protein that dimerizes with Fc-gamma receptor; and (c) an intracellular domain operatively linked to the transmembrane domain. In some embodiments the therapeutic agent comprises a transcription factor or transcriptionally functional portion thereof. In some embodiments, the regulatable sequence element is a protease cleavage site disposed between the therapeutic agent and the transmembrane domain.

In some embodiments, the polynucleic acid is a single mRNA.

In one aspect, provided herein is a composition comprising a recombinant polynucleic acid, wherein the recombinant polynucleic acid comprises a sequence encoding a cell surface receptor, wherein the cell surface receptor is a chimeric fusion protein (CFP) comprising: (a) an extracellular domain comprising an antigen binding domain, (b) a transmembrane domain operatively linked to the extracellular domain; and (c) an intracellular domain operatively linked to the transmembrane domain, the intracellular domain comprising: a therapeutic agent, wherein the therapeutic agent is a transcription factor or transcriptionally functional portion thereof, and a protease cleavage site disposed between the transcription factor and the transmembrane domain.

In some embodiments, the transmembrane domain is a transmembrane domain from a protein that dimerizes with Fc-gamma receptor.

In one aspect, provided herein is a composition comprising a recombinant polynucleic acid, wherein the recombinant polynucleic acid comprises a sequence encoding a cell surface receptor, wherein the cell surface receptor is a chimeric fusion protein (CFP) comprising: (a) an extracellular domain comprising an antigen binding domain, (b) a transmembrane domain operatively linked to the extracellular domain, wherein the transmembrane domain is a transmembrane domain from a protein that dimerizes with Fc-gamma receptor; and (c) an intracellular domain operatively linked to the transmembrane domain, the intracellular domain comprising: a therapeutic agent, and a protease cleavage site disposed between the therapeutic agent and the transmembrane domain, wherein cleavage of the protease cleavage site by the protease releases the therapeutic agent from the CFP.

In some embodiments, the therapeutic agent is a transcription factor or transcriptionally functional portion thereof.

In some embodiments, the transcription factor is an inflammatory response transcription factor.

In some embodiments, the transcription factor is IRF5.

In some embodiments, the CFP lacks a protease.

In some embodiments, the intracellular domain of the CFP lacks a phosphorylated tyrosine residue.

In some embodiments, the intracellular domain of the CFP lacks a phosphotyrosine binding (PTB) domain.

In some embodiments, the protease cleavage site is a viral protease cleavage site.

In some embodiments, the viral protease cleavage site is for a viral protease derived from hepatitis C virus (HCV) nonstructural protein 3 (NS3).

In some embodiments, the viral protease cleavage site is selected from the group consisting of: an NS4A/4B junction cleavage site, an NS3/NS4A junction cleavage site, an NS4A/NS4B junction cleavage site, an NS4B/NS5A junction cleavage site, an NS5A/NS5B junction cleavage site, and variants thereof cleavable by the viral protease.

In some embodiments, the protease cleavage site is cleaved by a protease upon activation of the CFP.

In some embodiments, the protease cleavage site is cleaved by a protease upon activation of the CFP in a cell expressing Fc-gamma receptor.

In some embodiments, the CFP undergoes degradation when expressed in a cell that does not express Fc receptor γ-chain.

In some embodiments, the recombinant polynucleic acid comprises a second sequence encoding a second fusion protein.

In some embodiments, a first recombinant polynucleic acid molecule encodes the CFP and a second recombinant polynucleic acid molecule encodes the second fusion protein. In some embodiments, the second fusion protein comprises a phosphotyrosine binding domain (PTB) connected to a protease. In some embodiments, the second fusion protein is a soluble cytosolic fusion protein. In some embodiments, the protease is a soluble cytosolic protease. In some embodiments, the second fusion protein is intracellularly tethered to a cell membrane when expressed in a cell. In some embodiments, the second fusion protein is intracellularly tethered to a cell membrane via a transmembrane domain. In some embodiments, the second fusion protein comprises a dimerization domain that dimerizes with a domain of a cell surface receptor to promote association of the protease and the cell surface receptor. In some embodiments, the cell surface receptor is Fc-gamma receptor. In some embodiments, the cell surface receptor does not comprise the CFP. In some embodiments, the dimerization domain comprises a leucine zipper domain, a helix-loop-helix domain, or both. In some embodiments, the second fusion protein does not comprise a dimerization domain that promotes association of the protease with the CFP.

In some embodiments, the second fusion protein is intracellularly tethered to the cell membrane via a transmembrane domain or dimerization domain that that dimerizes with Fc receptor γ-chain.

In some embodiments, the second fusion protein is intracellularly tethered to the cell membrane via an anchor.

In some embodiments, the anchor is a glycolipid anchor.

In some embodiments, the glycolipid anchor is a glycosylphosphatidylinositol (GPI) anchor. In some embodiments, the second fusion protein lacks a cleavage site recognized by the protease. In some embodiments, the second fusion protein further comprises a degron, wherein degradation activity of the degron is inhibited by binding of the PTB domain of the second fusion protein to a phosphorylated tyrosine residue. In some embodiments, the second fusion protein further comprises a degron, wherein degradation activity of the degron is inhibited by binding of the PTB domain of the second fusion protein to a phosphorylated tyrosine residue on an endogenous receptor of a cell. In some embodiments, the endogenous receptor is not constitutively phosphorylated at the tyrosine residue. In some embodiments, the endogenous receptor is phosphorylated at the tyrosine residue in cells expressing the CFP that are bound to a diseased cell expressing an antigen recognized by the antigen binding domain of the CFP.

In some embodiments, in the composition of any one of embodiments described above, the second fusion protein further comprises a degron, wherein degradation activity of the degron is inhibited by binding of the PTB domain of the second fusion protein to a phosphorylated tyrosine residue such that the second fusion protein accumulates preferentially in cells expressing the CFP that are bound to a diseased cell expressing an antigen recognized by the antigen binding domain of the CFP. In some embodiments, the antigen binding domain is an antibody or antigen binding portion thereof.

In some embodiments, in the composition of any one of embodiments described above, the antigen binding domain comprises a Fab fragment, an scFv domain or an sdAb domain.

In some embodiments, the antigen binding domain is a CD5 binding domain, a HER2 binding domain, a GPC3 binding domain, or a TROP2 binding domain.

In some embodiments, the extracellular domain comprises an extracellular domain from CD16a, CD64, CD68 or CD89.

In some embodiments, the extracellular domain further comprises a hinge domain operatively linked to the transmembrane domain and the antigen binding domain. In some embodiments, the hinge domain is from CD8.

In some embodiments, the transmembrane domain of the CFP is a transmembrane domain from a protein that dimerizes with an endogenous Fc-gamma receptor.

In some embodiments, the transmembrane domain is a transmembrane domain from a protein that dimerizes with an endogenous Fc-gamma receptor in myeloid cells.

In some embodiments, the transmembrane domain of the CFP is a transmembrane domain from CD16a, CD64, CD68 or CD89.

In some embodiments, the intracellular domain of the CFP comprises an intracellular domain from CD16a, CD64, CD68 or CD89.

In some embodiments, the intracellular domain from CD16a, CD64, CD68 or CD89 is disposed between the transmembrane domain and the protease cleavage site.

In some embodiments, the intracellular domain of the CFP lacks an intracellular signaling domain.

In some embodiments, the intracellular domain of the CFP lacks an intracellular signaling domain disposed between the protease cleavage site and the transcription factor.

In some embodiments, the intracellular domain of the CFP lacks an intracellular signaling domain disposed C-terminal to the transcription factor.

In some embodiments, the intracellular domain of the CFP comprises an intracellular signaling domain.

In some embodiments, the intracellular domain of the CFP comprises an intracellular signaling domain disposed between the transmembrane domain and the protease cleavage site.

In some embodiments, the intracellular domain of the CFP comprises an intracellular signaling domain from FcγR, FcαR, FcεR, CD3zeta, Dectin and/or CD40.

In some embodiments, the intracellular domain of the CFP comprises a phosphoinositide 3-kinase (PI3K) recruitment domain.

In some embodiments, the PI3K recruitment domain binds a p85 regulatory subunit of PI3K.

In some embodiments, the PI3K recruitment domain is from CD19.

In some embodiments, the intracellular domain comprises two or more intracellular signaling domains.

In some embodiments, at least one of the two or more intracellular signaling domains comprises an intracellular signaling domain from CD40.

In some embodiments, the recombinant polynucleic acid is an mRNA.

In some embodiments, the composition comprises a nanoparticle delivery vehicle.

In some embodiments, the nanoparticle delivery vehicle comprises the recombinant polynucleic acid. In some embodiments, the nanoparticle delivery vehicle comprises a lipid nanoparticle.

In some embodiments, the nanoparticle delivery vehicle encapsulates the recombinant polynucleic acid. In some embodiments, the lipid nanoparticle comprises a polar lipid and a non-polar lipid. In some embodiments, the lipid nanoparticle is from 100 to 300 nm in diameter.

In some embodiments, the composition further comprises an inhibitor of the protease.

In some embodiments, the inhibitor of the protease is selected from the group consisting of: asunaprevir (ASV), danoprevir (DPV), simeprevir (SPV), grazoprevir (GPV), and any combination thereof.

In one aspect, described herein is a composition comprising an expression vector comprising the recombinant polynucleic acid of the composition of any one of embodiments described above.

In one aspect, described herein is a composition comprising the CFP encoded by the recombinant polynucleic acid of the composition of any one of embodiments described above.

In one aspect, described herein is a composition comprising a cell comprising the composition of any one of embodiments described above.

In some embodiments, the cell expresses the CFP.

In some embodiments, the cell expresses endogenous Fc-gamma receptor.

In some embodiments, the cell comprises a protease that cleaves the protease recognition domain.

In some embodiments, the cell is a mammalian cell.

In some embodiments, the cell is a human cell.

In some embodiments, the cell is a stem cell. In some embodiments, the stem cell is selected from the group consisting of: a hematopoietic stem cell (HSC), an induced pluripotent stem cell (iPSC), a mesenchymal stem cell (MSC), and a neural stem cell (NSC).

In some embodiments, the cell is an immune cell.

In some embodiments, the immune cell is selected from the group consisting of: a T cell, a B cell, a natural killer (NK) cell, a macrophage, a monocyte, a neutrophil, a dendritic cell, a mast cell, a basophil, and an eosinophil.

In some embodiments, the immune cell is a macrophage, a monocyte, or a dendritic cell.

In some embodiments, the immune cell is a CD14+ cell, a CD16+ cell or a CD14+/CD16+ cell.

In one aspect, described herein is a pharmaceutical composition comprising the composition of any one of embodiments described above.

In another aspect, described herein is a method of making the cell of the composition of any one of embodiments described herein comprising introducing the recombinant nucleic acid nucleic acid of any one of embodiments described herein into the cell.

In yet another aspect, described herein is a method of making the cell of the composition of any one of the embodiments described above comprising introducing the recombinant nucleic acid nucleic acid of any one of embodiments described herein into the cell and contacting the cell with a protease inhibitor that inhibits a protease that cleaves the protease recognition domain.

In one aspect, provided herein is a method for regulating signaling of a CFP comprising contacting the cell of the composition of any one of embodiments with a protease inhibitor that inhibits a protease that cleaves the protease recognition domain.

In one aspect, provided herein is a method for regulating transcription and/or signaling of a CFP, the method comprising halting addition of a protease inhibitor to a cell or removing a protease inhibitor from a cell, wherein the cell is the cell of the composition of any one of embodiments described above, wherein the protease inhibitor is a protease inhibitor that inhibits a protease that cleaves the protease recognition domain.

Provided herein is a method of treating a disease or condition in a human subject in need thereof, comprising administering to the human subject the composition of any one of the embodiments described herein or the pharmaceutical composition of the embodiments described herein.

In some embodiments, the disease or condition is a cancer.

In some embodiments, the cancer is a solid tumor.

In some embodiments, the cancer is a lymphoma.

In some embodiments, the cancer is T cell lymphoma.

Provided herein is a method for treating a subject in need thereof, comprising administering to the subject a pharmaceutical composition that described above. Additionally, provided herein is a method of inducing a tumor regression in a subject in need thereof, the method comprising administering intravenously to the subject a pharmaceutical composition comprising myeloid cells, wherein the myeloid cells express one or more recombinant nucleic acids encoding one or more polypeptides, and wherein at least one of the one or more polypeptides is functionally active in the tumor microenvironment, and not functionally active in a non-tumor environment.

### BRIEF DESCRIPTION OF DRAWINGS

FIG. 1 is a graphical representation of design of antigen specific, myeloid specific activation of pro-inflammatory transcription factor with tunable control. Left, a graphical representation of a polynucleic acid construct in a nanoparticle (LNP). The exemplified mRNA construct is magnified and the component sequences are shown below in the 5′-3′ orientation showing the CAR construct with intracellular domain encoding a transcription factor: G, guanidine; P, Phosphate; scFv, exemplary sequence for a binder domain single chain variable fragment; CD89, transmembrane domain from CD89; NS3-TF, exemplary intracellular domain comprising an NS3 protease sensitive domain and TF, transcription factor; T2A, auto-cleavable sequence; SH2-NS3p, SH2 domain fused with NS3 protease; AAA, poly A sequence. Right, a graphical representation of a chimeric antigen receptor (CAR) construct with intracellular TF domain, for expression in a myeloid cell that expresses on the membrane by multimerizing with an endogenous Fcγ-chain, and comprises an NS3 protease recognition and cleavage site connecting between the intracellular transcription factor (TF) domain (for example, an IRF5 transcription factor). The SH2-NS3p protein (having a Syk SH2-domain fused to a transcription factor) translated from the construct and cleaved at the auto-cleavable T2A domain is also shown.

FIG. 2 shows the graphical representation as described above, and the use of a protease inhibitor used as a control or regulator of the SH2-NS3p function. As long as the protease inhibitor is administered to block the function of the SH2-NS3p protease, the protease does not cleave the NS3 cleavage site, and the transcription factor is not released. Blockage of the protease with use of protease inhibitor keeps the TF in its repressed mode.

FIG. 3 is a graphical illustration showing expression of the CD89-NS3-TF construct on the cell surface when taken up by a cell naturally expressing an endogenous Fcγ-chain. The chimeric construct in a LNP is shown in the left hand side figure. The CD89 domain helps multimerization of the construct with the endogenous Fcγ-chain (middle). In absence of endogenous Fcγ-chain, i.e., when the construct is taken up by a cell that does not express the endogenous Fcγ-chain, the chimeric protein is not expressed on the cell surface, and is degraded (right). An endogenous Fcγ-chain is predominantly expressed in a myeloid cell.

FIG. 4A shows a graphical illustration of the mechanism of action of an antigen specific, myeloid cell specific activation of pro-inflammatory transcription factor with tunable control described in FIGS. 1-3. FIG. 4A (left) shows the construct design identical to FIG. 1. FIG. 4A (right) shows the target specific binding of the extracellular binding domain of the CAR, as shown to be bound to the target antigen on an exemplary target cell (a cancer cell), which then activates the chimeric receptor in the multimeric complex, and thereby phosphorylates the Fcγ-chain intracellular domain. Next, the coexpressed Syk protein fragment with the SH2 domain of the Syk-SH2-NS3 fusion protein binds to the phosphorylated residues on the Fcγ-chain, and thereby brings the protease in proximity to the NS3 cleavage site in the multimer complex.

FIG. 4B is an illustration showing that the action following the binding of the Syk-SH2-NS3 fusion protein to the phosphorylated residues in the CAR-Fcγ-chain multimer complex cleaves the NS3 cleavage site and releases the transcription factor, which localizes to the nucleus and induces transcription. For example, for an exemplary TF, IRF5, the genes under control of the TF are induces, e.g., IRF5 responsive genes, such as viral response genes and pro-inflammatory genes.

### DETAILED DESCRIPTION

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure can be described in the context of a single embodiment, the features can also be provided separately or in any suitable combination. Conversely, although the present disclosure can be described herein in the context of separate embodiments for clarity, the disclosure can also be implemented in a single embodiment.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the disclosure, and vice versa. Furthermore, compositions of the disclosure can be used to achieve methods of the disclosure.

In some embodiments, the term “about” or “approximately” as used herein when referring to a measurable value such as a parameter, an amount, a temporal duration, and the like, is meant to encompass variations of +/−20% or less, +/−10% or less, +/−5% or less, or +1-1% or less of and from the specified value, insofar such variations are appropriate to perform in the present disclosure. It is to be understood that the value to which the modifier “about” or “approximately” refers is itself also specifically disclosed.

A chimeric antigen receptor (CAR) may be interchangeably used herein with a phagocytic receptor fusion protein (PFP). A phagocytic receptor (PR) may mean a phagocytic scavenger receptor (PSR) as described herein. An “agent” may include any type of molecule and includes, but is not limited to, an antibody, a peptide, a protein, a polynucleotide (e.g., an oligonucleotide, RNA, or DNA), a small molecule, derivatives thereof and analogs thereof.

A “biologic sample” is any tissue, cell, fluid, or other material derived from an organism. As used herein, in some embodiments, the term “sample” may include a biologic sample such as any tissue, cell, fluid, or other material derived from an organism.

“Specifically binds” may refer to a compound (e.g., peptide) that recognizes and binds a molecule (e.g., polypeptide), but does not substantially recognize and bind other molecules in a sample, for example, a biological sample.

In some embodiments, the term “immune response” may include T cell mediated and/or B cell mediated immune responses that are influenced by modulation of T cell costimulation. Exemplary immune responses include T cell responses, e.g., cytokine production, and cellular cytotoxicity. In addition, In some embodiments, the term immune response includes immune responses that are indirectly affected by T cell activation, e.g., antibody production (humoral responses) and activation of cytokine responsive cells, e.g., macrophages.

A “functional derivative” of a native sequence polypeptide may be a compound having a qualitative biological property in common with a native sequence polypeptide. “Functional derivatives” may include, but are not limited to, fragments of a native sequence and derivatives of a native sequence polypeptide and its fragments, provided that they have a biological activity in common with a corresponding native sequence polypeptide. In some embodiments, the term “derivative” encompasses both amino acid sequence variants of polypeptide and covalent modifications thereof.

The terms “phagocytic cells” and “phagocytes” may be used interchangeably herein, for example, to refer to a cell that is capable of phagocytosis. There are three main categories of phagocytes: macrophages, mononuclear cells (histiocytes and monocytes); polymorphonuclear leukocytes (neutrophils) and dendritic cells.

In some cases, the term “biological sample” may encompass a variety of sample types obtained from an organism and can be used in a diagnostic or monitoring assay. In some embodiments, the term encompasses blood and other liquid samples of biological origin, solid tissue samples, such as a biopsy specimen or tissue cultures or cells derived therefrom and the progeny thereof. In some cases, the term encompasses samples that have been manipulated in any way after their procurement, such as by treatment with reagents, solubilization, or enrichment for certain components. In some cases, the term encompasses a clinical sample, and also includes cells in cell culture, cell supernatants, cell lysates, serum, plasma, biological fluids, and tissue samples.

As used herein, in some embodiments, the term “antigen-presenting cell” or “antigen-presenting cells” or its abbreviation “APC” or “APCs” may refer to a cell or cells capable of endocytosis adsorption, processing and presenting of an antigen. In some embodiments, the term may include professional antigen presenting cells for example; B lymphocytes, monocytes, dendritic cells (DCs) and Langerhans cells, as well as other antigen presenting cells such as keratinocytes, endothelial cells, glial cells, fibroblasts and oligodendrocytes. In some embodiments, the term “antigen presenting” may mean the display of antigen as peptide fragments bound to MHC molecules, on the cell surface. Many different kinds of cells may function as APCs including, for example, macrophages, B cells, follicular dendritic cells and dendritic cells. APCs can also cross-present peptide antigens by processing exogenous antigens and presenting the processed antigens on class I MHC molecules. Antigens that give rise to proteins that are recognized in association with class I MHC molecules are generally proteins that are produced within the cells, and these antigens are processed and associate with class I MHC molecules.

A phagocytic cell of the present disclosure may be an engineered phagocyte, that expresses a recombinant nucleic acid encoding a chimeric fusion protein that binds to an antigen or an epitope on a cancer cell and the engineered cell may be capable of readily engulfing the cancer cell to remove it from the body.

A chimeric fusion protein, in some cases, may refer to a chimeric receptor molecule, generated by recombinant nucleic acid technology. In some cases, a chimeric fusion protein may comprise a transmembrane protein generated by recombinant technology, and the chimeric fusion protein may comprise at least a transmembrane domain. In some cases, the chimeric fusion protein may comprise an extracellular domain and one or more intracellular domain,

An “epitope” may refer to a portion of an antigen or other macromolecule capable of forming a binding interaction with the variable region binding pocket of an antibody or TCR. In some embodiments, the term may include any protein determinant capable of specific binding to an antibody, antibody peptide, and/or antibody-like molecule (including but not limited to a T cell receptor) as defined herein. Epitopic determinants typically consist of chemically active surface groups of molecules such as amino acids or sugar side chains and generally have specific three-dimensional structural characteristics as well as specific charge characteristics. A “T cell epitope” may refer to a peptide sequence which can be bound by the MHC molecules of class I or II in the form of a peptide-presenting MHC molecule or MHC complex and then, in this form, be recognized and bound by cytotoxic T-lymphocytes or T-helper cells, respectively.

In some embodiments, the term “antibody” or “antibody moiety” may include any polypeptide chain-containing molecular structure with a specific shape that fits to and recognizes an epitope, where one or more non-covalent binding interactions stabilize the complex between the molecular structure and the epitope. Antibodies utilized in the present invention may be polyclonal antibodies, although monoclonal antibodies are preferred because they may be reproduced by cell culture or recombinantly, and can be modified to reduce their antigenicity. In some embodiments, the term may include IgG (including IgGl, IgG2, IgG3, and IgG4), IgA (including IgA1 and IgA2), IgD, IgE, IgM, and IgY, and may include whole antibodies, including single-chain whole antibodies, and antigen-binding (Fab) fragments thereof. Antigen-binding antibody fragments may include, but are not limited to, Fab, Fab′ and F(ab′)2, Fd (consisting of VH and CH1), single-chain variable fragment (scFv), single-chain antibodies, disulfide-linked variable fragment (dsFv) and fragments comprising either a VL or VH domain. The antibodies may be from any animal origin. Antigen-binding antibody fragments, including single-chain antibodies, may comprise the variable region(s) alone or in combination with the entire or partial of the following: hinge region, CH1, CH2, and CH3 domains. Also included are any combinations of variable region(s) and hinge region, CH1, CH2, and CH3 domains. Antibodies may be monoclonal, polyclonal, chimeric, humanized, and human monoclonal and polyclonal antibodies which, e.g., specifically bind an HLA-associated polypeptide or an HLA-peptide complex. A person of skill in the art will recognize that a variety of immunoaffinity techniques are suitable to enrich soluble proteins, such as soluble HLA-peptide complexes or membrane bound HLA-associated polypeptides, e.g., which have been proteolytically cleaved from the membrane. These may include techniques in which (1) one or more antibodies capable of specifically binding to the soluble protein are immobilized to a fixed or mobile substrate (e.g., plastic wells or resin, latex or paramagnetic beads), and (2) a solution containing the soluble protein from a biological sample is passed over the antibody coated substrate, allowing the soluble protein to bind to the antibodies. The substrate with the antibody and bound soluble protein is separated from the solution, and optionally the antibody and soluble protein are disassociated, for example by varying the pH and/or the ionic strength and/or ionic composition of the solution bathing the antibodies. Alternatively, immunoprecipitation techniques in which the antibody and soluble protein are combined and allowed to form macromolecular aggregates can be used. The macromolecular aggregates can be separated from the solution by size exclusion techniques or by centrifugation.

In some cases, a polypeptide used herein may be a “protein”, including but not limited to a glycoprotein, a lipoprotein, a cellular protein or a membrane protein. A polypeptide may comprise one or more subunits of a protein. A polypeptide may be encoded by a recombinant nucleic acid. In some cases, polypeptide may comprise more than one peptide in a single amino acid chain, which may be separated by a spacer, a linker or peptide cleavage sequence. A polypeptide may be a fused polypeptide. A polypeptide or a protein may comprise one or more domains. A domain may be a structural portion of a protein with a defined function, a polypeptide or a protein may comprise one or more modules. A module, in some cases, may be a domain or a portion of the domain or portion of a protein with a specific function. In some cases, a module may be a structural module of a protein, designated by its structural embodiments. In some cases, a moiety may be a portion of polypeptide, a protein or a nucleic acid, having a specific structure or perform a specific function. For example, a signaling moiety may be a specific unit within the larger structure of the polypeptide or protein or a recombinant nucleic acid, which (or the protein portion encoded by it in case of a nucleic acid) engages in a signal transduction process, for example a phosphorylation. In some cases, a module, a domain and a moiety may be terms that are used interchangeably, unless a specific structural or functional orientation is otherwise defined in the text. A motif may be a structural entity in a biomolecule. A signaling motif in a protein or polypeptide, for example, may refer to a stretch of amino acids on the protein or polypeptide which contain an amino acid which may be phosphorylated, dephosphorylated or may serve as a binding site of another signaling molecule. Similarly, in case of nucleic acids, for example, TNF mRNA has a conserved motif, UUAUUUAUU, in the 3′UTR to which mRNA destabilizing enzymes such as zinc-finger binding protein 36 family members bind.

As used herein, in some cases a “fragment” may refer to a molecule consisting of only a part of the intact full-length sequence and structure. The fragment may include a C-terminal deletion an N-terminal deletion, and/or an internal deletion of the polypeptide. Active fragments of a particular protein or polypeptide may include at least about 5-10 contiguous amino acid residues of the full length molecule, preferably at least about 15-25 contiguous amino acid residues of the full length molecule, and most preferably at least about 20-50 or more contiguous amino acid residues of the full length molecule, or any integer between 5 amino acids and the full length sequence, provided that the fragment in question retains biological activity, such as catalytic activity, ligand binding activity, regulatory activity, degron protein degradation signaling, or fluorescence characteristics.

In some cases, the disclosure comprises methods and use of a degron. In some embodiments, a “degron” may be an amino acid sequence that targets a protein for cellular degradation and specifies degradation of itself and any fusion protein of which it is a part. The degron may promote degradation of an attached polypeptide, for example, through either the proteasome or autophagy-lysosome pathways.

Some cases describe receptor dimerization or oligomerization or multimerization, which may refer to a property of some transmembrane proteins, e.g., some receptors to bind to each other and form a cluster usually with a functional consequence. In some embodiments, receptor dimerization may activate the receptors. In some embodiments, receptor dimerization may enhance receptor activity. In some cases, certain receptors may be activated by dimerization, that is, two molecules come in close proximity, are structurally linked or bound or are held by a third entity that both unit molecules individually bind to. In some embodiments, the dimerization may occur between two molecules of the same type, which may be referred to as homodimerization. In cases where the two molecules are not the same kind, such a dimerization may be referred to as heterodimerization. In some cases, more than two or but less than, for example ten or for example 3, 4, 5, 6, 7 etc. molecules cluster to form a complex held together structurally or by a function, such clustering may be referred to as oligomerization, or multimerization. In some cases, a multimerization may refer to less than arbitrarily about 10, 10, or more than 10 individual units or receptor molecules in a cluster.

In some embodiments, the term “recombinant nucleic acid molecule” may refer to a recombinant DNA molecule or a recombinant RNA molecule. A recombinant nucleic acid molecule may be any nucleic acid molecule containing joined nucleic acid molecules from different original sources and not naturally attached together. A recombinant nucleic acid may be synthesized in the laboratory. A recombinant nucleic acid may be prepared by using recombinant DNA technology by using enzymatic modification of DNA, such as enzymatic restriction digestion, ligation, and DNA cloning. A recombinant nucleic acid as used herein may be DNA, or RNA. A recombinant DNA may be transcribed in vitro, to generate a messenger RNA (mRNA), the recombinant mRNA may be isolated, purified and used to transfect a cell. A recombinant nucleic acid may encode a protein or a polypeptide. A recombinant nucleic acid, under suitable conditions, may be incorporated into a living cell, and can be expressed inside the living cell. As used herein, “expression” of a nucleic acid usually refers to transcription and/or translation of the nucleic acid. The product of a nucleic acid expression is usually a protein but may also be an mRNA. Detection of an mRNA encoded by a recombinant nucleic acid in a cell that has incorporated the recombinant nucleic acid, may be considered positive proof that the nucleic acid is “expressed” in the cell.

The process of inserting or incorporating a nucleic acid into a cell may be via transformation, transfection or transduction. In some embodiments, transformation may refer to the process of uptake of foreign nucleic acid by a bacterial cell. This process may be adapted for propagation of plasmid DNA, protein production, and other applications. Transformation may refer to introduction of recombinant plasmid DNA into competent bacterial cells that take up extracellular DNA from the environment. Some bacterial species may be naturally competent under certain environmental conditions, but competence may be artificially induced in a laboratory setting. Transfection may refer to the forced introduction of small molecules such as DNA, RNA, or antibodies into eukaryotic cells. Transfection may also refer to the introduction of bacteriophage into bacterial cells. “Transduction” may be used to describe the introduction of recombinant viral vector particles into target cells, while “infection” may refer to natural infections of humans or animals with wild-type viruses.

As used herein, in some embodiments, the term “vector” may refer to any genetic construct, such as a plasmid, phage, transposon, cosmid, chromosome, virus, virion, etc., which is capable transferring nucleic acids between cells. Vectors may be capable of one or more of replication, expression, recombination, insertion or integration, but need not possess each of these capabilities. In some embodiments, the term “vector.” A vector may refer to a nucleic acid sequence containing an origin of replication and other entities necessary for replication and/or maintenance in a host cell. Vectors may comprise a nucleic acid sequence that has a coding sequence and that is operably (e.g., operatively) linked with one or more regulatory elements that may utilize a cellular transcription machinery to transcribe the coding sequence, followed by translation when inside a living cell, thereby expressing the coding sequence. In some cases, expression may refer to production of an mRNA from a DNA sequence. In some cases, expression may refer to production of a peptide or polypeptide encoded by the nucleic acid. Vectors capable of directing the expression of genes and/or nucleic acid sequence to which they are operatively linked may be referred to herein as “expression vectors”. Expression vectors of utility may be in the form of “plasmids” which may refer to circular double stranded DNA molecules which, in their vector form are not bound to the chromosome, and may comprise entities for stable or transient expression or the encoded DNA. Other expression vectors that may be used in the methods as disclosed herein may include, but is not limited to, plasmids, episomes, bacterial artificial chromosomes, yeast artificial chromosomes, bacteriophages or viral vectors, and such vectors may integrate into the host's genome or replicate autonomously in the cell. A vector may be a DNA or RNA vector. Other forms of expression vectors known by those skilled in the art which serve the equivalent functions may also be used, for example, self-replicating extrachromosomal vectors or vectors capable of integrating into a host genome. Exemplary vectors may include those capable of autonomous replication and/or expression of nucleic acids to which they are linked.

In some embodiments, use of “regulatory” function or action may represent a meaning of both up-regulation and down-regulation, e.g., of a gene product. In some embodiments, up-regulation may signify induction, e.g., of a gene product, e.g., transcriptional upregulation. In some embodiments, regulation may represent control or checking action, e.g., suppression of an action. One of skill is able to use the context for more specific guidance.

The terms “spacer” or “linker” as used in reference to a fusion protein may refer to a peptide that joins the proteins comprising a fusion protein. In some embodiments, the constituent amino acids of a spacer may be selected to influence some property of the molecule such as the folding, net charge, or hydrophobicity of the molecule. Suitable linkers for use in an embodiment of the present disclosure are well known to those of skill in the art and may include, but is not limited to, straight or branched-chain carbon linkers, heterocyclic carbon linkers, or peptide linkers. The linker may be used to separate two antigenic peptides by a distance sufficient to ensure that, in some embodiments, each antigenic peptide properly folds. Exemplary peptide linker sequences may adopt a flexible extended conformation and do not exhibit a propensity for developing an ordered secondary structure. Typical amino acids in flexible protein regions may include Gly, Asn and Ser. Virtually any permutation of amino acid sequences containing Gly, Asn and Ser would be expected to satisfy the above criteria for a linker sequence. Other near neutral amino acids, such as Thr and Ala, may also be used in the linker sequence.

In some embodiments, the peptide linkers may have more than one functional property, such as the ones described herein. For example, the peptide linker may link two or more functional domains, such as binding domains. Additionally, the peptide linker may be a specific signal inducer when the linker contacts an extracellular portion of a cell, such as a receptor or a ligand binding protein.

The terms “neoplasia” and “cancer” may refer to any disease that is caused by or results in inappropriately high levels of cell division, inappropriately low levels of apoptosis, or both. Glioblastoma is one non-limiting example of a neoplasia or cancer. The terms “cancer” or “tumor” or “hyperproliferative disorder” may refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within an animal, or may be a non-tumorigenic cancer cell, such as a leukemia cell.

In some embodiments, the term “vaccine” may refer to a composition for generating immunity for the prophylaxis and/or treatment of diseases (e.g., neoplasia/tumor/infectious agents/autoimmune diseases). Accordingly, vaccines as used herein may be medicaments which comprise recombinant nucleic acids, or cells comprising and expressing a recombinant nucleic acid and are intended to be used in humans or animals for generating specific defense and protective substance by vaccination. A “vaccine composition” may include a pharmaceutically acceptable excipient, carrier or diluent. Aspects of the present disclosure relate to use of the technology in preparing a phagocytic cell-based vaccine.

In some embodiments, the term “pharmaceutically acceptable” may refer to approved or approvable by a regulatory agency of the Federal or a state government or listed in the U.S. Pharmacopeia or other generally recognized pharmacopeia for use in animals, including humans. A “pharmaceutically acceptable excipient, carrier or diluent” may refer to an excipient, carrier or diluent that can be administered to a subject, together with an agent, and which does not destroy the pharmacological activity thereof and is nontoxic when administered in doses sufficient to deliver a therapeutic amount of the agent. A “pharmaceutically acceptable salt” of pooled disease specific antigens as recited herein may be an acid or base salt that is generally considered in the art to be suitable for use in contact with the tissues of human beings or animals without excessive toxicity, irritation, allergic response, or other problem or complication. Such salts may include mineral and organic acid salts of basic residues such as amines, as well as alkali or organic salts of acidic residues such as carboxylic acids. Specific pharmaceutical salts may include, but is not limited to, salts of acids such as hydrochloric, phosphoric, hydrobromic, malic, glycolic, fumaric, sulfuric, sulfamic, sulfanilic, formic, toluene sulfonic, methane sulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH2)n-COOH where n is 0-4, and the like. Similarly, pharmaceutically acceptable cations may include, but is not limited to sodium, potassium, calcium, aluminum, lithium and ammonium. Those of ordinary skill in the art will recognize from this disclosure and the knowledge in the art that further pharmaceutically acceptable salts for the pooled disease specific antigens provided herein, including those listed by Remington's Pharmaceutical Sciences, 17th ed., Mack Publishing Company, Easton, PA, p. 1418 (1985).

Nucleic acid molecules useful in the methods of the disclosure may include any nucleic acid molecule that encodes a polypeptide of the disclosure or a fragment thereof. Such nucleic acid molecules need not be 100% identical with an endogenous nucleic acid sequence, but will typically exhibit substantial identity. Polynucleotides having substantial identity to an endogenous sequence may be capable of hybridizing with at least one strand of a double-stranded nucleic acid molecule. “Hybridize” may refer to when nucleic acid molecules pair to form a double-stranded molecule between complementary polynucleotide sequences, or portions thereof, under various conditions of stringency. For example, stringent salt concentration may be less than about 750 mM NaCl and 75 mM trisodium citrate, less than about 500 mM NaCl and 50 mM trisodium citrate, or less than about 250 mM NaCl and 25 mM trisodium citrate. Low stringency hybridization may be obtained in the absence of organic solvent, e.g., formamide, while high stringency hybridization may be obtained in the presence of at least about 35% formamide, or at least about 50% formamide. Stringent temperature conditions may include temperatures of at least about 30° C., at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency may be accomplished by combining these various conditions as needed. In an exemplary embodiment, hybridization may occur at 30° C. in 750 mM NaCl, 75 mM trisodium citrate, and 1% SDS. In another exemplary embodiment, hybridization may occur at 37° C. in 500 mM NaCl, 50 mM trisodium citrate, 1% SDS, 35% formamide, and 100 mg/ml denatured salmon sperm DNA (ssDNA). In another exemplary embodiment, hybridization may occur at 42° C. in 250 mM NaCl, 25 mM trisodium citrate, 1% SDS, 50% formamide, and 200 mg/ml ssDNA. Useful variations on these conditions will be readily apparent to those skilled in the art. For most applications, washing steps that follow hybridization may also vary in stringency. Wash stringency conditions may be defined by salt concentration and by temperature. As above, wash stringency may be increased by decreasing salt concentration or by increasing temperature. For example, stringent salt concentration for the wash steps may be less than about 30 mM NaCl and 3 mM trisodium citrate, or less than about 15 mM NaCl and 1.5 mM trisodium citrate. Stringent temperature conditions for the wash steps may include a temperature of at least about 25° C., of at least about 42° C., or at least about 68° C. In exemplary embodiments, wash steps may occur at 25° C. in 30 mM NaCl, 3 mM trisodium citrate, and 0.1% SDS. In other exemplary embodiments, wash steps may occur at 42° C. in 15 mM NaCl, 1.5 mM trisodium citrate, and 0.1% SDS. In another exemplary embodiment, wash steps may occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

“Substantially identical” may refer to a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Such a sequence may be at least 60%, 80% or 85%, 90%, 95%, 96%, 97%, 98%, or even 99% or more identical at the amino acid level or nucleic acid to the sequence used for comparison. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions may include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-m^{o} indicating a closely related sequence. A “reference” may refer to a standard of comparison.

In some embodiments, the term “subject” or “patient” may refer to an animal which is the object of treatment, observation, or experiment. By way of example only, a subject may include, but is not limited to, a mammal, including, but not limited to, a human or a non-human mammal, such as a non-human primate, murine, bovine, equine, canine, ovine, or feline.

The terms “treat,” “treated,” “treating,” “treatment,” and the like may refer to reducing, preventing, or ameliorating a disorder and/or symptoms associated therewith (e.g., a neoplasia or tumor or infectious agent or an autoimmune disease). “Treating” may refer to administration of the therapy to a subject after the onset, or suspected onset, of a disease (e.g., cancer or infection by an infectious agent or an autoimmune disease). “Treating” may include the concepts of “alleviating”, which may refer to lessening the frequency of occurrence or recurrence, or the severity, of any symptoms or other ill effects related to the disease and/or the side effects associated with therapy. In some embodiments, the term “treating” may also encompass the concept of “managing” which may refer to reducing the severity of a disease or disorder in a patient, e.g., extending the life or prolonging the survivability of a patient with the disease, or delaying its recurrence, e.g., lengthening the period of remission in a patient who had suffered from the disease. It is appreciated that, although not precluded, treating a disorder or condition may not require that the disorder, condition, or symptoms associated therewith be completely eliminated.

In some embodiments, the term “prevent”, “preventing”, “prevention” and their grammatical equivalents as used herein, may refer to avoiding or delaying the onset of symptoms associated with a disease or condition in a subject that has not developed such symptoms at the time the administering of an agent or compound commences.

In some embodiments, the term “therapeutic effect” may refer to some extent of relief of one or more of the symptoms of a disorder (e.g., a neoplasia, tumor, or infection by an infectious agent or an autoimmune disease) or its associated pathology. “Therapeutically effective amount” as used herein may refer to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. “Therapeutically effective amount” may qualify the amount required to achieve a therapeutic effect. A physician or veterinarian having ordinary skill in the art can readily determine and prescribe the “therapeutically effective amount” (e.g., ED50) of the pharmaceutical composition required.

As used herein, in some embodiments, the term “affinity molecule” may refer to a molecule or a ligand that binds with chemical specificity to an affinity acceptor peptide. Chemical specificity may refer to the ability of a protein's binding site to bind specific ligands. The fewer ligands a protein can bind, the greater its specificity. Specificity may describe the strength of binding between a given protein and ligand. This relationship may be described by a first scFv specific to a cell surface component on a dissociation constant (KD), which may characterize the balance between bound and unbound states for the protein-ligand system.

Reference in the specification to “some embodiments,” “an embodiment,” “one embodiment” or “other embodiments” may mean that a feature, structure, or characteristic described in connection with the embodiments is included in at least some embodiments, but not necessarily all embodiments, of the present disclosure.

In some embodiments, the term “myeloid cells” may be used herein to mean the cell lineage originating from the bone marrow that includes polymorphonuclear neutrophils, eosinophils, basophils, and mast cells, as well as the monocyte/macrophage lineage and different dendritic cell lineages. Myeloid cells are not capable of differentiating into lymphoid cells (e.g., NK-, B- and T-lymphocytes). In some embodiments, the term may refer to cells of the myeloid lineages in all stages of their differentiation and therefore may include hematopoietic blast cells, i.e., hematopoietic cells that are committed to the myeloid cell lineage, but that are in early stages of differentiation. When stimulated with appropriate growth factors, hematopoietic blast cells divide to produce a large number of cells that are more differentiated than the blast stage of differentiation. Examples may include, inter alia, myeloblasts. Although macrophages are exemplified throughout the specification, the compositions and methods described here may be applicable to cells of a myeloid cell lineage, such as a dendritic cell. Minor optimizations and changes are envisioned on a cell-to-cell basis as is known to one of skill in the art, and is contemplated within the scope of the invention.

Cells that are more differentiated than blasts but not yet fully differentiated are appended with the prefix “pro” and are also intended to fall under the definition of “myeloid cells.” Examples are promyelocytes.

In some embodiments, the term “myeloid cells” may also include myeloid progenitor cells, i.e., cell lineages, e.g., in the bone marrow, that are capable of differentiating in cells such as myelomonocytic progenitor cells, proerythroblasts or immature megakaryoblasts. Myeloid progenitor cells are not capable of giving rise to lymphoid cells.

In some embodiments, the term “myeloid cells” may not include lympho-hematopoietic stem cells. Lympho-hematopoietic stem cells are defined as those cells that are capable of both self-renewal and differentiation into the two principle precursor components, the myeloid and lymphoid lines. Such stem cells are said to be totipotent. Stem cells that are less general but that can still differentiate into several lines are called pluripotent.

The present disclosure involves programming myeloid cells in vivo or ex vivo, making and using engineered myeloid cells (e.g., CD14+ cells, such as macrophages or other phagocytic cells, which can attack and kill (ATAK) diseased cells directly and/or indirectly, such as cancer cells and infected cells. Engineered myeloid cells, such as macrophages and other phagocytic cells, can be prepared by incorporating nucleic acid sequences (e.g., mRNA, plasmids, viral constructs) encoding a chimeric fusion protein (CFP), that has an extracellular binding domain specific to disease associated antigens (e.g., cancer antigens), into the cells using, for example, ex vivo using recombinant nucleic acid technology, synthetic nucleic acids, gene editing techniques (e.g., CRISPR), transduction (e.g., using viral constructs), electroporation, or nucleofection, or in vivo using mRNA delivery technology including but not limited to LNP technology. It has been found that myeloid cells can be engineered to have a broad and diverse range of activities. For example, it has been found that myeloid cells can be engineered to express a chimeric fusion protein (CFP) containing an antigen binding domain to have a broad and diverse range of activities. For example, it has been found that myeloid cells can be engineered to have enhanced phagocytic activity such that upon binding of the CFP to an antigen on a target cell, the cell exhibits increased phagocytosis of the target cell. It has also been found that myeloid cells can be engineered to promote T cell activation such that upon binding of the CFP to an antigen on a target cell, the cell promotes activation of T cells, such as T cells in the tumor microenvironment. Myeloid cells can be engineered to promote secretion of tumoricidal molecules such that upon binding of the CFP to an antigen on a target cell, the cell promotes secretion of tumoricidal molecules from nearby cells. Myeloid cells can be engineered to promote recruitment and trafficking of immune cells and molecules such that upon binding of the CFP to an antigen on a target cell, the cell promotes recruitment and trafficking of immune cells and molecules to the target cell or a tumor microenvironment.

One of the primary hurdles of using immune cells in therapy lies in generating precise action by particular immune cells at a particular time. For example, in a natural healthy system, immune cells are activated temporally, and they play a cytotoxic role precisely on a microbe or a diseased cell, and the inflammatory and cytotoxic responses are rapidly controlled by the system to return to baseline levels. In cases of prolonged and persistent disease conditions, both the tunability and the return to baseline are compromised, and immune cells can become either naturally unresponsive and anergic or persistently inflammatory which damages the tissue or the system further. Similarly, artificially inducing the immune systems to act on a pathogen or a pathogenic cell, e.g., a cancer cell, an approach adopted by immune cell therapy can be highly challenging to generate a precise control of induction of cytotoxic function, where persistent stimuli may already exist in the diseased system. On the other hand, it is necessary to prevent persistent inflammation caused by overexpression of an activator inside a cell, for example, by halting the overexpression. Tailoring a response precisely at the time of contact between an immune cell with a pathogen or a pathogenic cell is of essence in such therapeutic approaches.

An aspect of the instant disclosure is the generation of tunable CAR expression specifically in myeloid cells that (i) target a specific cancer cell; (ii) act upon binding to the target cancer cell by activating an inducible transcriptional activator, and (iii) halt the response once the transcriptional activator is activated. In some embodiments, the process does not allow tonic activation of the system, and does not allow persistent rounds of activation once the activation has been set in motion. The system disclosed herein therefore generates transient, robust and effect immune cells targeted to the pathogenic cell, e.g., a cancer cell.

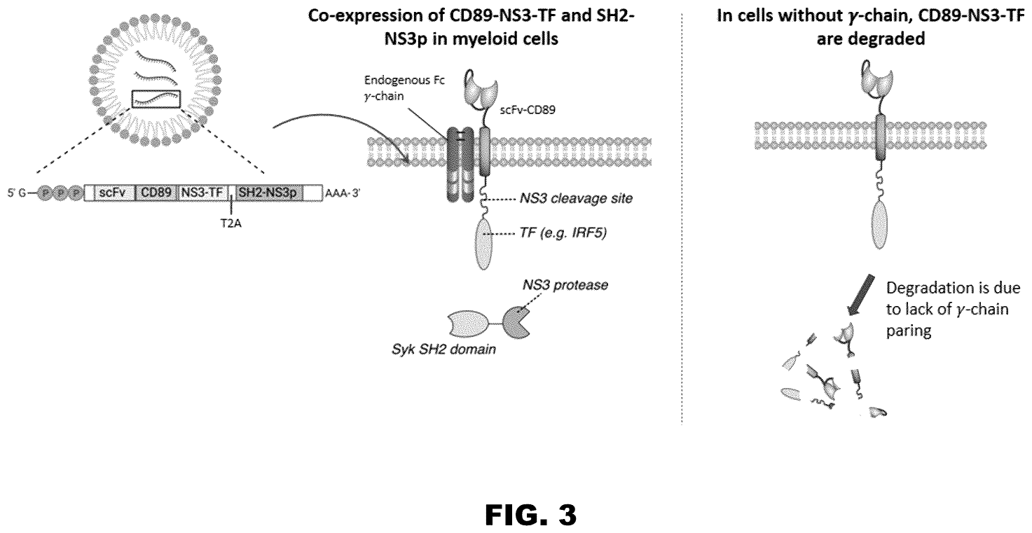

In one aspect of the disclosure, provided herein are cell surface receptors that express in myeloid cells, or express predominantly in myeloid cells. In some embodiments, provided herein is a recombinant polynucleic acid, such that the recombinant polynucleic acid, packaged in a composition comprising a suitable delivery vehicle, can be introduced systemically in a subject in need of therapy, and the recombinant nucleic acid is expressed predominantly in myeloid cells. This may be achieved by the design of the chimeric fusion protein as disclosed herein, which leverages the fact that certain cells predominantly express certain proteins endogenously, and the expression and activation of some other proteins depend on the presence and action of the endogenously expressed protein. For example, in this context, some proteins present co-stimulatory domains, other proteins have domains that structurally form a complex that stabilizes the proteins and/or render them functional in a cell type. In some embodiments, this is designed to achieve a myeloid cell specific expression by introducing a chimeric receptor with a transmembrane domain that oligomerizes with endogenous Fc receptor transmembrane domains predominantly expressed on myeloid cells and thereby stabilizes the chimeric receptor. As illustrated in FIG. 3 (right), the chimeric fusion protein degrades readily in cells that do not express endogenous Fcγ-receptors. For example, Fcγ-receptors are not expressed in lymphoid cells, e.g., T cells. Therefore, having a transmembrane domain that dimerizes or oligomerizes with endogenous Fcγ-receptor transmembrane domains provides an advantage towards selectively expressing the construct predominantly in myeloid cells, and avoiding the lymphoid cells. Therefore, accordingly, in some embodiments, the transmembrane domain is a transmembrane domain from a protein that dimerizes with Fcγ-receptor. Proteins with transmembrane domains that dimerize or oligomerize with Fc receptor transmembrane domains in a myeloid cell include but are not limited to CD16a, CD64, CD68 and CD89.

In some embodiments, the transmembrane domain is a transmembrane domain from a protein that dimerizes with endogenous FcR-gamma receptors in myeloid cells, monocytes or macrophages; wherein after administration of the composition to a human subject the recombinant polynucleic acid encoding the chimeric fusion protein (CFP) is specifically expressed in myeloid cells, e.g., monocytes or macrophages of the human subject. In some embodiments, the transmembrane domain is a transmembrane domain from CD16a, CD64, CD68 or CD89. In some embodiments, the CFP further comprises an intracellular domain, wherein the intracellular domain comprises one or more intracellular signaling domains, and wherein the one or more intracellular signaling domains comprises an intracellular signaling domain from FcγR, FcαR, FcεR, CD40 or CD3zeta. In some embodiments, the one or more intracellular signaling domains further comprises a phosphoinositide 3-kinase (PI3K) recruitment domain. In some embodiments, the intracellular signaling domain attached to the aforementioned CFP with transmembrane domain from CD16a, CD64, CD68 or CD89 comprise one or more signaling domains, e.g., ITAM motif containing domains. In some embodiments, the intracellular signaling domains may be phosphorylated upon receptor engagement with target antigens. In some embodiments, the intracellular domain comprises an intracellular domain from CD16a, CD64, CD68 or CD89.

In some embodiments, an intracellular domain at the C terminus or intracellular side of the transmembrane domain from CD16a, CD64, CD68 or CD89 may not contain a signaling domain that has a PI3 kinase recruitment domain, by design. In some embodiments, an intracellular domain at the C terminus or intracellular end of a transmembrane domain derived from CD16a, CD64, CD68 or CD89 in a CFP may not contain a signaling domain that has a ITAM signaling domain, by design. In some embodiments, the FcγR intracellular domain is activated upon receptor engagement with a target antigen. Typically, FcγR intracellular domain receptor comprises tyrosine residues that are phosphorylated upon receptor activation. Phosphorylation of tyrosine residues upon receptor activation initiates a cascade of intracellular signaling events, wherein SH2-binding proteins are attracted to the receptor and are in turn phosphorylated and activated. In some embodiments, the intracellular domain of the chimeric receptor construct does not have an intracellular signaling domain or an intracellular domain comprising phosphotyrosine residues.

In some embodiments, the intracellular domain of the CFP comprises a sequence for a therapeutic agent. In some embodiments, the therapeutic agent is a transcription factor. In some embodiments, the therapeutic agent is a transcription activator. In some embodiments the therapeutic agent is a transcription repressor.

In some embodiments, the CFP is designed such that engagement of the extracellular antigen binding domain of the CFP with its ligand or antigen, activates the release of the therapeutic agent from its receptor bound form into an active form. In some embodiments, engagement of the ligand or antigen binding domain of the CFP with activates the release of the transcription factor for migrating inside the nucleus and binding to the cognate DNA for initiating transcription, or repressing transcription depending on the transcription factor. In general, a transcription factor is a transcription activator. However, as contemplated herein, a transcription factor can be a transcription repressor, e.g., a repressor of anti-inflammatory genes that suppress monocyte/macrophage activation and phagocytosis. Accordingly, in one design of the chimeric receptor, the sequence encoding the therapeutic agent, e.g., the transcription factor is separated from the transmembrane domain or the associated intracellular domain of the CFP by a regulatable domain, that can be cleaved to release the therapeutic agent. The regulatable domain is a domain that can be cleaved by an intracellular protease. Other regulatable domains that can be conceived for achieving the goal sought herein may include but not limited to a dimerization domain, zinc finger domains, a leucine zipper domain, a helix-loop-helix domain, trypsin sensitive domain, and sulfide bridge connections, and the technology may be likewise modified or designed to suit the regulatable domain.

In some embodiments, the recombinant nucleic acid or polynucleic acid comprises the sequence encoding the chimeric protein as described above, and a sequence encoding a regulator that can act on the regulatable domain such that when the regulator acts on the regulatable domain, the therapeutic agent is released. The sequence encoding the regulator may be preferably separated from the sequence encoding the CFP by a post-translationally modifiable, or auto-cleavable domain or region. Examples of post-translationally auto-cleavable domains can be T2A, or P2A. The regulator domain can be produced co-translationally with the CFP, and then post-translationally cleaved. In some embodiments, a regulatable domain can be a protease sensitive domain and the regulator can be a protease. At certain predetermined times, contacting by the regulator to the regulatable domain results in detachment of the therapeutic agent, e.g., the transcription factor from the remainder of the CFP. In some other conceivable and exemplary designs, a regulatable domain is a domain that is protected upon remaining bound to the regulator, and detachment of the regulator may cause release of the therapeutic agent from the remainder of the CFP.

Accordingly, in some embodiments, provided herein is a recombinant polynucleic acid, wherein the recombinant polynucleic acid comprises a sequence encoding a cell surface receptor, wherein the cell surface receptor is a chimeric fusion protein (CFP) comprising: (a) an extracellular domain comprising an antigen binding domain, (b) a transmembrane domain operatively linked to the extracellular domain; and (c) an intracellular domain operatively linked to the transmembrane domain, the intracellular domain comprising: a therapeutic agent, wherein the therapeutic agent is a transcription factor or transcriptionally functional portion thereof, and a protease cleavage site disposed between the transcription factor and the transmembrane domain.

In one aspect, provided herein is a composition comprising a recombinant polynucleic acid, wherein the recombinant polynucleic acid comprises a sequence encoding a cell surface receptor, wherein the cell surface receptor is a chimeric fusion protein (CFP) comprising: (a) an extracellular domain comprising an antigen binding domain, (b) a transmembrane domain operatively linked to the extracellular domain, wherein the transmembrane domain is a transmembrane domain from a protein that dimerizes with Fc-gamma receptor; and (c) an intracellular domain operatively linked to the transmembrane domain, the intracellular domain comprising: a therapeutic agent, and a protease cleavage site disposed between the therapeutic agent and the transmembrane domain. wherein cleavage of the protease cleavage site by the protease releases the therapeutic agent from the CFP.

In some embodiments, the therapeutic agent is a transcription factor or transcriptionally functional portion thereof. In some embodiments, the transcription factor is an inflammatory response transcription factor. In some embodiments, the transcription factor activates an interferon response gene, such as IRF3, or IRF5.

In some embodiments, the regulator is a fusion protein. In some embodiments, the regulator comprises an SH2-binding domain that is fused to the protease. In some embodiments, the protease is maintained in inactive or suppressed state by a protease inhibitor. In this way, a regulator may be used to regulate the intracellular activation of the therapeutic agent, that is the transcription factor, and may in turn be regulated from outside by administration of a protease inhibitor that maintains it in an inactive state for a period of time.

In some embodiments, the first fusion protein encoded by the recombinant polynucleic acid is the chimeric fusion protein comprising a transcription factor for inducing proinflammatory gene transcription upon chimeric receptor engagement with its target ligand or antigen on a cancer cell.

In some embodiments, the second fusion protein encoded by the recombinant polynucleic acid is the fusion protein comprising a protease or a functional fragment thereof, which is fused to a signal recognition domain such as a phosphotyrosine binding domain, e.g., an SH2 domain or an SHC domain; and optionally linked to a protease inhibitor, and further optionally linked to a degron.

In some embodiments, the first fusion protein and the second fusion protein are encoded by the same recombinant polynucleic acid that are separated by a T2A or P2A self-cleavable sequence and are co-translationally or post-translationally cleaved and separated.

In some embodiments, the first fusion protein and the second fusion protein are encoded by separate mRNA that are packaged in a single nanoparticle. In some embodiments, the first fusion protein and the second fusion protein are encoded by separate mRNA that are not packaged in a single nanoparticle.

### First Fusion Protein: Chimeric Fusion Protein and Transcription Factor

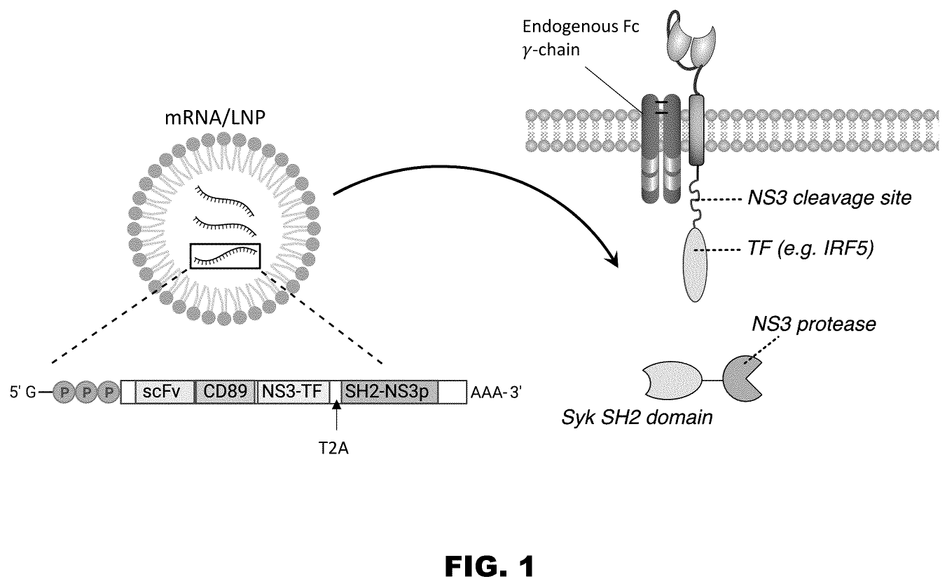

In one aspect, provided herein is a composition comprising a recombinant polynucleic acid, wherein the recombinant polynucleic acid comprises a sequence encoding a cell surface receptor, wherein the cell surface receptor is a chimeric fusion protein (CFP) comprising: (a) an extracellular domain comprising an antigen binding domain, (b) a transmembrane domain operatively linked to the extracellular domain; and (c) an intracellular domain operatively linked to the transmembrane domain, the intracellular domain comprising: (i) a therapeutic agent, wherein the therapeutic agent is a transcription factor or transcriptionally functional portion thereof, and (ii) a protease cleavage site disposed between the transcription factor and the transmembrane domain. The therapeutic agent may be in a deactivated, repressed or bound form until the extracellular antigen binding domain is in contact with the target antigen, which activates the CFP, and upon activation is amenable to a biochemical change, such that the therapeutic agent may be activated or released intracellularly and is available to perform a transcriptional activity. In some embodiments, the activation of the CFP causes intracellular activation of a third molecule, e.g., an activator which then activates or helps release the therapeutic agent that is a transcription factor or transcriptionally functional portion thereof. In some embodiments, the CFP is engineered such that by design, the CFP may be expressed only in a certain cell type and may not be expressed, may be unstable or may undergo degradation in other cell types. In some embodiments, the CFP may be engineered to comprise a transmembrane domain such that the CFP may express in the cell type where the transmembrane domain dimerizes or oligomerizes with one or more endogenous proteins and is stabilized, in absence of such endogenous proteins, the CFP may not be stabilized and is degraded or not expressed on the membrane, thereby offering cell-specificity in the engineered design of the CFP. For example, a CFP described herein, can include a FcR dimerization transmembrane domain, the CFP would successfully express on a myeloid cell, e.g., a monocyte, a macrophage, a dendritic cell, etc., but may not be expressed on a T cell. However, in another example, a fusion protein can include a TCR dimerization transmembrane domain, the fusion protein would successfully express on the membrane of a lymphoid cell, e.g. a T cell, and not be expressed on a myeloid cell. In some embodiments, the CFP may further comprise a recruitment domain for a third molecule, such as a protease, or other proteins or enzymes that may be bound only to an activated CFP at the intracellular region, and promote activation of the therapeutic agent that is a transcription factor or transcriptionally functional portion thereof. In some embodiments, the therapeutic agent may be an interferon responsive factor, e.g., IRF3 or IRF5; wherein therapeutic agent is separated from the transmembrane domain (or a proximal region of a cytoplasmic region of the CFP) via a NS3 cleavage sequence (FIGS. 1-4B). In some embodiments, the composition comprises a lipid nanoparticle (LNP), wherein the LNP may encapsulate the recombinant polynucleic acid comprises a sequence encoding the CFP.

In some embodiments, extracellular domain comprising an antigen binding domain of the CFP comprises the binding domain of an antibody, a functional fragment of an antibody, a variable domain thereof, a V_{H} domain, a V_{L} domain, a VNAR domain, a V_{HH} domain, a single chain variable fragment (scFv), an Fab, a single-domain antibody (sdAb), a nanobody, a bispecific antibody, a diabody, or a functional fragment or a combination thereof that is specific for binding to an antigen or a ligand on a target cell, e.g. a cancer cell. In some embodiments, the antigen on the target cell to which the first binding domain binds is a cancer antigen or a pathogenic antigen on the target cell or an autoimmune antigen. In some embodiments, the extracellular binding domain “specifically binds” to the antigen if it binds to or associates with the antigen with an affinity or Ka (that is, an equilibrium association constant of a particular binding interaction with units of 1/M) of, for example, greater than or equal to about 10\hat{ }6 M\hat{ }−1. In some embodiments, the extracellular binding domain binds to an antigen with a Ka greater than or equal to about 10\hat{ }6 M\hat{ }−1, 10\hat{ }7 M\hat{ }−1, 10\hat{ }8 M\hat{ }−1, 10\hat{ }9 M\hat{ }−1, 10\hat{ }10 M\hat{ }−1, 10\hat{ }11 M\hat{ }−1, 10\hat{ }12 M\hat{ }−1, or 10\hat{ }13 M\hat{ }−1. “High affinity” binding refers to binding with a Ka of at least 10\hat{ }7 M\hat{ }−1, at least 10\hat{ }8M\hat{ }−1, at least 109 M−1, at least 1010 M−1, at least 1011 M−1, at least 1012 M−1, at least 10\hat{ }13 M\hat{ }−1, or greater. Alternatively, affinity may be defined as an equilibrium dissociation constant of a particular binding interaction with units of M (e.g., 10\hat{ }−6 M to 10\hat{ }−13 M, or less). In some embodiments, specific binding means the extracellular binding domain binds to the target molecule with a KD of less than or equal to about 10\hat{ }−6 M, less than or equal to about 10\hat{ }−6 M, less than or equal to about 10\hat{ }−7 M, less than or equal to about 10\hat{ }−8 M, or less than or equal to about 10\hat{ }−9 M, 10\hat{ }−10 M, 10\hat{ }−11 M, or 10\hat{ }−12 M or less. The binding affinity of the extracellular binding domain for the target antigen can be readily determined using conventional techniques, e.g., by competitive ELISA (enzyme-linked immunosorbent assay), equilibrium dialysis, by using surface plasmon resonance (SPR) technology (e.g., the BIAcore 2000 instrument, using general procedures outlined by the manufacturer); by radioimmunoassay; among others.

In some embodiments, the antigen or ligand on a cancer cell can be a ligand or an antigen that expresses or overexpresses on a cancer cell. In some embodiments the ligand or antigen aberrantly expresses the ligand or antigen. In some embodiments the antigen binding domain of the CFP helps selective binding of the CFP preferentially or selectively on a cancer cell, relative to a non-cancer cell; sparing the corresponding non-cancer cells of the same tissue or origin.

Provided herein are compositions comprising a recombinant nucleic acid encoding a chimeric fusion protein (CFP), such as a phagocytic receptor (PR) fusion protein (PFP), a scavenger receptor (SR) fusion protein (SFP), an integrin receptor (IR) fusion protein (IFP) or a caspase-recruiting receptor (caspase-CAR) fusion protein. A CFP encoded by the recombinant nucleic acid can comprise an extracellular domain (ECD) comprising an antigen binding domain that binds to an antigen of a target cell. The extracellular domain can be fused to a hinge domain or an extracellular domain derived from a receptor, such as CD2, CD8, CD28, CD68, a phagocytic receptor, a scavenger receptor or an integrin receptor.

Provided herein is a composition comprising a recombinant nucleic acid encoding a CFP comprising a phagocytic or tethering receptor (PR) subunit (e.g., a phagocytic receptor fusion protein (PFP)) comprising: a transmembrane domain, an intracellular domain comprising a phagocytic receptor intracellular signaling domain; and an extracellular antigen binding domain specific to an antigen, e.g., an antigen of or presented on a target cell; wherein the transmembrane domain and the extracellular antigen binding domain are operatively linked such that antigen binding to the target by the extracellular antigen binding domain of the fused receptor activated in the intracellular signaling domain of the phagocytic receptor.

In some embodiments, the extracellular domain of a CFP comprises an Ig binding domain. In some embodiments, the extracellular domain comprises an IgA, IgD, IgE, IgG, IgM, FcRγI, FcRγIIA, FcRγIIB, FcRγIIC, FcRγIIIA, FcRγIIIB, FcRn, TRIM21, FcRL5 binding domain. In some embodiments, the extracellular domain of a CFP comprises an FcR extracellular domain. In some embodiments, the extracellular domain of a CFP comprises an FcRα, FcRβ, FGRε or FcRγ extracellular domain. In some embodiments, the extracellular domain comprises an FcRα (FCAR) extracellular domain. In some embodiments, the extracellular domain comprises an FcRβ extracellular domain. In some embodiments, the extracellular domain comprises an FCER1A extracellular domain. In some embodiments, the extracellular domain comprises an FDGR1A, FCGR2A, FCGR2B, FCGR2C, FCGR3A, or FCGR3B extracellular domain. In some embodiments, the extracellular domain comprises an integrin domain or an integrin receptor domain. In some embodiments, the extracellular domain comprises one or more integrin α1, α2, αIIb, α3, α4, α5, α6, α7, α8, α9, α10, α11, αD, αE, αL, αM, αV, αX, β1, β2, β3, β4, β5, β6, β7, or β8 domains.

In some embodiments, the CFP further comprises an extracellular domain operatively linked to the transmembrane domain and the extracellular antigen binding domain. In some embodiments, the extracellular domain further comprises an extracellular domain of a receptor, a hinge, a spacer and/or a linker. In some embodiments, the extracellular domain comprises an extracellular portion of a phagocytic receptor. In some embodiments, the extracellular portion of the CFP is derived from the same receptor as the receptor from which the intracellular signaling domain is derived. In some embodiments, the extracellular domain comprises an extracellular domain of a scavenger receptor. In some embodiments, the extracellular domain comprises an immunoglobulin domain. In some embodiments, the immunoglobulin domain comprises an extracellular domain of an immunoglobulin or an immunoglobulin hinge region. In some embodiments, the extracellular domain comprises a phagocytic engulfment domain. In some embodiments, the extracellular domain comprises a structure capable of multimeric assembly. In some embodiments, the extracellular domain comprises a scaffold for multimerization. In some embodiments, the extracellular domain is at least 10, 20, 30, 40, 50, 60, 70, 80, 90, 100, 150, 200, 300, 300, 400, or 500 amino acids in length. In some embodiments, the extracellular domain is at most 500, 400, 300, 200, or 100 amino acids in length. In some embodiments, the extracellular antigen binding domain specifically binds to the antigen of a target cell. In some embodiments, the extracellular antigen binding domain comprises an antibody domain. In some embodiments, the extracellular antigen binding domain comprises a receptor domain, antibody domain, wherein the antibody domain comprises a functional antibody fragment, a single chain variable fragment (scFv), an Fab, a single-domain antibody (sdAb), a nanobody, a V_{H} domain, a V_{L} domain, a VNAR domain, a V_{HH} domain, a bispecific antibody, a diabody, or a functional fragment or a combination thereof. In some embodiments, the extracellular antigen binding domain comprises a ligand, an extracellular domain of a receptor or an adaptor. In some embodiments, the extracellular antigen binding domain comprises a single extracellular antigen binding domain that is specific for a single antigen. In some embodiments, the extracellular antigen binding domain comprises at least two extracellular antigen binding domains, wherein each of the at least two extracellular antigen binding domains is specific for a different antigen.

In some embodiments, the antigen is a cancer associated antigen, a lineage associated antigen, a pathogenic antigen or an autoimmune antigen. In some embodiments, the antigen comprises a viral antigen. In some embodiments, the antigen is a T lymphocyte antigen. In some embodiments, the antigen is an extracellular antigen. In some embodiments, the antigen is an intracellular antigen. In some embodiments, the antigen is selected from the group consisting of an antigen from Thymidine Kinase (TK1), Hypoxanthine-Guanine Phosphoribosyltransferase (HPRT), Receptor Tyrosine Kinase-Like Orphan Receptor 1 (ROR1), Mucin-1, Mucin-16 (MUC16), MUC1, Epidermal Growth Factor Receptor vIII (EGFRvIII), Mesothelin, Human Epidermal Growth Factor Receptor 2 (HER2), EBNA-1, LEMD1, Phosphatidyl Serine, Carcinoembryonic Antigen (CEA), B-Cell Maturation Antigen (BCMA), Glypican 3 (GPC3), Follicular Stimulating Hormone receptor, Fibroblast Activation Protein (FAP), Erythropoietin-Producing Hepatocellular Carcinoma A2 (EphA2), EphB2, a Natural Killer Group 2D (NKG2D) ligand, Disialoganglioside 2 (GD2), CD2, CD3, CD4, CD5, CD7, CD8, CD19, CD20, CD22, CD24, CD30, CD33, CD38, CD44v6, CD45, CD56CD79b, CD97, CD117, CD123, CD133, CD138, CD171, CD179a, CD213A2, CD248, CD276, PSCA, CS-1, CLECL1, GD3, PSMA, FLT3, TAG72, EPCAM, IL-1, an integrin receptor, PRSS21, VEGFR2, PDGFRβ, SSEA-4, EGFR, NCAM, prostase, PAP, ELF2M, GM3, TEM7R, CLDN6, TSHR, GPRC5D, ALK, Dsg1, Dsg3, IGLL1 and combinations thereof. In some embodiments, the antigen is an antigen of a protein selected from the group consisting of CD2, CD3, CD4, CD5, CD7, CCR4, CD8, CD30, CD45, and CD56. In some embodiments, the antigen is an ovarian cancer antigen or a T lymphoma antigen. In some embodiments, the antigen is an antigen of an integrin receptor. In some embodiments, the antigen is an antigen of an integrin receptor or integrin selected from the group consisting of α1, α2, αIIb, α3, α4, α5, α6, α7, α8, α9, α10, α11, αD, αE, αL, αM, αV, αX, β1, β2, β3, β4, β5, β6, β7, and β8. In some embodiment, the antigen is an antigen of an integrin receptor ligand. In some embodiments, the antigen is an antigen of fibronectin, vitronectin, collagen, or laminin. In some embodiments, the antigen binding domain can bind to two or more different antigens. Additional tumor-associated molecule or tumor-specific molecule may be selected as target from HER2, B7-H3 (CD276), CD19, CD20, GD2, CD22, CD30, CD33, CD56, CD66/CEACAM5, CD70, CD74, CD79b, CD123, CD133 CD138, CD171, B-cell maturation antigen (BCMA), Nectin-4, Mesothelin, Transmembrane glycoprotein NMB (GPNMB), Prostate-Specific Membrane Antigen (PSMA), SLC44A4, CA6, tyrosine-protein kinase Met (c-Met), epidermal growth factor receptor variant III (EGFRvIII), mucin 1 (MUC1), ephrin type-A receptor 2 (EphA2), glypican 2 (GPC2), glypican 3 (GPC3), fms-like tyrosine kinase 3 (FLT3), folate receptor alpha (FRa), IL-13 receptor alpha 2 (IL13Ra2), fibroblast activation protein (FAP), receptor tyrosine kinase-like orphan receptor 1 (ROR1), delta-like 3 (DLL3), K light chain, vascular endothelial growth factor receptor 2 (VEGFR2), Trophoblast glycoprotein (TPBG), anaplastic lymphoma kinase (ALK), CA-IX, an integrin, C-X-C chemokine receptor type 4 (CXCR4), neuropilin-1 (NRP1), matriptase.

In some embodiments, the antigen binding domain comprises an autoantigen or fragment thereof, such as Dsg1 or Dsg3. In some embodiments, the extracellular antigen binding domain comprises a receptor domain or an antibody domain wherein the antibody domain binds to an auto antigen, such as Dsg1 or Dsg3.

In some embodiments, the transmembrane domain and the extracellular antigen binding domain are operatively linked through a linker. In some embodiments, the transmembrane domain and the extracellular antigen binding domain are operatively linked through a linker such as a hinge region of CD8a, IgG1 or IgG4.

In some embodiments, the extracellular domain comprises a multimerization scaffold.

In some embodiments, the transmembrane domain comprises a CD8 transmembrane domain. In some embodiments, the transmembrane domain comprises a CD28 transmembrane domain. In some embodiments, the transmembrane domain comprises a CD68 transmembrane domain. In some embodiments, the transmembrane domain comprises a CD2 transmembrane domain. In some embodiments, the transmembrane domain comprises an FcR transmembrane domain. In some embodiments, the transmembrane domain comprises an FcRγ transmembrane domain. In some embodiments, the transmembrane domain comprises an FcRα transmembrane domain. In some embodiments, the transmembrane domain comprises an FcRβ transmembrane domain. In some embodiments, the transmembrane domain comprises an FGRε transmembrane domain. In some embodiments, the transmembrane domain comprises a transmembrane domain from a syntaxin, such as syntaxin 3 or syntaxin 4 or syntaxin 5. In some embodiments, the transmembrane domain oligomerizes with a transmembrane domain of an endogenous receptor when the CFP is expressed in a cell. In some embodiments, the transmembrane domain oligomerizes with a transmembrane domain of an exogenous receptor when the CFP is expressed in a cell. In some embodiments, the transmembrane domain dimerizes with a transmembrane domain of an endogenous receptor when the CFP is expressed in a cell. In some embodiments, the transmembrane domain dimerizes with a transmembrane domain of an exogenous receptor when the CFP is expressed in a cell. In some embodiments, the transmembrane domain is derived from a protein that is different than the protein from which the intracellular signaling domain is derived. In some embodiments, the transmembrane domain is derived from a protein that is different than the protein from which the extracellular domain is derived. In some embodiments, the transmembrane domain comprises a transmembrane domain of a phagocytic receptor. In some embodiments, the transmembrane domain and the extracellular domain are derived from the same protein. In some embodiments, the transmembrane domain is derived from the same protein as the intracellular signaling domain. In some embodiments, the recombinant nucleic acid encodes a DAP12 recruitment domain. In some embodiments, the transmembrane domain comprises a transmembrane domain that oligomerizes with DAP12.

In some embodiments, the transmembrane domain is at least 12, 13, 14, 15, 16, 17, 18, 19, 20, 21, 22, 23, 24, 25, 26, 27, 28, 29, 30, 31 or 32 amino acids in length. In some embodiments, the transmembrane domain is at most 12, 13, 14, 15, 16, 17, 18, 19, 20, 21, 22, 23, 24, 25, 26, 27, 28, 29, 30, 31 or 32 amino acids in length.

In some embodiments, the intracellular signaling domain comprises an intracellular signaling domain derived from a phagocytic receptor. In some embodiments, the intracellular signaling domain comprises an intracellular signaling domain derived from a phagocytic receptor other than a phagocytic receptor selected from Megf10, MerTk, FcRα, or Bai1. In some embodiments, the intracellular signaling domain comprises an intracellular signaling domain derived from a phagocytic receptor selected from the group consisting of TNFR1, MDA5, CD40, lectin, dectin 1, CD206, scavenger receptor A1 (SRA1), MARCO, CD36, CD163, MSR1, SCARA3, COLEC12, SCARA5, SCARB1, SCARB2, CD68, OLR1, SCARF1, SCARF2, CXCL16, STAB1, STAB2, SRCRB4D, SSC5D, CD205, CD207, CD209, RAGE, CD14, CD64, F4/80, CCR2, CX3CR1, CSF1R, Tie2, HuCRIg(L), CD64, CD32a, CD16a, CD89, Fc-alpha receptor I, CR1, CD35, CD3C, CR3, CR4, Tim-1, Tim-4 and CD169. In some embodiments, the intracellular signaling domain comprises a PI3K recruitment domain. In some embodiments, the intracellular signaling domain comprises an intracellular signaling domain derived from a scavenger receptor. In some embodiments, the intracellular domain comprises a CD47 inhibition domain. In some embodiments, the intracellular domain comprises a Rac inhibition domain, a Cdc42 inhibition domain or a GTPase inhibition domain. In some embodiments, the Rac inhibition domain, the Cdc42 inhibition domain or the GTPase inhibition domain inhibits Rac, Cdc42 or GTPase at a phagocytic cup of a cell expressing the PFP. In some embodiments, the intracellular domain comprises an F-actin disassembly activation domain, a ARHGAP12 activation domain, a ARHGAP25 activation domain or a SH3BP1 activation domain. In some embodiments, the intracellular domain comprises a phosphatase inhibition domain. In some embodiments, the intracellular domain comprises an ARP2/3 inhibition domain. In some embodiments, the intracellular domain comprises at least one ITAM domain. In some embodiments, the intracellular domain comprises at least 1, 2, 3, 4, 5, 6, 7, 8, 9, 10, or more ITAM domains. In some embodiments, the intracellular domain comprises at least one ITAM domain select from an ITAM domain of CD3 zeta, CD3 epsilon, CD3 gamma, CD3 delta, Fc epsilon receptor 1 chain, Fc epsilon receptor 2 chain, Fc gamma receptor 1 chain, Fc gamma receptor 2a chain, Fc gamma receptor 2b 1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d, functional fragments thereof, and amino acid sequences thereof having at least one but not more than 20 modifications thereto. In some embodiments, the at least one ITAM domain comprises a Src-family kinase phosphorylation site. In some embodiments, the at least one ITAM domain comprises a Syk recruitment domain. In some embodiments, the intracellular domain comprises an F-actin depolymerization activation domain. In some embodiments, the intracellular domain lacks enzymatic activity.

In some embodiments, the CFP encoded by the recombinant nucleic acid can further comprise a transmembrane domain, such as a transmembrane domain derived from CD2, CD8, CD28, CD68, a phagocytic receptor, a scavenger receptor or an integrin receptor. In some embodiments, a CFP encoded by the recombinant nucleic acid further comprises an intracellular domain comprising an intracellular signaling domain, such as an intracellular signaling domain derived from a phagocytic receptor, a scavenger receptor or an integrin receptor. For example, the intracellular domain can comprise one or more intracellular signaling domains derived from a phagocytic receptor, a scavenger receptor or an integrin receptor. For example, the intracellular domain can comprise one or more intracellular signaling domains that promote phagocytic activity, inflammatory response, nitric oxide production, integrin activation, enhanced effector cell migration (e.g., via chemokine receptor expression), antigen presentation, and/or enhanced cross presentation. In some embodiments, the CFP is a phagocytic receptor fusion protein (PFP). In some embodiments, the CFP is a phagocytic scavenger receptor fusion protein (PFP). In some embodiments, the CFP is an integrin receptor fusion protein (IFP). In some embodiments, the CFP is an inflammatory receptor fusion protein. In some embodiments, a CFP encoded by the recombinant nucleic acid further comprises an intracellular domain comprising a recruitment domain. For example, the intracellular domain can comprise one or more PI3K recruitment domains, caspase recruitment domains or caspase activation and recruitment domains (CARDs).

In some embodiments, the intracellular domain does not comprise a domain derived from a CD3 zeta intracellular domain. In some embodiments, the intracellular domain does not comprise a domain derived from a MerTK intracellular domain. In some embodiments, the intracellular domain does not comprise a domain derived from a TLR4 intracellular domain. In some embodiments, the intracellular domain comprises a CD47 inhibition domain. In some embodiments, the intracellular signaling domain comprises a domain that activates integrin, such as the intracellular region of PSGL-1

In some embodiments, the intracellular signaling domain comprises a domain that activates Rap1 GTPase, such as that from EPAC and C3G. In some embodiments, the intracellular signaling domain is derived from paxillin. In some embodiments, the intracellular signaling domain activates focal adhesion kinase. In some embodiments, the intracellular signaling domain is derived from a single phagocytic receptor. In some embodiments, the intracellular signaling domain is derived from a single scavenger receptor. In some embodiments, the intracellular domain comprises a phagocytosis enhancing domain.

In some embodiments, the intracellular domain comprises a pro-inflammatory signaling domain. In some embodiments, the pro-inflammatory signaling domain comprises a kinase activation domain or a kinase binding domain. In some embodiments, the pro-inflammatory signaling domain comprises an IL-1 signaling cascade activation domain. In some embodiments, the pro-inflammatory signaling domain comprises an intracellular signaling domain derived from TLR3, TLR4, TLR7, TLR 9, TRIF, RIG-1, MYD88, MAL, IRAK1, MDA-5, an IFN-receptor, STING, an NLRP family member, NLRP1-14, NOD1, NOD2, Pyrin, AIM2, NLRC4, FCGR3A, FCERIG, CD40, Tank1-binding kinase (TBK), a caspase domain, a procaspase binding domain or any combination thereof.

In some embodiments, the intracellular domain comprises a signaling domain, such as an intracellular signaling domain, derived from a connexin (Cx) protein. For example, the intracellular domain can comprise a signaling domain, such as an intracellular signaling domain, derived from Cx43, Cx46, Cx37, Cx40, Cx33, Cx50, Cx59, Cx62, Cx32, Cx26, Cx31, Cx30.3, Cx31.1, Cx30, Cx25, Cx45, Cx47, Cx31.3, Cx36, Cx31.9, Cx39, Cx40.1 or Cx23. For example, the intracellular domain can comprise a signaling domain, such as an intracellular signaling domain, derived from Cx43.

In some embodiments, the intracellular domain comprises a signaling domain, such as an intracellular signaling domain, derived from a SIGLEC protein. For example, the intracellular domain can comprise a signaling domain, such as an intracellular signaling domain, derived from Siglec-1 (Sialoadhesin), Siglec-2 (CD22), Siglec-3 (CD33), Siglec-4 (MAG), Siglec-5, Siglec-6, Siglec-7, Siglec-8, Siglec-9, Siglec-10, Siglec-11, Siglec-12, Siglec-13, Siglec-14, Siglec-15, Siglec-16 or Siglec-17.

In some embodiments, the intracellular domain comprises a signaling domain, such as an intracellular signaling domain, derived from a C-type lectin protein. For example, the intracellular domain can comprise a signaling domain, such as an intracellular signaling domain, derived from a mannose receptor protein. For example, the intracellular domain can comprise a signaling domain, such as an intracellular signaling domain, derived from an asialoglycoprotein receptor protein. For example, the intracellular domain can comprise a signaling domain, such as an intracellular signaling domain, derived from macrophage galactose-type lectin (MGL), DC-SIGN (CLEC4L), Langerin (CLEC4K), Myeloid DAP12 associating lectin (MDL)-1 (CLEC5A), a DC associated C type lectin 1 (Dectin1) subfamily protein, dectin 1/CLEC7A, DNGR1/CLEC9A, Myeloid C type lectin like receptor (MICL) (CLEC12A), CLEC2 (CLEC1B), CLEC12B, a DC immunoreceptor (DCIR) subfamily protein, DCIR/CLEC4A, Dectin 2/CLEC6A, Blood DC antigen 2 (BDCA2) (CLEC4C), Mincle (macrophage inducible C type lectin) (CLEC4E), a NOD-like receptor protein, NOD-like receptor MHC Class II transactivator (CIITA), IPAF, BIRC1, a RIG-I-like receptor (RLR) protein, RIG-I, MDA5, LGP2, NAIP5/Bircle, an NLRP protein, NLRP1, NLRP2, NLRP3, NLRP4, NLRP5, NLRP6, NLRP7, NLRP89, NLRP9, NLRP10, NLRP11, NLRP12, NLRP13, NLRP14, an NLR protein, NOD1 or NOD2, or any combination thereof.

In some embodiments, the intracellular domain comprises a signaling domain, such as an intracellular signaling domain, derived from a cell adhesion molecule. For example, the intracellular domain can comprise a signaling domain, such as an intracellular signaling domain, derived from an IgCAMs, a cadherin, an integrin, a C-type of lectin-like domains protein (CTLD) and/or a proteoglycan molecule. For example, the intracellular domain can comprise a signaling domain, such as an intracellular signaling domain, derived from an E-cadherin, a P-cadherin, an N-cadherin, an R-cadherin, a B-cadherin, a T-cadherin, or an M-cadherin. For example, the intracellular domain can comprise a signaling domain, such as an intracellular signaling domain, derived from a selectin, such as an E-selectin, an L-selectin or a P-selectin.

In some embodiments, the CFP does not comprise a full length intracellular signaling domain. In some embodiments, the intracellular domain is at least 5, 10, 20, 30, 40, 50, 60, 70, 80, 90, 100, 150, 200, 300, 300, 400, or 500 amino acids in length. In some embodiments, the intracellular domain is at most 10, 20, 30, 40, 50, 60, 70, 80, 90, 100, 150, 200, 300, 300, 400, or 500 amino acids in length.

In some embodiments, the recombinant nucleic acid encodes an FcRα chain extracellular domain, an FcRα chain transmembrane domain and/or an FcRα chain intracellular domain. In some embodiments, the recombinant nucleic acid encodes an FcRβ chain extracellular domain, an FcRβ chain transmembrane domain and/or an FcRβ chain intracellular domain. In some embodiments, the FcRα chain or the FcRβ chain forms a complex with FcRγ when expressed in a cell. In some embodiments, the FcRα chain or FcRβ chain forms a complex with endogenous FcRγ when expressed in a cell. In some embodiments, the FcRα chain or the FcRβ chain does not incorporate into a cell membrane of a cell that does not express FcRγ. In some embodiments, the CFP does not comprise an FcRα chain intracellular signaling domain. In some embodiments, the CFP does not comprise an FcRβ chain intracellular signaling domain. In some embodiments, the recombinant nucleic acid encodes a TREM extracellular domain, a TREM transmembrane domain and/or a TREM intracellular domain. In some embodiments, the TREM is TREM1, TREM 2 or TREM 3.

In some embodiments, the intracellular signaling subunit comprising an intracellular signaling domain having tyrosine residues comprise at least one ITAM domain. In some embodiments, the intracellular signaling subunit comprises more than one ITAM domains. In some embodiments, the at least one ITAM domain select from a group CD3 zeta TCR subunit, CD3 epsilon TCR subunit, CD3 gamma TCR subunit, CD3 delta TCR subunit, TCR zeta chain, Fc epsilon receptor 1 chain, Fc epsilon receptor 2 chain, Fc gamma receptor 1 chain, Fc gamma receptor 2a chain, Fc gamma receptor 2b 1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d, functional fragments thereof, and amino acid sequences thereof having at least one but not more than 20 modifications thereto. In some embodiments, the at least one ITAM domain comprises a Src-family kinase phosphorylation site. In some embodiments, the at least one ITAM domain comprises a Syk recruitment domain.

In some embodiments of the various aspects described herein, the intracellular signaling subunit further comprises a DAP12 recruitment domain. In some embodiments, the intracellular domain comprises at least 1, 2, 3, 4, 5, 6, 7, 8, 9, or 10 ITAM domains. In some embodiments, the intracellular signaling subunit further comprises a pro-inflammatory signaling domain comprising an IL-1 signaling cascade activation domain. In some embodiments, the pro-inflammatory signaling domain comprises an intracellular signaling domain derived from TLR3, TLR4, TLR7, TLR 9, TRIF, RIG-1, MYD88, MAL, IRAK1, MDA-5, an IFN-receptor, an NLRP family member, NLRP1-14, NOD1, NOD2, Pyrin, AIM2, NLRC4, FCGR3A, FCERIG, CD40, a caspase domain or a procaspase binding domain or any combination thereof.

In some embodiments, the intracellular signaling domain further comprises a domain that activate integrin such as the intracellular region of PSGL-1.

In some embodiments, the intracellular signaling domain further comprises a domain that activate Rap1 GTPase, such as that from EPAC and C3 G. In some embodiments, the intracellular signaling domain further comprises a domain from paxillin.

In some embodiments, the intracellular signaling domain activates focal adhesion kinase.

Provided herein are myeloid cell-specific expression constructs encoding a recombinant protein, for example, a chimeric fusion protein (CFP). In some embodiments, the CFP comprises (i) a sequence encoding an extracellular antigen binding domain that binds to a target antigen, where the binding domain comprises a target-specific antibody or a fragment thereof, e.g., an scFv, for example, an scFv that binds to a target antigen expressed on a cancer cell, (ii) a sequence encoding a transmembrane domain capable of dimerizing with an Fc-gamma receptor transmembrane domain upon expression in a cell, and (iii) a sequence encoding one or more intracellular domains comprising a signaling domain that can activate intracellular signal transduction for phagocytosis activation, inflammatory cytokine secretion and/or immune activation in the cell expressing the construct; wherein the expression constructs is a polynucleotide, encapsulated in a lipid nanoparticle for delivery. In some embodiments, the polynucleotide is an mRNA.

Provided herein are myeloid cell-specific expression constructs encoding a CFP, wherein the CFP comprises (i) a sequence encoding an extracellular antigen binding domain that binds to a target antigen, e.g., a target antigen expressed on a cancer cell, for example, CD5, HER2, TROP2, GPC3, GP75, CD19, CD7, CD22 or any other conceivable target antigen, (ii) a sequence encoding a transmembrane domain capable of dimerizing with an Fc-gamma receptor transmembrane domain upon expression in a cell, for example, a CD89 TMD, a CD16 TMD, a CD64 TMD or a CD32a TMD, and (iii) a sequence encoding one or more intracellular domains comprising a signaling domain that can activate intracellular signal transduction for phagocytosis activation, inflammatory cytokine secretion and/or immune activation in the cell expressing the construct; wherein the expression constructs is a polynucleotide, wherein the expression construct is encapsulated in a lipid nanoparticle for delivery. In some embodiments, the polynucleotide is an mRNA.

In some embodiments the target protein is GPC3. In some embodiments, the antigen binding domain comprises an anti-GPC3 antibody or binding fragment thereof, wherein the antigen binding domain comprises a heavy chain variable domain (VH) comprising a heavy chain complementarity determining region 3 (HC CDR3) of any one of the sequences selected from the group consisting of ATACADTTQYAYDY (SEQ ID NO: 2), ATACADTTLYEYDY (SEQ ID NO: 3), ATACVDTTQYEYDY (SEQ ID NO: 4), ATACADATQHEYDY (SEQ ID NO: 5), ATACADTTQYDYDY (SEQ ID NO: 6), ATACADTTQYEYDY (SEQ ID NO: 7), ATACADTTHYEYDY (SEQ ID NO: 8), ATACVITTLYEYDY (SEQ ID NO: 9), ATACAETTLYEYDY (SEQ ID NO: 10), ATACADTTQHEYDY (SEQ ID NO: 11), ATACVDTTHYEYDY (SEQ ID NO: 12), ATACASTTLYEYDY (SEQ ID NO: 13), ATACVVTTLYEYDY (SEQ ID NO: 14), ATACGGATGPYDY (SEQ ID NO: 15), ATACAGAIGPYDY (SEQ ID NO: 16), ATACVVVGDQNDY (SEQ ID NO: 17), ATACVVVGDRNDY (SEQ ID NO: 18), ATDCAGGTSTPYDY (SEQ ID NO: 19), ATDCAGGTATPYDY (SEQ ID NO: 20), ATACVVADRNEYDY (SEQ ID NO: 21), ATSCVVVTKNEYDY (SEQ ID NO: 22), ATACSGLTHEYDY (SEQ ID NO: 23), ATTCSGLTHEYDY (SEQ ID NO: 24), ATACANWSSLGPYDY (SEQ ID NO: 25), ATACANWSTLGPYDY (SEQ ID NO: 26), ATACSDPRVYEYDY (SEQ ID NO: 27), ATTCASPEKYEYDY (SEQ ID NO: 28), ATHCGGTSWGTSYDY (SEQ ID NO: 29), ATHCGGSSWSNEYDY (SEQ ID NO: 30), YARYSGRTY (SEQ ID NO: 31), ASSAWPAGPKHQVEYDY (SEQ ID NO: 32), ATACGSLVGMYDY (SEQ ID NO: 33), ATACGSAVHEYDY (SEQ ID NO: 34), ATDCVGFGSNWFDY (SEQ ID NO: 35), ATACASPVIYEYDY (SEQ ID NO: 36), ATDCAGGVGHEYDY (SEQ ID NO: 37), ATDCSLHGSDYPYDY (SEQ ID NO: 38) and AVRIYSGSFDNTLAYDY (SEQ ID NO: 39). In some embodiments, the VH of the anti-GPC3 antibody or binding fragment thereof further comprises a heavy chain complementarity determining region 1 (HC CDR1) of any one of the sequences selected from the group consisting of GFPLAYYA (SEQ ID NO: 40), GFSLDYYA (SEQ ID NO: 41), GFPLDYYA (SEQ ID NO: 42), GFTLDYYA (SEQ ID NO: 43), GFSLNYYA (SEQ ID NO: 44), GFTLAYYA (SEQ ID NO: 45), GFTLGYYA (SEQ ID NO: 46), GFPLNYYA (SEQ ID NO: 47), GFPLHYYA (SEQ ID NO: 48), GFSLGYYA (SEQ ID NO: 49), GFPLGYYA (SEQ ID NO: 50), GFPLEYYA (SEQ ID NO: 51), GSDFRADA (SEQ ID NO: 52), GRTFSSYG (SEQ ID NO: 53), GFSLAYYA (SEQ ID NO: 54) and GLTFRSVG (SEQ ID NO: 55). In some embodiments, the VH of the anti-GPC3 antibody or binding fragment thereof further comprises a heavy chain complementarity determining region 2 (HC CDR2) of any one of the sequences selected from the group consisting of sequences: ISNSDGST (SEQ ID NO: 56), ISASDGST (SEQ ID NO: 57), ISSSDGST (SEQ ID NO: 58), ISSSDGNT (SEQ ID NO: 59), ISSADGST (SEQ ID NO: 60), ISSSGGST (SEQ ID NO: 61), ISSGDGST (SEQ ID NO: 62), ISAGDGNT (SEQ ID NO: 63), ISSSDDST (SEQ ID NO: 64), ISSNDGST (SEQ ID NO: 65), ISSPDGST (SEQ ID NO: 66), ISSRTGGT (SEQ ID NO: 67), ISAGDGSST (SEQ ID NO: 68), ISSSDGSSSDGNT (SEQ ID NO: 69), ISSGDGNT (SEQ ID NO: 70), ISSGDGKT (SEQ ID NO: 71), ISSSDGGT (SEQ ID NO: 72), ISSRTGST (SEQ ID NO: 73), ISSRTGNT (SEQ ID NO: 74), ISSSDGHSST (SEQ ID NO: 75), ISSSSDGNT (SEQ ID NO: 76), ISASNGNT (SEQ ID NO: 77), ISSGSDGNT (SEQ ID NO: 78), ISASDGNT (SEQ ID NO: 79), IDSITSI (SEQ ID NO: 80), ISWSGGSTIAASVGST (SEQ ID NO: 81), ISSSDGSDGNT (SEQ ID NO: 82) and ASPSGVIT (SEQ ID NO: 83). In some embodiments, the VH of the anti-GPC3 antibody or binding fragment thereof comprises with 70-100% sequence identity to any one of the sequences selected from the group consisting of

(SEQ ID NO: 84)
QVQLQESGGGLVHSGGSLRLSCAASGFPLAYYAIGWFRQAPGKEREGVSCISSSDGNTYYADAV
KGRFTISRDNAKNTVYLQMNSLKPEDTAVYYCATACADTTQHEYDYWGQGTQVTVSS,
(SEQ ID NO: 85)
QVQLQESGGGLVHSGGSLRLSCAASGFPLDYYAIGWFRQAPGKEREGVSCISSADGSTYYADSV
KGRFTISRDNAKNTVYLQMNSLGPEDTAVYYCATACADTTQYDYDYWGQGTQVTVSS,
(SEQ ID NO: 86)
QVQLQESGGGLVHSGGSLRLSCAASGFTLDYYAIGWFRRAPGKEREGVSCISSGDGKTYYADSV
KGRFTISRDNAKNTVYLQMNSLKPEDTAVYYCATACAGAIGPYDYWGQGTQVTVSS,
(SEQ ID NO: 87)
QVQLQESGGGLVPPGGSLRLSCAASGFPLDYYAIGWFRQAPGKEREGVSCISSADGSTYYADSV
KGRFTISRDNAKNTVYLQMNSLGPEDTAVYYCATACADTTQYDYDYWGQGTQVTVSS,
(SEQ ID NO: 88)
QVQLQESGGGLVQAGGSLRLSCAASGFSLGYYAIGWFRQAPGKEREGVSCISSSDGHSSTYYAD
SVKGRFTISRDNAKNTVYLQMNNLKPEDTAVYYCATDCAGGTATPYDYWGQGTQVTVSS,
(SEQ ID NO: 89)
QVQLQESGGGLVQAGGSLRLSCAASGRTFSSYGMGWFRQAPGKEREFVAAISWSGGSTYYADS
VKGRFTISRDNAKNTVYLQMNSLKPEDTAVYYCASSAWPAGPKHQVEYDYWGQGTQVTVSS,
(SEQ ID NO: 90)
QVQLQESGGGLVQAGGSLRLSCTASGFSLDYYAIGWFRQAPGKEREGVACISSRTGSTYYADSV
KGRFTISRDNAKNTVALQMNSLKPEDTAVYYCATACVVVGDQNDYWGQGTQVTVSS,
(SEQ ID NO: 91)
QVQLQESGGGLVQDGGSLRLSCAASGFPLAYYAIGWFRQAPGKEREGVSCISASDGSTYYADSV
KGRFTISRDNAKNTVYLQMNSLKPEDTAVYYCATACAETTLYEYDYWGQGTQVTVSS,
(SEQ ID NO: 92)
QVQLQESGGGLVQPGESLRLSCAASGFPLAYYAIGWFRQAPGKEREGVSCISSSDGSTYYADSV
KGRFTISRDNAKNTVYLQMNSLKPEDTAVYYCATACANWSTLGPYDYWGQGTQVTVSS,
(SEQ ID NO: 93)
QVQLQESGGGLVQPGESLRLSCAASGFTLAYYAIGWFRQAPGKEREGVSCISSSDGNTYYADSV
KGRFTISRDNAKNTVYLQMNRLKPEDTAVYYCATACADTTQYEYDYWGQGTQVTVSS,
(SEQ ID NO: 94)
QVQLQESGGGLVQPGGSLKLSCAASGSDFRADAMGWYRQAPGKEREPVAIDSITSIYYVDSVEG
RFTISRDNTKNTVYLQMTSLKPEDTAVYYCYARYSGRTYWGRGTQVTVSS,
(SEQ ID NO: 95)
QVQLQESGGGLVQPGGSLRLSCAASGFPLAYYAIGWFRQAPGKEREGVSCISASDGSTYYADSV
KGRFTISRDNAKNTVYLQMNSLRPEDTAVYYCATACADTTLYEYDYWGQGTQVTVSS,
(SEQ ID NO: 96)
QVQLQESGGGLVQPGGSLRLSCAASGFPLAYYAIGWFRQAPGKEREGVSCISSSDGNTYYADAV
KGRFAISRDNAKNTVYLQMNSLKPEDTAVYYCATACSDPRVYEYDYWGQGTQVTVSS,
(SEQ ID NO: 97)
QVQLQESGGGLVQPGGSLRLSCAASGFPLAYYAIGWFRQAPGKEREGVSCISSSDGNTYYADAV
KGRFTISRDNAKNTVYLQMNSLKPEDTAVYYCATACADTTQHEYDYWGQGTQVTVSS,
(SEQ ID NO: 98)
QVQLQESGGGLVQPGGSLRLSCAASGFPLAYYAIGWFRQAPGKEREGVSCISSSDGNTYYADAV
KGRFTISRDNAKNTVYLQMNSLKPEDTAVYYCATACVDTTHYEYDYWGQGTQVTVSS,
(SEQ ID NO: 99)
QVQLQESGGGLVQPGGSLRLSCAASGFPLAYYAIGWFRQAPGKEREGVSCISSSDGNTYYADSV
KGRFTISRDNAKNTVYLQMNSLKPEDTAVYYCATACADATQHEYDYWGQGTQVTVSS,
(SEQ ID NO: 100)
QVQLQESGGGLVQPGGSLRLSCAASGFPLAYYAIGWFRQAPGKEREGVSCISSSDGSTYYADSV
KGRFTISRDNAKNTVYLQMNSLGPEDTAVYYCATACADTTQYDYDYWGQGTQVTVSS,
(SEQ ID NO: 101)
QVQLQESGGGLVQPGGSLRLSCAASGFPLAYYAIGWFRQAPGKEREGVSCISSSDGSTYYADSV
KGRFTISRDNAKNTVYLQMNSLKPEDTAVYYCATACADTTQYEYDYWGQGTQVTVSS,
(SEQ ID NO: 102)
QVQLQESGGGLVQPGGSLRLSCAASGFPLAYYAIGWFRQAPGKEREGVSCISSSDGSTYYADSV
KGRFTISRDNAKNTVYLQMNSLKPEDTAVYYCATACGGATGPYDYWGQGTQVTVSS,
(SEQ ID NO: 103)
QVQLQESGGGLVQPGGSLRLSCAASGFPLAYYAIGWFRRAPGKEREGVSCISSSDGNTYYADAV
KGRFTISRDNAKNTVYLQMNSLKPEDTAVYYCATACADTTQHEYDYWGQGTQVTVSS,
(SEQ ID NO: 104)
QVQLQESGGGLVQPGGSLRLSCAASGFPLDYYAIGWFRQAPGKEREGVSCISAGDGSSTYYADS
VKGRFTISRDNAKNTVYLQMNSLKPEDTAVYYCATACASTTLYEYDYWGQGTQVTVSS,
(SEQ ID NO: 105)
QVQLQESGGGLVQPGGSLRLSCAASGFPLDYYAIGWFRQAPGKEREGVSCISSADGSTYYADSV
KGRFTISRDNAKNAVYLQMNSLGPEDTAVYYCATACADTTQYDYDYWGQGTQVTVSS,
(SEQ ID NO: 106)
QVQLQESGGGLVQPGGSLRLSCAASGFPLDYYAIGWFRQAPGKEREGVSCISSADGSTYYADSV
KGRFTISRDNAKNTVYLQMNSLGPEDTAVYYCATACADTTQYDYDYWGQGTQVTVSS,
(SEQ ID NO: 107)
QVQLQESGGGLVQPGGSLRLSCAASGFPLDYYAIGWFRQAPGKEREGVSCISSADGSTYYADSV
KGRFTISRDNAKNTVYLQMNSLKPEDTAVYYCATACVDTTQYEYDYWGQGTQVTVSS,
(SEQ ID NO: 108)
QVQLQESGGGLVQPGGSLRLSCAASGFPLDYYAIGWFRQAPGKEREGVSCISSPDGSTYYADSV
KGRFTISRDNAKNTVYLQMNSLKPEDTAVYYCATACVDTTQYEYDYWGQGTQVTVSS,
(SEQ ID NO: 109)
QVQLQESGGGLVQPGGSLRLSCAASGFPLDYYAIGWFRQAPGKEREGVSCISSSDGSDGNTYYA
DSVKGRFTISRDNAKNTVYLQMNSLKPEDTAVYYCATDCSLHGSDYPYDYWGQGTQVTVSS,
(SEQ ID NO: 110)
QVQLQESGGGLVQPGGSLRLSCAASGFPLDYYAIGWFRQAPGKEREGVSCISSSDGSTYYADSV
KGRFTISRDNAKNTVYLQMNSLKPEDTAVYYCATACADTTQYEYDYWGQGTQVTVSS,
(SEQ ID NO: 111)
QVQLQESGGGLVQPGGSLRLSCAASGFPLEYYAIGWFRQAPGKEREGVSCISSSDGSTYYADSV
KGRFTISRDNAKNTVYLQMNSLKPEDTAVYYCATACSDPRVYEYDYWGQGTQVTVSS,
(SEQ ID NO: 112)
QVQLQESGGGLVQPGGSLRLSCAASGFPLGYYAIGWFRQAPGKEREGVSCISSSDDSTYYADSV
KGRFTISRDNDKNTVYLQMNSLKPEDTAVYYCATDCAGGTSTPYDYWGQGTQVTVSS,
(SEQ ID NO: 113)
QVQLQESGGGLVQPGGSLRLSCAASGFPLHYYAIGWFRQAPGKEREGVSCISSGDGSTYYADSV
KGRFTISRDNAKNTVYLQMNSLKPEDTAVYYCATSCVVVTKNEYDYWGQGTQVTVSS,
(SEQ ID NO: 114)
QVQLQESGGGLVQPGGSLRLSCAASGFPLHYYAIGWFRQAPGKEREGVSCISSSDGSTYYADSV
KGRFTISRDNAKNTVYLQMNSLKPEDTAVYYCATACGGATGPYDYWGQGTQVTVSS,
(SEQ ID NO: 115)
QVQLQESGGGLVQPGGSLRLSCAASGFPLHYYAIGWFRQAPGKEREGVSCISSSDGSTYYADSV
KGRFTISRDNAKNTVYLQMNSLKPEDTAVYYCATACVVADRNEYDYWGQGTQVTVSS,
(SEQ ID NO: 116)
QVQLQESGGGLVQPGGSLRLSCAASGFPLHYYAIGWFRQAPGKEREGVSCISSSDGSTYYADSV
KGRFTISRDNAKNTVYLQMNSLRPEDTAVYYCATACVVADRNEYDYWGQGTQVTVSS,
(SEQ ID NO: 117)
QVQLQESGGGLVQPGGSLRLSCAASGFPLNYYAIGWFRQAPGKEREGVSCISASDGNTYYADSV
KGRFTISRDNAKNTVYLQMNSLKPEDTAVYYCATTCASPEKYEYDYWGQGTQVTVSS,
(SEQ ID NO: 118)
QVQLQESGGGLVQPGGSLRLSCAASGFPLNYYAIGWFRQAPGKEREGVSCISSSDGSTYYADSV
KGRFIISRDNAKNTVYLQMNSLKPEDTAVYYCATACGGATGPYDYWGQGTQVTVSS,
(SEQ ID NO: 119)
QVQLQESGGGLVQPGGSLRLSCAASGFPLNYYAIGWFRQAPGKEREGVSCISSSDGSTYYADSV
KGRFTISRDNAKNTVYLQMNSLKPEDTAVYYCATACGSAVHEYDYWGQGTQVTVSS,
(SEQ ID NO: 120)
QVQLQESGGGLVQPGGSLRLSCAASGFSLAYYAIGWFRQAPGKEREGVSCIAASVGSTYYADSV
KGRFTISRDDAKNTVYLQMNSLKPEDTAVYYCATDCAGGVGHEYDYWGQGTQVTVSS,
(SEQ ID NO: 121)
QVQLQESGGGLVQPGGSLRLSCAASGFSLDYYAIGWFRQAPGKEREGVSCISSSDGSTYYADSV
KGRFTISRDNAKNAVYLQMNSLKPEDTAVYYCATACGGATGPYDYWGQGTQVTVSS,
(SEQ ID NO: 122)
QVQLQESGGGLVQPGGSLRLSCAASGFSLDYYAIGWFRQAPGKEREGVSCISSSDGSTYYADSV
KGRFTISRDNAKNAVYLQMNSLKPEDTAVYYCATACVDTTQYEYDYWGQGTQVTVSS,
(SEQ ID NO: 123)
QVQLQESGGGLVQPGGSLRLSCAASGFSLDYYAIGWFRQAPGKEREGVSCISSSDGSTYYADSV
KGRFTISRDNAKNTVYLQMNSLKPEDTAVYYCATDCAGGTSTPYDYWGQGTQVTVSS,
(SEQ ID NO: 124)
QVQLQESGGGLVQPGGSLRLSCAASGFSLNYYAIGWFRQAPGKEREGVSCISAGDGNTYYADS
VKGRFTISRDNAANTVSLQMDSLKPEDTAVYYCATACVITTLYEYDYWGQGTQVTVSS,
(SEQ ID NO: 125)
QVQLQESGGGLVQPGGSLRLSCAASGFTLAYYAIGWFRQAPGKEREGVSCISSSDGSTYYADSV
KGRFTISRDNAKNTVYLQMNSLKPEDTAVYYCATACADTTQHEYDYWGQGTQVTVSS,
(SEQ ID NO: 126)
QVQLQESGGGLVQPGGSLRLSCAASGFTLAYYAIGWFRQAPGKEREGVSCISSSDGSTYYADSV
KGRFTISRDNAKNTVYLQMNSLKPEDTAVYYCATACADTTQYEYDYWGQGTQVTVSS,
(SEQ ID NO: 127)
QVQLQESGGGLVQPGGSLRLSCAASGFTLDYYAIGWFRQAPGKEREGVACISSSDGSTYYADSV
KGRFTISRDNAKNTVYLQMNSLKPEDTAVYYCATACGGATGPYDYWGQGTQVTVSS,
(SEQ ID NO: 128)
QVQLQESGGGLVQPGGSLRLSCAASGFTLDYYAIGWFRQAPGKEREGVACISSSDGSTYYADSV
KGRFTISRDNAKNTVYLQMNSLKPQDTAVYYCATACGSLVGMYDYWGQGTQVTVSP
(SEQ ID NO: 129)
QVQLQESGGGLVQPGGSLRLSCAASGFTLDYYAIGWFRQAPGKEREGVSCISASDGNTYYADS
VKGRFTISRDNAKNTVYLQMNSLKPEDTAVYYCATTCASPEKYEYDYWGQGTQVTVSS,
(SEQ ID NO: 130)
QVQLQESGGGLVQPGGSLRLSCAASGFTLDYYAIGWFRQAPGKEREGVSCISASNGNTYYADS
VKGRFTISRDSAKNTVYLQMNSLKPEDTAVYYCATTCSGLTHEYDYWGQGTQVTVSS,
(SEQ ID NO: 131)
QVQLQESGGGLVQPGGSLRLSCAASGFTLDYYAIGWFRQAPGKEREGVSCISSGDGNTYYADS
VKGRFTISRDNAKNTVYLQMNSLKPEDTAVYYCATACGGATGPYDYWGQGTQVTVSS,
(SEQ ID NO: 132)
QVQLQESGGGLVQPGGSLRLSCAASGFTLDYYAIGWFRQAPGKEREGVSCISSGDGSTYYADSV
KGRFTISRDNAKNTVYLQMNSLKPEDTAVYYCATHCGGSSWSNEYDYWGQGTQVTVSS,
(SEQ ID NO: 133)
QVQLQESGGGLVQPGGSLRLSCAASGFTLDYYAIGWFRQAPGKEREGVSCISSNDGSTYYADSV
KGRFTISRDNAKNTVYLQMNSLKPEDTAVYYCATACADTTQHEYDYWGQGTQVTVSS,
(SEQ ID NO: 134)
QVQLQESGGGLVQPGGSLRLSCAASGFTLDYYAIGWFRQAPGKEREGVSCISSSDGGTYYADSV
KGRFTISRDNAKNTVYLQMNSLKPEDTAVYYCATACGGATGPYDYWGQGTQVTVSS,
(SEQ ID NO: 135)
QVQLQESGGGLVQPGGSLRLSCAASGFTLDYYAIGWFRQAPGKEREGVSCISSSDGSSSDGNTY
YADSVKGRFTISRDNAKNTVYLQMNNLKPEDTAVYYCATACVVTTLYEYDYWGQGTQVTVS
(SEQ ID NO: 136)
QVQLQESGGGLVQPGGSLRLSCAASGFTLDYYAIGWFRQAPGKEREGVSCISSSDGSTYYADSV
KGRFTISRDNAKNTVYLQMNSLKPEDTAVYYCATACADTTQYEYDYWGQGTQVTVSP
(SEQ ID NO: 137)
QVQLQESGGGLVQPGGSLRLSCAASGFTLDYYAIGWFRQAPGKEREGVSCISSSDGSTYYADSV
KGRFTISRDNAKNTVYLQMNSLKPEDTAVYYCATACGGATGPYDYWGQGTQVTVSS,
(SEQ ID NO: 138)
QVQLQESGGGLVQPGGSLRLSCAASGFTLDYYAIGWFRQAPGKEREGVSCISSSGGSTYYADSV
KGRFTISRDNAKNTVYLQMNMLKPEDTAVYYCATACADTTQYEYDYWGQGTQVTVSS,
(SEQ ID NO: 139)
QVQLQESGGGLVQPGGSLRLSCAASGFTLDYYAIGWFRQAPGKEREGVSCISSSGGSTYYADSV
KGRFTISRDNAKNTVYLQMNSLKPEDTAVYYCATACASPVIYEYDYWGQGTQVTVSS,
(SEQ ID NO: 140)
QVQLQESGGGLVQPGGSLRLSCAASGFTLDYYAIGWFRQAPGKEREGVSCISSSGGSTYYADSV
KGRFTISRDNAKNTVYLQMNSLKPEDTAVYYCATDCAGGTSTPYDYWGQGTQVTVSS,
(SEQ ID NO: 141)
QVQLQESGGGLVQPGGSLRLSCAASGFTLGYYAIGWFRQAPGKEREGVSCISSSDGSTYYADSV
KGRFTISRDNAKNTVYLQMNSLKPEDTAVYYCATACADTTQYEYDYWGQGTQVTVSS,
(SEQ ID NO: 142)
QVQLQESGGGLVQPGGSLRLSCAASGFTLGYYAIGWFRQAPGKEREGVSCISSSDGSTYYADSV
KGRFTISRDNAKNTVYLQMNSLKPEDTAVYYCATACANWSSLGPYDYWGQGTQVTVSS,
(SEQ ID NO: 143)
QVQLQESGGGLVQPGGSLRLSCAASGFTLGYYAIGWFRQAPGKEREGVSCISSSDGSTYYADSV
KGRFTISRDNAKNTVYLQMNSLKPEDTGVYYCATACGGATGPYDYWGQGTQVTVSS,
(SEQ ID NO: 144)
QVQLQESGGGLVQPGGSLRLSCEGSGFSLDYYAIGWFRQAPGKEREGVSCISSGDGNTYYADSV
KGRFTISRDNAKNTVYLQMNSLKPEDTAVYYCATDCVGFGSNWFDYWGQGTQVTVSS,
(SEQ ID NO: 145)
QVQLQESGGGLVQPGGSLRLSCTASGFSLDYYAIGWFRQAPGKEREGVACISSRTGSTYYADSV
KGRFTISRDNAKNTVALQMNSLKPEDTAVYYCATACVVVGDQNDYWGQGTQVTVSS,
(SEQ ID NO: 146)
QVQLQESGGGLVQPGGSLRLSCTASGFSLDYYAIGWFRQAPGKEREGVSCISSRTGGTYYADSV
KGRFTISRDDAKNTVYLQMNSLKPEDTAVYYCATACVVVGDRNDYWGQGTQVTVSS,
(SEQ ID NO: 147)
QVQLQESGGGLVQPGGSLRLSCTASGFSLDYYAIGWFRQAPGKEREGVSCISSRTGGTYYADSV
KGRFTISRDNAKNTVYLQMNSLKPEDTAVYYCATACVDTTQYEYDYWGQGTQVTVSS,
(SEQ ID NO: 148)
QVQLQESGGGLVQPGGSLRLSCTASGFSLDYYAIGWFRQAPGKEREGVSCISSRTGGTYYADSV
KGRFTISRDNAKNTVYLQMNSLKPEDTAVYYCATACVVVGDQNDYWGQGTQVTVSS,
(SEQ ID NO: 149)
QVQLQESGGGLVQPGGSLRLSCTASGFSLDYYAIGWFRQAPGKEREGVSCISSRTGNTYYADSV
KGRFTISRDDAKNMVYLQMNSLKPEDTAVYYCATACVVVGDQNDYWGQGTQVTVSS,
(SEQ ID NO: 150)
QVQLQESGGGLVQPGGSLRLSCTASGFSLDYYAIGWFRQAPGKEREGVSCISSRTGSTYYADSV
KGRFTISRDDAKNTVYLQMNSLKPEDTAVYYCATACVVVGDQNDYWGQGTQVTVSS,
(SEQ ID NO: 151)
QVQLQESGGGLVQPGGSLRLSCTASGFSLGYYAIGWFRQALGKEREGVSCISSRTGSTYYADSV
KGRFTVSRDDAKNTVYLQMNSLKPEDTAVYYCATACVVVGDQNDYWGQGTQVTVSS,
(SEQ ID NO: 152)
QVQLQESGGGLVQPGGSLRLSCTASGFSLGYYAIGWFRQAPGKEREGVSCISSRTGSTYYADSV
KGRFAISRDDAKNTVYLQMNSLKPEDTAVYYCATACVVVGDQNDYWGQGTQVTVSS,
(SEQ ID NO: 153)
QVQLQESGGGLVQPGGSLRLSCTASGFSLGYYAIGWFRQAPGKEREGVSCISSRTGSTYYADSV
KGRFTISRDDAKNTVYLQMNSLKPEDTAVYYCATACVVVGDQNDYWGQGTQVTVSS,
(SEQ ID NO: 154)
QVQLQESGGGLVQPGGSLRLSCTASGFSLGYYAIGWFRQAPGKEREGVSCISSRTGSTYYADSV
KGRFTVSRDDAKNTVYLQMNSLKPEDTAVYYCATACVVVGDQNDYWGQGTQVTVSS,
(SEQ ID NO: 155)
QVQLQESGGGLVQPGGSLRLSCVASGFPLDYYAIGWFRQAPGKEREGVSCISSSDGSTYYADSV
KGRFTISRDNAKNTVYLQMNSLKPEDTAVYYCATACGGATGPYDYWGQGTQVTVSS,
(SEQ ID NO: 156)
QVQLQESGGGLVQPGGSLRLSCVASGFSLDYYAIGWFRQAPGKEREGVSCISNSDGSTYYADSV
KGRFTISRDNAKNTVYLQMNSLKPEDTAVYYCATACADTTQYAYDYWGQGTQVTVSS,
(SEQ ID NO: 157)
QVQLQESGGGLVQPGGSLRLSCVASGFTLDYYAIGWFRQAPGKEREGVSCISSGSDGNTYYADS
VKGRFTISRDNAKNTVYLQMNSLKPEDTAVYYCATACSGLTHEYDYWGQGTQVTVSS,
(SEQ ID NO: 158)
QVQLQESGGGLVQPGGSLRLSCVASGFTLDYYAIGWFRQAPGKEREGVSCISSSDDSTYYADSV
KGRFTISRDNAKNTVYLQMNSLKPEDTAVYYCATACADTTQYEYDYWGQGTQVTVSS,
(SEQ ID NO: 159)
QVQLQESGGGLVQPGGSLRLSCVASGFTLDYYAIGWFRQAPGKEREGVSCISSSSDGNTYYADS
VKGRFTISRDNAKNTVYLQMNSLKPEDTAVYYCATTCSGLTHEYDYWGQGTQVTVSS,
(SEQ ID NO: 160)
QVQLQESGGGLVQPGGSLRLSCVASGFTLGYYAIGWFRQAPGKEREGVSCISSSDGSTYYADSV
KGRFTISRDNAKNTVYLQMNSLKPEDTAVYYCATACADTTQYDYDYWGQGTQVTVSS,
(SEQ ID NO: 161)
QVQLQESGGGLVQPGGSLRLSCVGSGFTLDYYAIGWFRQAPGKEREGVSCISSNDGSTYYADSV
KGRFTISRDNAKNTVYLQMNSLKPEDTAVYYCATACGGATGPYDYWGQGTQVTVSS,
(SEQ ID NO: 162)
QVQLQESGGGLVQSGGSLRLSCAASGFPLAYYAIGWFRQAPGKEREGVSCISASDGSTYYADSV
KGRFTISRDNAKNTVYLQMNSLKPEDTAVYYCATACAETTLYEYDYWGQGTQVTVSS,
(SEQ ID NO: 163)
QVQLQESGGGLVQTGGSLRLSCAASGFTLDYYAIGWFRQAPGKEREGVSCISSSDGSTYYADSV
KGRFTISRDNAKNTVYLQMNSLKPEDTAVYYCATACGGATGPYDYWGQGTQVTVSS,
(SEQ ID NO: 164)
QVQLQESGGGMVQAGESLRLSCAASGFPLAYYAIGWFRQAPGKEREGVSCISSSDGNTYYADS
VKGRFTISRDNAKNTVYLQMNSLKPEDTAVYYCATACADATQHEYDYWGQGTQVTVSS,
(SEQ ID NO: 165)
QVQLQESGGGSVQPGESLRLSCAASGFPLDYYAIGWFRQAPGKEREGVSCISASDGSTYYADSV
KGRFTISRDNAKNTVYLQMNSLKPEDTAVYYCATACADTTLYEYDYWGQGTQVTVSS,
(SEQ ID NO: 166)
QVQLQESGGGSVQPGGSLRLSCAASGFTLDYYAIGWFRQAPGKEREGVSCISSGDGSTYYADSV
KGRFTISRDNAKNTVYLQMNSLKPEDTAVYYCATACADTTHYEYDYWGQGTQVTVSS,
(SEQ ID NO: 167)
QVQLQESGGGSVQSGGSLRLSCTASGFSLGYYAIGWFRQAPGKEREGVSCISSRTGSTYYADSV
KGRFTVSRDDAKNTVYLQMNSLKPEDTAVYYCATACVVVGDQNDYWGQGTQVTVSS,
(SEQ ID NO: 168)
QVQLQESGGGSVRPGGSLRLSCAASGFPLAYYAIGWFRQAPGKEREGVSCISSSDGNTYYADAV
KGRFTISRDNAKNAVYLQMNSLKPEDTAVYYCATACADTTQHEYDYWGQGTQVTVSS,
(SEQ ID NO: 169)
QVQLQESGGGVAQPGGSLRLSCAASGFPLDYYAIGWFRQAPGKEREGVSCISASDGSTYYADSV
KGRFTISRDNAKNTVYLQMNSLKPEDTAVYYCATACADTTLYEYDYWGQGTQVTVSS,
(SEQ ID NO: 170)
QVQLQESGGGVVQAGGSLKLSCAASGSDFRADAMGWYRQAPGKEREPVAIDSITSIYYVDSVE
GRFTISRDNTKNTVYLQMTSLKPEDTAVYYCYARYSGRTYWGRGTQVTVSS,
(SEQ ID NO: 171)
QVQLQESGGGVVQPGGSLRLSCAASGFSLDYYAIGWFRQAPGKEREGVSCISSGDGSTYYADSV
KGRFTISRDNAKNTVYLQMNSLKPEDTAVYYCATHCGGTSWGTSYDYWGQGTQVTVSS,
(SEQ ID NO: 172)
QVQLQESGGGVVQPGGSLRLSCAASGLTFRSVGMGWFRRAPGKEREFVATASPSGVITYYADS
VKGRFTISRDNAKNTVYLEMNSLKPEDTAVYYCAVRIYSGSFDNTLAYDYWGQGTQVTVSS,
(SEQ ID NO: 173)
QVQLQESGGGVVQPGGSLRLSCTASGFSLGYYAIGWFRQAPGKEREGVSCISSRTGSTYYADSV
KGRFTVSRDDAKNTVYLQMNSLKPEDTAVYYCATACVVVGDQNDYWGQGTQVTVSS,
and
(SEQ ID NO: 174)
QVQLQESGGGVVQSGGSLRLSCTASGFSLDYYAIGWFRQAPGKEREGVSCISSRTGSTYYADSV
KGRFTISRDDAKNTVYLQMNSLKPEDTAVYYCATACVVVGDQNDYWGQGTQVTVSS

In some embodiments, the VH is a single domain antibody domain. In some embodiments, the VH is a WM.

In some embodiments, the extracellular domain of a CFP comprises an anti-GPC3 variable heavy chain (VH) domain, having a 70-100% sequence identity to

(SEQ ID NO: 175)
QVQLVQSGAEVKKPGASVKVSCKASGYTFTDYEMHWVRQAPGQGLEWM
GALDPKTGDTAYSQKFKGKATLTADKSTSTAYMELSSLTSEDTAVYYC
TRFYSYTYWGQGTLVTVSS.

In some embodiments, the extracellular domain of a CFP comprises an anti-GPC3 variable light chain (VL) domain, having a 70-100% sequence identity to

(SEQ ID NO: 176)
DVVMTQSPLSLPVTPGEPASISCRSSQSLVHSNRNTYLHWYLQKPGQS
PQLLIYKVSNRFSGVPDRFSGSGSGTDFTLKISRVEAEDVGVYYCSQN
THVPPTFGQGTKLEIK.

In some embodiments, the extracellular domain of a CFP comprises an anti-GPC3 scFv, comprising a sequence that has 70%-100% sequence identity to the sequence,

(SEQ ID NO: 177)
QVQLVQSGAEVKKPGASVKVSCKASGYTFTDYEMHWVRQAPGQGLEWMGA
LDPKTGDTAYSQKFKGKATLTADKSTSTAYMELSSLTSEDTAVYYCTRFY
SYTYWGQGTLVTVSSGGGGSGGGGSGGGGSDVVMTQSPLSLPVTPGEPAS
ISCRSSQSLVHSNRNTYLHWYLQKPGQSPQLLIYKVSNRFSGVPDRFSGS
GSGTDFTLKISRVEAEDVGVYYCSQNTHVPPTFGQGTKLEIK.

In some embodiments, the antigen binding domain comprises a sequence of an antigen binding domain provided herein, such as a sequence of an antigen binding domain in Table 1 provided herein. Table 1 shows exemplary sequences of chimeric fusion protein domains and/or fragments thereof that are meant to be non-limiting for the disclosure. Underlines denote the CDR sequences in sequential order of CDR1, CDR2 and CDR3 for the respective heavy and light chains in accordance to the Kabat numbering system. Some exemplary and non-limiting CFP domains with amino acid sequences are provided below.

TABLE 1
Extracellular antigen binding domains and parts thereof:

| | SEQUENCES (CDR sequences shown |
|---|---|
| Domain/Part | as underlined, CDR1, CDR2 and CDR3 in succession) |
| Anti-CD5 scFv | EIQLVQSGGGLVKPGGSVRISCAASGYTFTNYGMNWVRQAPGKGLEWMG |
| | WINTHTGEPTYADSFKGRFTFSLDDSKNTAYLQINSLRAEDTAVYFCTRRG |
| | YDWYFDVWGQGTTVTVSSGGGGSGGGGSGGGGSDIQMTQSPSSLSASVGD |
| | RVTITCRASQDINSYLSWFQQKPGKAPKTLIYRANRLESGVPSRFSGSGSGT |
| | DYTLTISSLQYEDFGIYYCQQYDESPWTFGGGTKLEIK (SEQ ID NO: 178) |
| Anti-CD5 scFv | MWLQSLLLLGTVACSISEIQLVQSGGGLVKPGGSVRISCAASGYTFTNYGM |
| with leader | NWVRQAPGKGLEWMGWINTHTGEPTYADSFKGRFTFSLDDSKNTAYLQI |
| sequence | NSLRAEDTAVYFCTRRGYDWYFDVWGQGTTVTVSSGGGGSGGGGSGGG |
| | GSDIQMTQSPSSLSASVGDRVTITCRASQDINSYLSWFQQKPGKAPKTLIYR |
| | ANRLESGVPSRFSGSGSGTDYTLTISSLQYEDFGIYYCQQYDESPWTFGGGT |
| | KLEIK (SEQ ID NO: 179) |
| Anti-CD5 heavy | EIQLVQSGGGLVKPGGSVRISCAASGYTFTNYGMNWVRQAPGKGLEWMG |
| chain variable | WINTHTGEPTYADSFKGRFTFSLDDSKNTAYLQINSLRAEDTAVYFCTRRG |
| domain | YDWYFDVWGQGTTVTV (SEQ ID NO: 180) |
| Anti-CD5 light | EIQLVQSGGGLVKPGGSVRISCAASGYTFTNYGMNWVRQAPGKGLEWMG |
| chain variable | WINTHTGEPTYADSFKGRFTFSLDDSKNTAYLQINSLRAEDTAVYFCTRRG |
| domain | YDWYFDVWGQGTTVTVSS (SEQ ID NO: 181) |
| Anti-CD5 light | DIQMTQSPSSLSASVGDRVTITCRASQDINSYLSWFQQKPGKAPKTLIYRAN |
| chain variable | RLESGVPSRFSGSGSGTDYTLTISSLQYEDFGIYYCQQYDESPWTFGGGTKL |
| domain | EIK (SEQ ID NO: 182) |
| Anti-HER2 light | DIQMTQSPSSLSASVGDRVTITCRASQDVNTAVAWYQQKPGKAPKLLIYSA |
| chain variable | SFLYSGVPSRFSGSRSGTDFTLTISSLQPEDFATYYCQQHYTTPPTFGQGTK |
| domain | VEIKRTGSTSGSGKPGSGEGSEVQLVE (SEQ ID NO: 183) |
| Anti-HER2 light | DIQMTQSPSSLSASVGDRVTITCRASQDVNTAVAWYQQKPGKAPKLLIYSA |
| chain variable | SFLYSGVPSRFSGSRSGTDFTLTISSLQPEDFATYYCQQHYTTPPTFGQGTK |
| domain | VEIK (SEQ ID NO: 184) |
| Anti-HER2 | LVQPGGSLRLSCAASGFNIKDTYIHWVRQAPGKGLEWVARIYPTNGYTRY |
| heavy chain | ADSVKGRFTISADTSKNTAYLQMNSLRAEDTAVYYCSRWGGDGFYAMDV |
| variable domain | WGQGTLVTV (SEQ ID NO: 185) |
| Anti-HER2 | EVOLVESGGGLVQPGGSLRLSCAASGFNIKDTYIHWVRQAPGKGLEWVAR |
| heavy chain | IYPTNGYTRYADSVKGRFTISADTSKNTAYLQMNSLRAEDTAVYYCSRWG |
| variable domain | GDGFYAMDVWGQGTLVTVSS (SEQ ID NO: 186) |
| Anti-HER2 scFv | DIQMTQSPSSLSASVGDRVTITCRASQDVNTAVAWYQQKPGKAPKLLIYSA |
| | SFLYSGVPSRFSGSRSGTDFTLTISSLQPEDFATYYCQQHYTTPPTFGQGTK |
| | VEIKRTGSTSGSGKPGSGEGSEVQLVESSGGGGSGGGGSGGGGSLVQPGGS |
| | LRLSCAASGFNIKDTYIHWVRQAPGKGLEWVARIYPTNGYTRYADSVKGR |
| | FTISADTSKNTAYLQMNSLRAEDTAVYYCSRWGGDGFYAMDVWGQGTL |
| | VTV (SEQ ID NO: 187) |
| Anti-HER2 scFv | DIQMTQSPSSLSASVGDRVTITCRASQDVNTAVAWYQQKPGKAPKLLIYSA |
| | SFLYSGVPSRFSGSRSGTDFTLTISSLQPEDFATYYCQQHYTTPPTFGQGTK |
| | VEIKRTGSTSGSGKPGSGEGSEVQLVESGGGLVQPGGSLRLSCAASGFNIKD |
| | TYIHWVRQAPGKGLEWVARIYPTNGYTRYADSVKGRFTISADTSKNTAYL |
| | QMNSLRAEDTAVYYCSRWGGDGFYAMDVWGQGTLVTVSS (SEQ ID NO: 188) |
| Anti-HER2 scFv | DIQMTQSPSSLSASVGDRVTITCRASQDVNTAVAWYQQKPGKAPKLLIYSA |
| | SFLYSGVPSRFSGSRSGTDFTLTISSLQPEDFATYYCQQHYTTPPTFGQGTK |
| | VEIKRTGSTSGSGKPGSGEGSEVQLVESGGGLVQPGGSLRLSCAASGFNIKD |
| | TYIHWVRQAPGKGLEWVARIYPTNGYTRYADSVKGRFTISADTSKNTAYL |
| | QMNSLRAEDTAVYYCSRWGGDGFYAMDVWGQGTLVTVSS (SEQ ID NO: 188) |
| Anti-Trop-2 | DIQLTQSPSSLSASVGDRVSITCKASQDVSIAVAWYQQKPGKAPKLLIYSAS |
| binding domain | YRYTGVPDRFSGSGSGTDFTLTISSLQPEDFAVYYCQQHYITPLTFGAGTKV |
| (scFv) | EIKRGGGGSGGGGSGGGGSQVQLQQSGSELKKPGASVKVSCKASGYTFTN |
| | YGMNWVKQAPGQGLKWMGWINTYTGEPTYTDDFKGRFAFSLDTSVSTA |
| | YLQISSLKADDTAVYFCARGGFGSSYWYFDVWGQGSLVTVSS (SEQ ID NO: 189) |
| Anti-Trop-2 | QVQLQQSGSELKKPGASVKVSCKASGYTFTNYGMNWVKQAPGQGLKWM |
| binding domain | GWINTYTGEPTYTDDFKGRFAFSLDTSVSTAYLQISSLKADDTAVYFCARG |
| (scFv) | GFGSSYWYFDVWGQGSLVTVSSGGGGSGGGGSGGGGSDIQLTQSPSSLSA |
| | SVGDRVSITCKASQDVSIAVAWYQQKPGKAPKLLIYSASYRYTGVPDRFSG |
| | SGSGTDFTLTISSLQPEDFAVYYCQQHYITPLTFGAGTKVEIKR (SEQ ID NO: 190) |
| Anti-Trop-2 VH | QVQLQQSGSELKKPGASVKVSCKASGYTFTNYGMNWVKQAPGQGLKWM |
| domain | GWINTYTGEPTYTDDFKGRFAFSLDTSVSTAYLQISSLKADDTAVYFCARG |
| | GFGSSYWYFDVWGQGSLVTVSS (SEQ ID NO: 191) |
| Anti-TROP2 VL | DIQLTQSPSSLSASVGDRVSITCKASQDVSIAVAWYQQKPGKAPKLLIYSAS |
| domain | YRYTGVPDRFSGSGSGTDFTLTISSLQPEDFAVYYCQQHYITPLTFGAGTKV |
| | EIKR (SEQ ID NO: 192) |
| Anti-GPC3 | QVQLVQSGAEVKKPGASVKVSCKASGYTFTDYEMHWVRQAPGQGLEWM |
| binding domain | GALDPKTGDTAYSQKFKGKATLTADKSTSTAYMELSSLTSEDTAVYYCTR |
| variable heavy | FYSYTYWGQGTLVTVSS (SEQ ID NO: 175) |
| chain | |
| Anti-GPC3 | DVVMTQSPLSLPVTPGEPASISCRSSQSLVHSNRNTYLHWYLQKPGQSPQL |
| binding domain | LIYKVSNRFSGVPDRFSGSGSGTDFTLKISRVEAEDVGVYYCSQNTHVPPTF |
| variable light | GQGTKLEIK (SEQ ID NO: 176) |
| chain | |
| Anti-GPC3 | QVQLVQSGAEVKKPGASVKVSCKASGYTFTDYEMHWVRQAPGQGLEWM |
| binding domain | GALDPKTGDTAYSQKFKGKATLTADKSTSTAYMELSSLTSEDTAVYYCTR |
| (scFv) | FYSYTYWGQGTLVTVSSGGGGSGGGGSGGGGSDVVMTQSPLSLPVTPGEP |
| | ASISCRSSQSLVHSNRNTYLHWYLQKPGQSPQLLIYKVSNRFSGVPDRESGS |
| | GSGTDFTLKISRVEAEDVGVYYCSQNTHVPPTFGQGTKLEIK (SEQ ID NO: 177) |
| Anti-CD137 | MEFGLSWLFLVAILKGVQCGLLDLRQGMFAQLVAQNVLLIDGPLSWYSDP |
| binding domain | GLAGVSLTGGLSYKEDTKELVVAKAGVYYVFFQLELRRVVAGEGSGSVSL |
| | ALHLQPLRSAAGAAALALTVDLPPASSEARNSAFGFQGRLLHLSAGQRLG |
| | VHLHTEARARHAWQLTQGATVLGLFRVTPEIPAGLPSPRSE (SEQ ID NO: 193) |
| Anti-CD70 | QVQLVQSGAEVKKPGASVKVSCKASGYTFTNYGMNWVRQAPGQGLEWM |
| binding domain | GWINTYTGEPTYADAFKGRVTMTTDTSTSTAYMELRSLRSDDTAVYYCAR |
| | DYGDYGMDYWGQGTTVTVSSGSTSGSGKPGSSEGSTKGDIVMTQSPDSLA |
| | VSLGERATINCRASKSVSTSGYSFMHWYQQKPGQPPKLLIYLASNLESGVP |
| | DRFSGSGSGTDFTLTISSLQAEDVAVYYCQHSREVPWTFGQGTKVEIK |
| | (SEQ ID NO: 194) |
| Anti- | QVQLQESGPGLVKPSQTLSLTCTVSGGSISSGYNWHWIRQPPGKGLEWIGY |
| Claudin18.2 | IHYTGSTNYNPALRSRVTISVDTSKNQFSLKLSSVTAADTAVYYCARIYNG |
| binding domain | NSFPYWGQGTTVTVSSGGGGSGGGGSGGGGSDIVMTQSPDSLAYSLGERA |
| (scFv) | TINCKSSQSLFNSGNQKNYLTWYQQKPGQPPKLLIYWASTRESGVPDRFSG |
| | SGSGTDIFITISSLQAEDVAVYYCQNAYSFPYTFGGGTKLEIKR (SEQ ID NO: 195) |

TABLE 2
Transmembrane domains and parts thereof:

| Domain/Part | SEQUENCES |
|---|---|
| CD89 | LIRMAVAGLVLVALLAILV |
| | (SEQ ID NO: 196) |
| CD89- hinge-TM | DSIHQDYTTQNLIRMAVAGLVLVALLAIL |
| (Italicized) | VENWHSHTALNKEASADVAEPSWSQQMCQ |
| and ICD | PGLTFARTPSVCK |
| | (SEQ ID NO: 197) |
| CD89 TMD with hinge | IHQDYTTQNLIRMAVAGLVLVALLAILV |
| | (SEQ ID NO: 198) |
| CD32 TMD | SSSPMGIIVAVVTGIAVAAIVAA |
| | (SEQ ID NO: 199) |
| Human CD16 TMD | SFCLVMVLLFAVDTGLYFSV |
| | (SEQ ID NO: 200) |
| Human CD16 TMD | GLAVSTISSFFPPGYQVSFCLVMVLLFAV |
| with hinge | DTGLYFSV |
| | (SEQ ID NO: 201) |
| CD8a transmembrane | IYIWAPLAGTCGVLLLSLVIT |
| domain | (SEQ ID NO: 202) |
| CD8a transmembrane | IYIWAPLAGTCGVLLLSLVITLYC |
| domain | (SEQ ID NO: 203) |
| CD2 Transmembrane | IYLIIGICGGGSLLMVFVALLVFYIT |
| domain | (SEQ ID NO: 204) |
| CD28 transmembrane | FWVLVVVGGVLACYSLLVTVAFIIFWV |
| domain | (SEQ ID NO: 205) |
| CD68 transmembrane | ILLPLIIGLILLGLLALVLIAFCII |
| domain | (SEQ ID NO: 206) |
| CD8α chain | ALSNSIMYFSHFVPVFLPAKPTTTPAPRP |
| hinge domain + | PTPAPTIASQPLSLRPEACRPAAGGAVHT |
| transmembrane domain | RGLDIYIWAPLAGTCGVLLLSLVITLYC |
| | (SEQ ID NO: 207) |
| CD8α chain | ALSNSIMYFSHFVPVFLPAKPTTTPAPRP |
| hinge domain + | PTPAPTIASQPLSLRPEACRPAAGGAVHT |
| transmembrane domain | RGLDIYIWAPLAGTCGVLLLSLVIT |
| | (SEQ ID NO: 208) |

TABLE 3
Intracellular signaling
domains and parts thereof:

| Domain/Part | SEQUENCES |
|---|---|
| FcRγ-chain intracellular | LYCRRLKIQVRKAAITSYEKSDGV |
| signaling domain | YTGLSTRNQETYETLKHEKPPQ |
| | (SEQ ID NO: 209) |
| FcRγ-chain intracellular | LYCRLKIQVRKAAITSYEKSDGVY |
| signaling domain | TGLSTRNQETYETLKHEKPPQ |
| | (SEQ ID NO: 210) |
| FcRγ-chain intracellular | RLKIQVRKAAITSYEKSDGVYTG |
| signaling domain | LSTRNQETYETLKHEKPPQ |
| | (SEQ ID NO: 211) |
| FcRγ-chain intracellular | RLKIQVRKAAITSYEKSDGVYTGL |
| signaling domain | STRNQETYETLKHEKPPQ |
| | (SEQ ID NO: 211) |
| Human CD89 cytoplasmic | ENWHSHTALNKEASADVAEPSWSQ |
| domains | QMCQPGLTFARTPSVCK |
| | (SEQ ID NO: 212) |
| Human CD16 cytoplasmic | KTNIRSSTRDWKDHKFKWRKDPQD |
| domain | K |
| | (SEQ ID NO: 213) |
| PI3K recruitment domain | YEDMRGILYAAPQLRSIRGQPGPN |
| | HEEDADSYENM |
| | (SEQ ID NO: 214) |
| CD40 intracellular | KKVAKKPTNKAPHPKQEPQEINFP |
| domain | DDLPGSNTAAPVQETLHGCQPVTQ |
| | EDGKESRISVQERQ |
| | (SEQ ID NO: 215) |
| TNFR1 intracellular | QRWKSKLYSIVCGKSTPEKEGELE |
| domain | GTTTKPLAPNPSFSPTPGFTPTLG |
| | FSPVPSSTFTSSSTYTPGDCPNFA |
| | APRREVAPPYQGADPILATALASD |
| | PIPNPLQKWEDSAHKPQSLDTDDP |
| | ATLYAVVENVPPLRWKEFVRRLGL |
| | SDHEIDRLELQNGRCLREAQYSML |
| | ATWRRRTPRREATLELLGRVLRDM |
| | DLLGCLEDIEEALCGPAALPPAPS |
| | LLR |
| | (SEQ ID NO: 216) |
| TNFR2 intracellular | PLCLQREAKVPHLPADKARGTQGP |
| domain | EQQHLLITAPSSSSSSLESSASAL |
| | DRRAPTRNQPQAPGVEASGAGEAR |
| | ASTGSSDSSPGGHGTQVNVTCIVN |
| | VCSSSDHSSQCSSQASSTMGDTDS |
| | SPSESPKDEQVPFSKEECAFRSQL |
| | ETPETLLGSTEEKPLPLGVPDAGM |
| | KPS |
| | (SEQ ID NO: 217) |
| MDA5 intracellular | MSNGYSTDENFRYLISCFRARVKM |
| domain | YIQVEPVLDYLTFLPAEVKEQIQR |
| | TVATSGNMQAVELLLSTLEKGVWH |
| | LGWTREFVEALRRTGSPLAARYMN |
| | PELTDLPSPSFENAHDEYLQLLNL |
| | LQPTLVDKLLVRDVLDKCMEEELL |
| | TIEDRNRIAAAENNGNESGVRELL |
| | KRIVQKENWFSAFLNVLRQTGNNE |
| | LVQELTGSDCSESNAEIEN |
| | (SEQ ID NO: 218) |

One of ordinary skill in the art is able to easily construct or reconstruct a variety of combinations of domains into a complete chimeric protein as encompassed by the disclosure. Disclosure of the amino acid sequence also constitute disclosure of the coding sequence other than minor optimizations.

In some embodiments, the chimeric construct encompasses an extracellular antigen binding domain that is at least 80% identical to any one of the sequences presented herein. In some embodiments, the extracellular antigen binding domain that is at least 85% identical to any one of the sequences presented herein. In some embodiments, the extracellular antigen binding domain that is at least 90% identical to any one of the sequences presented herein. In some embodiments, the extracellular antigen binding domain that is at least 91%, 92%, 93%, 94%, 95%, 96%, 97%, 98%, 99%, or about 100% identical to any one of the sequences presented herein.

In some embodiments, a GMCSF signal peptide sequence is incorporated at the N-terminal end. An exemplary GMCSF signal sequence peptide may be MWLQSLLLLGTVACSIS. In some embodiments, one or more linker sequences may be introduced at the junction of two domains, e.g., the extracellular antigen binding domain and the hinge domain, or between the hinge domain and the transmembrane domain or between the transmembrane domain and the intracellular domain, or between two adjacent intracellular domains, for flexibility of the CFP molecule where necessary. In some embodiments, a linker is used to separate two or more polypeptides, e.g., two antigenic peptides by a distance sufficient to ensure that each antigenic peptide properly folds. Exemplary peptide linker sequences adopt a flexible extended conformation and do not exhibit a propensity for developing an ordered secondary structure. Amino acids in flexible linker protein region may include Gly, Asn and Ser, or any permutation of amino acid sequences containing Gly, Asn and Ser. Other near neutral amino acids, such as Thr and Ala, also can be used in the linker sequence.

In some embodiments, the linker is a polypeptide. In some embodiments, the linker is a functional peptide. In some embodiments, the linker is a ligand for a receptor. In some embodiments, the ligand for a monocyte or macrophage receptor. In some embodiments, the linker activates the receptor. In some embodiments, the linker inhibits the receptor. Exemplary structural linkers that are generally lacking particular biochemical neutral in function to incorporate between domains are SGGG (SEQ ID NO: 220); GSGS (SEQ ID NO: 221); SGSGSG (SEQ ID NO: 222); SGGGGSG (SEQ ID NO: 223); SSSGGGGSG (SEQ ID NO: 224); GGGGSGGGGSGGGGS (SEQ ID NO: 225); GGGGSGGGGSGGGG (SEQ ID NO: 226). A chimeric receptor may include one, two, three, four, or five or more linkers. In some embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids long.

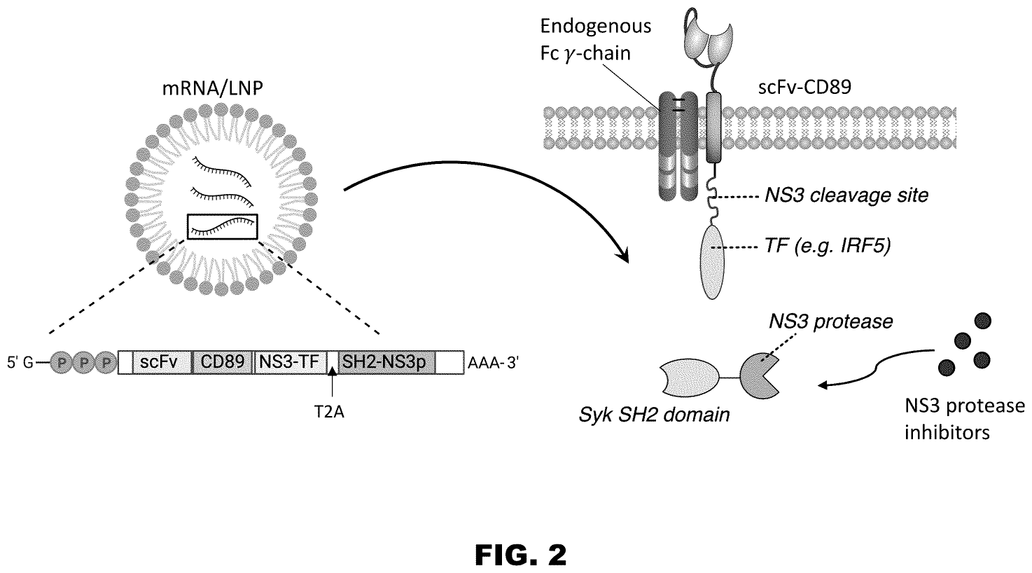

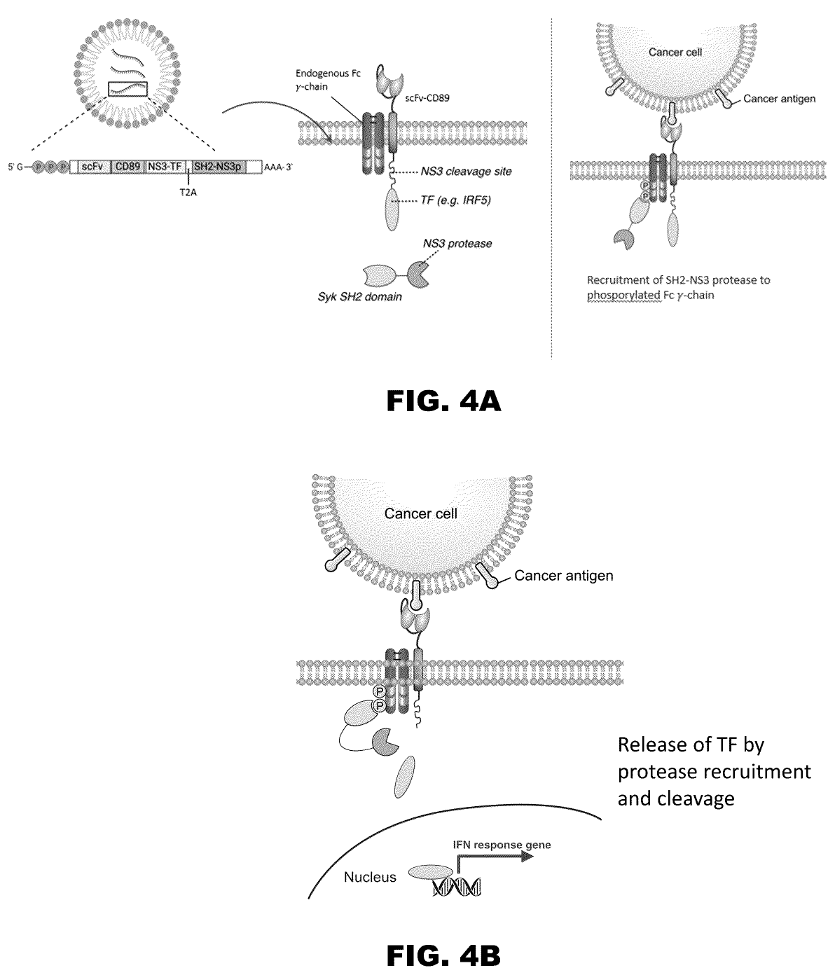

Although specific combinations of domains are disclosed in the tables and paragraphs above, one of skill in the art could choose to incorporate in a chimeric receptor backbone any one or more of the domains disclosed herein, which is fully contemplated in the instant disclosure. A design for the chimeric proteins described herein are schematically illustrated in the figures provided herein, FIG. 1, FIG. 2, FIG. 3 and the mechanism of action explained in FIGS. 4A and 4B. In some embodiments, the intracellular domain comprises a sequence encoding a transcription factor or a functional part thereof, such as a transcriptionally active fraction of a transcription factor. A transcription factor may further encompass a sequence encoding an enhancer. In some embodiments, the transcription factor activates an inflammatory gene transcription. In some embodiments, an exemplary inflammatory gene is an interferon responsive gene. In some embodiments, the transcription factor or the transcriptionally active part thereof is selected from a group of transcription factors or a portions thereof, consisting of NF-kB, STAT1, STATS, IRF1, IRF2, IRF3, IRF4, IRF5, IRF6, IRF7, IRF8 and IRF9. In some embodiments, the transcription factors is IRF5.

Any other cell surface receptor backbone may be utilized as contemplated herein. Such receptors include but are not limited to immune cell receptors, growth factor receptors, cytokine receptors, hormone receptors, receptor tyrosine kinases, immune receptors such as CD28, CD80, ICOS, CTLA4, PD1, PD-L1, BTLA, HVEM, CD27, 4-1BB, 4-1BBL, OX40, OX40L, DR3, GITR, CD30, SLAM, CD2, 2B4, TIM1, TIM2, TIM3, TIGIT, CD226, CD160, LAGS, LAIR1, B7-15 1, B7-H1, and B7-H3, a type I cytokine receptor such as Interleukin-1 receptor, Interleukin-2 receptor, Interleukin-3 receptor, Interleukin-4 receptor, Interleukin-5 receptor, Interleukin-6 receptor, Interleukin-7 receptor, Interleukin-9 receptor, Interleukin-11 receptor, Interleukin-12 receptor, Interleukin-13 receptor, I nterleukin-15 receptor, Interleukin-18 receptor, Interleukin-21 receptor, Interleukin-23 receptor, Interleukin-27 receptor, Erythropoietin receptor, GM-CSF receptor, G-CSF receptor, Growth hormone receptor, Prolactin receptor, Leptin receptor, Oncostatin M receptor, Leukemia inhibitory factor, a type II cytokine receptor such as interferon alpha/beta receptor, interferon-gamma receptor, Interferon type III receptor, Interleukin-10 receptor, Interleukin-20 receptor, Interleukin-22 receptor, Interleukin-28 receptor, a receptor in the tumor necrosis factor receptor superfamily such as Tumor necrosis factor receptor 2 (1B), Tumor necrosis factor receptor 1, Lymphotoxin beta receptor, OX40, CD40, Fas receptor, Decoy receptor 3, CD27, CD30, 4-1BB, Decoy receptor 2, Decoy receptor 1, Death receptor 5, Death receptor 4, RANK, Osteoprotegerin, TWEAK receptor, TACT, BAFF receptor, Herpesvirus entry mediator, Nerve growth factor receptor, B-cell maturation antigen, Glucocorticoid-induced TNFR-related, TROY, Death receptor 6, Death receptor 3, Ectodysplasin A2 receptor, a chemokine receptor such as CCR1, CCR2, CCR3, CCR4, CCR5, CCR6, CCR7, CCR8, CCR9, CCR10, CXCR1, CXCR2, CXCR3, CXCR4, CXCR5, CXCR6, CX3CR1, XCR1, ACKR1, ACKR2, ACKR3, ACKR4, CCRL2, a receptor in the epidermal growth factor receptor (EGFR) family, a receptor in the fibroblast growth factor receptor (FGFR) family, a receptor in the vascular endothelial growth factor receptor (VEGFR) family, a receptor in the rearranged during transfection (RET) receptor family, a receptor in the Eph receptor family, a receptor that can induce cell differentiation (e.g., a Notch receptor), a cell adhesion molecule (CAM), an adhesion receptor such as integrin receptor, cadherin, and selectin to name a few.

Provided herein are methods and compositions to generate therapeutic myeloid cells, both in vitro and in vivo, such as that are engineered to target and kill a diseased cell, such as a cancer cell. Although the disclosure abundantly exemplifies compositions and methods for killing cancer or tumor cell, one of skill in the art would be able to design such macrophages to target another diseased cell, such as an infected cell, without undue experimentation.

In some embodiments, a macrophage is isolated from a subject in need thereof, engineered to express a protein or more than one protein of interest, and is then administered into the subject. In some embodiments, the subject has a cancer or a tumor. The macrophage that is isolated from the subject, is engineered to express a protein or more than one protein of interest, and is then administered into the subject is a therapeutic macrophage that can target and kill a cancer cell or a tumor cell of the subject.

### Second Fusion Protein: Regulator Protease

In one aspect, in the chimeric recombinant receptor designs disclosed herein use of a regulator is demonstrated. In some embodiments, the regulator is a protease. In some embodiments, the protease may be in a cis configuration with respect to and as a part of the receptor molecule itself. In some embodiments, the protease may be expressed in a trans configuration in the cell as a molecule separate from the cell surface receptor as shown in the construct in FIG. 1A (left), wherein the sequence encoding the chimeric receptor is separated from the fusion protein comprising the protease by a post-translational auto-cleavable sequence (e.g., a T2A sequence).

In some embodiments, the protease contemplated herein is a sequence-specific non-human protease for which pharmacological inhibitors are available. In some embodiments, the protease is a viral protease. Non-limiting example viral proteases that may be used with the systems, compositions, and methods provided herein include a hepatitis C virus (HCV) protease, a rhinovirus protease, a coxsackie virus protease, a dengue virus protease, and a tev protease. In some embodiments, the viral protease may be a HCV protease. In certain embodiments, the viral protease is derived from HCV nonstructural protein 3 (NS3). NS3 consists of an N-terminal serine protease domain and a C-terminal helicase domain. By “derived from HCV NS3” is meant the protease is the serine protease domain of HCV NS3 or a proteolytically active variant thereof capable of cleaving a cleavage site for the serine protease domain of HCV NS3. The protease domain of NS3 forms a heterodimer with the HCV nonstructural protein 4A (NS4A), which activates proteolytic activity. A protease derived from HCV NS3 may include the entire NS3 protein or a proteolytically active fragment thereof, and may further include a cofactor polypeptide, such as a cofactor polypeptide derived from HCV nonstructural protein 4A (NS4A), e.g., an activating NS4A region. In some embodiments, the protease is a soluble cytosolic protease. In some embodiments, the protease is fused to a phosphotyrosine binding domain (PTB), forming the second fusion protein, post-translationally or co-translationally detached form the first fusion protein, the chimeric fusion protein which is a receptor protein. In some embodiments, the second fusion protein is intracellularly tethered to the cell membrane via a transmembrane domain or dimerization domain that that dimerizes with Fc-gamma receptor. In some embodiments, the endogenous cell surface receptor, e.g., the Fc-gamma receptor is associated with the chimeric antigen receptor on the membrane but does not comprise the chimeric antigen receptor. In some embodiments, the second fusion protein is intracellularly tethered to the cell membrane via an anchor. In some embodiments, the anchor is the glycosylphosphatidylinositol (GPI) anchor.

In some embodiments, the second fusion protein lacks the viral protease cleavable domain, which is on a separate molecule, such as the chimeric fusion protein, as shown in the schematic drawings of FIG. 1 (right), FIG. 2, FIGS. 4A and 4B.

NS3 protease is highly selective and can be inhibited by a number of non-toxic, cell-permeable drugs, which are currently available for use in humans. In some embodiments, the protease is kept inhibited until the CFP binds to a cancer cell, and is activated, but the use of the protease. In some embodiments, this is achieved by administration of the protease to the subject, externally, that is orally, or systemically. Withdrawal of the protease inhibitor may lead to the activation of the protease.

An amino acid sequence of an exemplary NS3 derived protease may be available in public database (see e.g., NCBI Reference Sequence: NP_803144.1; SEQ ID NO:1 (amino acids 1-193 of NP_803144.1) Table 4 discloses some exemplary protease domain sequences derived from HCV NS3.

TABLE 4
Exemplary NS3 protease domain sequences

| Domain/Part | SEQUENCES |
|---|---|
| HCV-NS3 Protease | APITAYAQQTRGLLGCIITSLTGRDKNQVEGEV |
| sequence 1 | QIVSTATQTFLATCINGVCWAVYHGAGTRTIAS |
| | PKGPVIQMYTNVDQDLVGWPAPQGSRSLTPCTC |
| | GSSDLYLVTRHADVIPVRRRGDSRGSLLSPRPI |
| | SYLKGSSGGPLLCPAGHAVGLFRAAVCTRGVAK |
| | AVDFIPVENLETTMRSPVFTD |
| | (SEQ ID NO: 227) |
| HCV-NS3 Protease | APITAYAQQTRGLLGCIITSLTGRDKNQVEGEV |
| sequence 2 | QIMSTATQTFLATCINGVCWTVYHGAGTRTIAS |
| | PKGPVIQMYTNVDQDLVGWPAPQGSRSLTPCTC |
| | GSSDLYLVTRHADVIPVRRRGDGRGSLLSPRPI |
| | SYLKGSSGGPLLCPAGHAVGLFRAAVCTRGVAK |
| | AVDFIPVENLETTMRSPVFTD |
| | (SEQ ID NO: 228) |
| HCV-NS3 Protease | APITAYAQQTRGLLGCIITSLTGRDKNQVEGEV |
| sequence 3 | QIVSTATQTFLATCINGVCWAVYHGAGTRTIAS |
| | PKGPVIQMYTNVDQDLVGWPAPQGSRSLTPCTC |
| | GSSDLYLVTRHADVIPVRRRGDSRGSLLSPRPI |
| | SYLKGSSGGPLLCPAGHAVGLFRAAVCTRGVAK |
| | AVDFIPVENLETTMRSPVFTD |
| | (SEQ ID NO: 229) |
| HCV-NS3 Protease | APITAYAQQTRGLLGCIITSLTGRDKNQVEGEV |
| sequence 4 | QIVSTATQTFLATCINGVCWTVYHGAGTRTIAS |
| | PKGPVIQMYTNVDQDLVGWPAPQGSRSLTPCTC |
| | GSSDLYLVTRHADVIPVRRRGDSRGSLLSPRPI |
| | SYLKGSSGGPLLCPAGHAVGLFRAAVCTRGVAK |
| | AVDFIPVENLETTMRSPVFTD |
| | (SEQ ID NO: 230) |
| HCV-NS3 Protease | APITAYAQQTRGLLGCIITSLTGRDKNQVEGEV |
| sequence 5 | QIVSTATQTFLATCINGVCWTVYHGAGTRTIAS |
| | PKGPVIQMYTNYDQDLVGWPAPQGSRSLTPCTC |
| | GSSDLYLVTRHADVIPVRRRGDSRGSLLSPRPI |
| | SYLKGSSGGPLLCPAGHAVGLFRAAVCTRGVAK |
| | AVDFIPVENLETTMR |
| | (SEQ ID NO: 231) |

In some embodiments, the protease includes the sequence set forth in the table above, or is a functional (proteolytic) variant thereof having about 70% or greater, 75% or greater, 80% or greater, 85% or greater, 90% or greater, 95% or greater, or 99% or greater amino acid sequence identity to any one of the sequences provided in Table 4, and/or a functional (proteolytic) fragment thereof such as a fragment having a length of from 100 to 185, 120 to 185, 140 to 185, 160 to 185, 170 to 185, from 180 to 185, from 182 to 185, or from 184 to 185 amino acids.

For example, in some embodiments, the regulator protein or the second fusion protein may comprise a protease may comprise a sequence that has at least about 70%, 71%, 72%, 73%, 74%, 75%, 76%, 77%, 78%, 79%, 80%, 81%, 82%, 83%, 84%, 85%, 86%, 87%, 88%, 89%, 90%, 915, 92%, 93%, 94%, 95%, 96%, 97%, 98%, 99%, or 100% sequence identity to an NS3 amino acid sequence: APITAYAQQTRGLLGCIITSLTGRDKNQVEGEVQIVSTATQTFLATCINGVCWAVYHGAGTRTIA SPKGPVIQMYTNVDQDLVGWPAPQGSRSLTPCTCGSSDLYLVTRHADVIPVRRRGDSRGSLLSP RPISYLKGSSGGPLLCPAGHAVGLFRAAVCTRGVAKAVDFIPVENLETTMRSPVFTD (SEQ ID NO: 227). In some embodiments, the second fusion protein may comprise a Syk-SH2 domain, that is fused to the protease via a short linker comprising for example a succession of Glycine and one or more Serine sequences. For example a Syk-SH2 domain may comprise a sequence that has at least about 70%, 71%, 72%, 73%, 74%, 75%, 76%, 77%, 78%, 79%, 80%, 81%, 82%, 83%, 84%, 85%, 86%, 87%, 88%, 89%, 90%, 915, 92%, 93%, 94%, 95%, 96%, 97%, 98%, 99%, or 100% sequence identity to an amino acid sequence:

(SEQ ID NO: 247)
ASSGMADSANHLPFFFGNITREEAEDYLVQGGMSDGLYLLRQSRNYLGGF
ALSVAHGRKAHHYTIERELNGTYAIAGGRTHASPADLCHYHSQESDGLVC
LLKKPFNRPQGVQPKTGPFEDLKENLIREYVKQTWNLQGQALEQAIISQK
PQLEKLIATTAHEKMPWFHGKISREESEQIVLIGSKTNGKFLIRARDNNG
SYALCLLHEGKVLHYRIDKDKTGKLSIPEGKKFDTLWQLVEHYSYKADGL
LRVLTVPC.

For example, in some embodiments, the regulator protein or the second fusion protein may comprise a protease may comprise a sequence that has at least about 70%, 71%, 72%, 73%, 74%, 75%, 76%, 77%, 78%, 79%, 80%, 81%, 82%, 83%, 84%, 85%, 86%, 87%, 88%, 89%, 90%, 915, 92%, 93%, 94%, 95%, 96%, 97%, 98%, 99%, or 100% sequence identity to an NS3 amino acid sequence: APITAYAQQTRGLLGCIITSLTGRDKNQVEGEVQIMSTATQTFLATCINGVCWTVYHGAGTRTIA SPKGPVIQMYTNVDQDLVGWPAPQGSRSLTPCTCGSSDLYLVTRHADVIPVRRRGDGRGSLLSP RPISYLKGSSGGPLLCPAGHAVGLFRAAVCTRGVAKAVDFIPVENLETTMRSPVFTD (SEQ ID NO: 228). In some embodiments, the second fusion protein may comprise a Syk-SH2 domain, that is fused to the protease sequence above via a short linker comprising for example a succession of Glycine and one or more Serine sequences. For example a Syk-SH2 domain may comprise a sequence that has at least about 70%, 71%, 72%, 73%, 74%, 75%, 76%, 77%, 78%, 79%, 80%, 81%, 82%, 83%, 84%, 85%, 86%, 87%, 88%, 89%, 90%, 915, 92%, 93%, 94%, 95%, 96%, 97%, 98%, 99%, or 100% sequence identity to an amino acid sequence:

(SEQ ID NO: 247)
ASSGMADSANHLPFFFGNITREEAEDYLVQGGMSDGLYLLRQSRNYLGGF
ALSVAHGRKAHHYTIERELNGTYAIAGGRTHASPADLCHYHSQESDGLVC
LLKKPFNRPQGVQPKTGPFEDLKENLIREYVKQTWNLQGQALEQAIISQK
PQLEKLIATTAHEKMPWFHGKISREESEQIVLIGSKTNGKFLIRARDNNG
SYALCLLHEGKVLHYRIDKDKTGKLSIPEGKKFDTLWQLVEHYSYKADGL
LRVLTVPC.

For example, in some embodiments, the regulator protein or the second fusion protein may comprise a protease may comprise a sequence that has at least about 70%, 71%, 72%, 73%, 74%, 75%, 76%, 77%, 78%, 79%, 80%, 81%, 82%, 83%, 84%, 85%, 86%, 87%, 88%, 89%, 90%, 915, 92%, 93%, 94%, 95%, 96%, 97%, 98%, 99%, or 100% sequence identity to an NS3 amino acid sequence: APITAYAQQTRGLLGCIITSLTGRDKNQVEGEVQIVSTATQTFLATCINGVCWAVYHGAGTRTIA SPKGPVIQMYTNVDQDLVGWPAPQGSRSLTPCTCGSSDLYLVTRHADVIPVRRRGDSRGSLLSP RPISYLKGSSGGPLLCPAGHAVGLFRAAVCTRGVAKAVDFIPVENLETTMRSPVFTD (SEQ ID NO: 229). In some embodiments, the second fusion protein may comprise a Syk-SH2 domain, that is fused to the protease sequence above via a short linker comprising for example a succession of Glycine and one or more Serine sequences. For example a Syk-SH2 domain may comprise a sequence that has at least about 70%, 71%, 72%, 73%, 74%, 75%, 76%, 77%, 78%, 79%, 80%, 81%, 82%, 83%, 84%, 85%, 86%, 87%, 88%, 89%, 90%, 915, 92%, 93%, 94%, 95%, 96%, 97%, 98%, 99%, or 100% sequence identity to an amino acid sequence:

(SEQ ID NO: 247)
ASSGMADSANHLPFFFGNITREEAEDYLVQGGMSDGLYLLRQSRNYLGGF
ALSVAHGRKAHHYTIERELNGTYAIAGGRTHASPADLCHYHSQESDGLVC
LLKKPFNRPQGVQPKTGPFEDLKENLIREYVKQTWNLQGQALEQAIISQK
PQLEKLIATTAHEKMPWFHGKISREESEQIVLIGSKTNGKFLIRARDNNG
SYALCLLHEGKVLHYRIDKDKTGKLSIPEGKKFDTLWQLVEHYSYKADGL
LRVLTVPC.

For example, in some embodiments, the regulator protein or the second fusion protein may comprise a protease may comprise a sequence that has at least about 70%, 71%, 72%, 73%, 74%, 75%, 76%, 77%, 78%, 79%, 80%, 81%, 82%, 83%, 84%, 85%, 86%, 87%, 88%, 89%, 90%, 915, 92%, 93%, 94%, 95%, 96%, 97%, 98%, 99%, or 100% sequence identity to an NS3 amino acid sequence: APITAYAQQTRGLLGCIITSLTGRDKNQVEGEVQIVSTATQTFLATCINGVCWTVYHGAGTRTIA SPKGPVIQMYTNVDQDLVGWPAPQGSRSLTPCTCGSSDLYLVTRHADVIPVRRRGDSRGSLLSP RPISYLKGSSGGPLLCPAGHAVGLFRAAVCTRGVAKAVDFIPVENLETTMRSPVFTD (SEQ ID NO: 230). In some embodiments, the second fusion protein may comprise a Syk-SH2 domain, that is fused to the protease sequence above via a short linker comprising for example a succession of Glycine and one or more Serine sequences. For example a Syk-SH2 domain may comprise a sequence that has at least about 70%, 71%, 72%, 73%, 74%, 75%, 76%, 77%, 78%, 79%, 80%, 81%, 82%, 83%, 84%, 85%, 86%, 87%, 88%, 89%, 90%, 915, 92%, 93%, 94%, 95%, 96%, 97%, 98%, 99%, or 100% sequence identity to an amino acid sequence:

(SEQ ID NO: 247)
ASSGMADSANHLPFFFGNITREEAEDYLVQGGMSDGLYLLRQSRNYLGGF
ALSVAHGRKAHHYTIERELNGTYAIAGGRTHASPADLCHYHSQESDGLVC
LLKKPFNRPQGVQPKTGPFEDLKENLIREYVKQTWNLQGQALEQAIISQK
PQLEKLIATTAHEKMPWFHGKISREESEQIVLIGSKTNGKFLIRARDNNG
SYALCLLHEGKVLHYRIDKDKTGKLSIPEGKKFDTLWQLVEHYSYKADGL
LRVLTVPC.

For example, in some embodiments, the regulator protein or the second fusion protein may comprise a protease may comprise a sequence that has at least about 70%, 71%, 72%, 73%, 74%, 75%, 76%, 77%, 78%, 79%, 80%, 81%, 82%, 83%, 84%, 85%, 86%, 87%, 88%, 89%, 90%, 915, 92%, 93%, 94%, 95%, 96%, 97%, 98%, 99%, or 100% sequence identity to an NS3 amino acid sequence:

(SEQ ID NO: 231)
APITAYAQQTRGLLGCIITSLTGRDKNQVEGEVQIVSTATQTFLATCING
VCWTVYHGAGTRTIASPKGPVIQMYTNYDQDLVGWPAPQGSRSLTPCTCG
SSDLYLVTRHADVIPVRRRGDSRGSLLSPRPISYLKGSSGGPLLCPAGHA
VGLFRAAVCTRGVAKAVDFIPVENLETTMR.

In some embodiments, the second fusion protein may comprise a Syk-SH2 domain, that is fused to the protease sequence above via a short linker comprising for example a succession of Glycine and one or more Serine sequences. For example a Syk-SH2 domain may comprise a sequence that has at least about 70%, 71%, 72%, 73%, 74%, 75%, 76%, 77%, 78%, 79%, 80%, 81%, 82%, 83%, 84%, 85%, 86%, 87%, 88%, 89%, 90%, 915, 92%, 93%, 94%, 95%, 96%, 97%, 98%, 99%, or 100% sequence identity to an amino acid sequence:

(SEQ ID NO: 247)
ASSGMADSANHLPFFFGNITREEAEDYLVQGGMSDGLYLLRQSRNYLGGF
ALSVAHGRKAHHYTIERELNGTYAIAGGRTHASPADLCHYHSQESDGLVC
LLKKPFNRPQGVQPKTGPFEDLKENLIREYVKQTWNLQGQALEQAIISQK
PQLEKLIATTAHEKMPWFHGKISREESEQIVLIGSKTNGKFLIRARDNNG
SYALCLLHEGKVLHYRIDKDKTGKLSIPEGKKFDTLWQLVEHYSYKADGL
LRVLTVPC.

In some embodiments, as described herein, the intracellular domain further comprises a therapeutic agent. The therapeutic agent may be a transcription factor or a transcriptionally functional portion thereof; that is linked with on one end with a protease cleavable site. In some embodiments, the transcription factor or the transcriptionally active part thereof is selected from a group of transcription factors or a portions thereof, consisting of NF-kB, STAT1, STATS, IRF1, IRF2, IRF3, IRF4, IRF5, IRF6, IRF7, IRF8 and IRF9. The transcription factor is released into the cytoplasm which migrates to the nucleus upon activation of the receptor via engagement of the extracellular antigen binding domain with its ligand or cognate antigen, for example the antigen presented by a cancer cell. Therefore, upon binding to a target cancer cell the chimeric receptor on the myeloid cell is activated, which sends the signal for activation of the intracellular domain, such that the intracellular signaling domain comprising e.g., ITAM domains are phosphorylated at the tyrosine residues. SH2-domain containing signaling proteins are attracted to and bind to the phosphotyrosine residues. In the designs described herein, a SH2-containing domain fused with the protease of interest is translated from the recombinant polynucleic acid, which is thereafter recruited to the membrane proximal intracellular domain of the signaling domains.

In some embodiments, the protease cleavage site is cleaved by a protease upon activation of the CFP in a cell expressing Fc-gamma receptor.

In some embodiments, the CFP undergoes degradation when expressed in a cell that does not express Fc-gamma receptor.

In some embodiments, the SH2-bound protease is attracted to the intracellular domain Fc-gamma receptor which comprises at least one phosphorylated tyrosine residue at the time the chimeric antigen receptor within the dimerization/multimerization complex is engaged to its target ligand on a cancer cell. In some embodiments, the Fcγ-receptor is the only receptor with an intracellular domain comprising a tyrosine residue or an ITAM domain. In some embodiments, the chimeric fusion protein does not comprise an ITAM domain. In such embodiments, the protease in the exemplary SH2-NS3-protease fusion peptide is attracted to the phosphorylated residue on the Fcγ-receptor in the multimerization complex comprising the chimeric fusion protein, and the phosphotyrosine bound SH2-NS3-protease cleaves the cleavable site on the chimeric fusion protein thereby releasing the intracellular TF.

In some embodiments, the protease cleavable site is disposed between the transcription factor and the transmembrane domain of the chimeric fusion protein. In some embodiments, the protease cleavage site is a viral protease cleavage site, that can be acted upon by a protease, wherein the protease is supplied by the encoded recombinant polynucleic acid. As indicated in schematic diagrams of FIGS. 4A and 4B, the protease is expressed via translation from the recombinant polynucleic acid. In some embodiments, the polynucleic acid comprises an auto-cleavable sequence intervening the sequence encoding the chimeric fusion protein with the intracellular transcription factor, and the sequence encoding the protease (or the fusion protein comprising the protease). The protease comprising fusion protein is cleaved post-translationally, leaving the SH2-protease free in the cytoplasm, to be recruited to the phosphotyrosine residues of the intracellular signaling domains, once the receptor is activated, that is, when the receptor binds to the antigen or ligand on a cancer cell via the extracellular antigen binding domain. In absence of any activation of the receptor, the phosphorylation of the tyrosine residues in the intracellular signaling domain does not occur, and therefore the SH2-NS3 protease is not recruited to the intracellular domain of the receptor complex.

In some embodiments, the chimeric fusion protein lacks a protease. In some embodiments the chimeric fusion protein is post-translationally (or co-translationally) cleaved from the protease, and any protein or domain that the protease is fused to, e.g., the phosphotyrosine binding domain, such as the SH2 domain.

In some embodiments, the chimeric fusion protein lacks an intracellular domain that comprises a phosphorylated tyrosine residue. In some embodiments, the intracellular domain of the CFP lacks a phosphorylated tyrosine residue. In some embodiments, the intracellular domain of the CFP lacks a phosphotyrosine binding (PTB) domain.

In some embodiments the polynucleic acid is polycistronic and the SH2-protease fusion protein is encoded by a cistron other than the one encoding the chimeric fusion receptor. In some embodiments, separate mRNAs may be utilized, one encoding the chimeric fusion protein with the receptor and another encoding the fusion protein comprising the protease. For example, when a protease derived from HCV NS3 is employed, the cleavage site should include a NS3 protease cleavage site. An NS3 protease cleavage site may include the four junctions between nonstructural (NS) proteins of the HCV polyprotein normally cleaved by the NS3 protease during HCV infection, including the NS3/NS4A, NS4A/NS4B, NS4B/NS5A, and NS5A/NS5B junction cleavage sites. For a description of NS3 protease and representative sequences of its cleavage sites for various strains of HCV, see, e.g., Hepatitis C Viruses: Genomes and Molecular Biology (S. L. Tan ed., Taylor & Francis, 2006), Chapter 6, pp. 163-206; the disclosure of which is incorporated herein by reference in its entirety. In some embodiments, the protease is derived from HCV NS3 and engineered to include one or more amino acid substitutions relative to the amino acid sequence set forth in the Table above, as HCV NS3 Protease sequence 1. In some embodiments, for example, the protease may include a substitution at the position corresponding to position 54 of the amino acid sequence set forth in HCV-NS3 Protease sequence 1. In some embodiments, such a substitution is a threonine to alanine substitution.

As disclosed herein, the intracellular domain of the CF comprises a therapeutic agent for example a transcription factor, which is separated from the remainder of the chimeric receptor molecule by a regulatable sequence, such as a protease cleavage sequence. In some embodiments, the regulatable sequence is a protease cleavage sequence, wherein the protease cleavage sequence corresponds to the sequence that the protease used in the system (and encoded for in the recombinant polynucleic acid construct) is capable of cleaving. In some embodiments, the protease is derived from HCV NS3 and the cleavage site includes an NS3 protease cleavage site. An NS3 protease cleavage site may include the HCV polyprotein NS3/NS4A, NS4A/NS4B, NS4B/NS5A, and NS5A/NS5B junction cleavage sites or a sequence that is at least 90% identical to the amino acid cleavage site. Representative HCV NS4A/4B protease cleavage sites may include but not limited to DEMEECSQH (SEQ ID NO: 232) and DEMEECSQ1-1L (SEQ ID NO: 233). Representative HCV NS5A/5B protease cleavage sites may include EDVVPCSMG (SEQ ID NO: 234) and EDVVPCSMGS (SEQ ID NO: 235). A representative NS4B/5A protease cleavage site may be ECTTPCSGSWL (SEQ ID NO: 236). In some embodiments, other sequences may be included, for example, SSPPAVTLTH (SEQ ID NO: 237), spacer sequence derived from the NS3 helicase domain. In some embodiments, other sequences may be included, for example, TGCVVIVGRIVLSG (SEQ ID NO: 238), NS4A-derived cofactor strand.

In some embodiments, the protease cleavage site within the first fusion protein has a sequence of: Asp-Glu-Met-Glu-Glu-Cys-Ser-Gln-His-Leu (SEQ ID NO: 233), wherein the protease in the second fusion protein is an HCV NS4A/4B protease.

In some embodiments, the protease cleavage site within the first fusion protein has a sequence of: Glu-Asp-Val-Val-Pro-Cys-Ser-Met-Gly (SEQ ID NO: 234), wherein the protease in the second fusion protein is an HCV NS5A/5B protease.

In some embodiments, the protease comprises at least one mutation. For example, in some embodiments, the HCV NS3 protease comprises any one or more of V36M, T54A, and S122G mutations and/or F43L, Q80K, S122R, and D168Y mutations, In some embodiments, the HCV NS3 protease comprising V36M, T54A, and S122G mutations is resistant to telaprevir (TPV) and sensitive to asunaprevir (ASV) and the HCV NS3 protease comprising F43L, Q80K, S122R, and D168Y mutations is resistant to ASV and sensitive to TPV. Some mutations conferring reduced immunogenicity may be used in the constructs described herein and may include, for example, G15R, 118V, S20N, V55A, Y105A, L106A, H110A, A111G, V113A, V151A, I170V, and V172A mutations (see e.g., Soderholm and Sallberg, J. Infect. Dis., 194(12), 1724-8 (2006); and Soderholm et al., Gut, 55(2), 266-74, (2006), each of which is herein incorporated by reference in its entirety).

In some embodiments, the second fusion protein comprises a degron fusion protein comprising an HCV NS3 protease, wherein addition of an NS3 protease inhibitor can be used to maintain the protease in an inactive form. The degron provides a degradation signal that targets the fusion protein for cellular degradation through either the proteasome or autophagy—lysosome pathway. In some embodiments, the degron is operably linked to the protease such that degradation of the protease prevents cleavage and release of therapeutic agent from the chimeric fusion protein in non-activated conditions, until signal transduction is initiated on a activated chimeric receptor upon receptor-ligand engagement, that is, when in contact with a cancer cell. The degron may be operably linked to the protease, but need not be contiguous with it as long as the degron still functions to direct degradation of the protease. In some embodiments, the degron may help degrade the protease inhibitor. In some embodiments the degron may be activated upon receiving adequate signal, such as HIF1a related degron. In some embodiments, the degradation activity of the degron is inhibited by binding of the PTB domain, e.g., the SH2 fusion domain.

In some embodiments, one or more protease inhibitors may be used to suppress or regulate the protease. NS3 protease inhibitors that may be used include, but are not limited to, simeprevir, danoprevir, asunaprevir, ciluprevir, boceprevir, sovaprevir, paritaprevir, telaprevir, grazoprevir, and any combination thereof. A protease inhibitor further regulates the activity of the regulator, which is, the protease for as long as the protease is not necessary to act. It can be manipulated externally by administering the protease to the subject. The timing of the administering of the protease may be determined prior to the decision regarding the course of the therapy. In one embodiment the protease inhibitor is administered orally or systemically, which when taken up by the cell expressing the recombinant fusion protein(s) would specifically suppress the protease, until the cell migrates to the tumor. In some embodiments, the subject may be administered the protease for a period before administering the composition comprising the recombinant polynucleic acid encoding the chimeric fusion protein as a therapy, and the protease inhibitor administration may be halted at day 0, or at day 1 or at day 2 from the administration of the administering the composition comprising the recombinant polynucleic acid encoding the chimeric fusion protein.

In some embodiments, the protease is fused to a protein binding domain that can bind to a phosphotyrosine residue on another protein. In some embodiments, the phosphotyrosine binding domain is an SHC or an SH2 domain. In some embodiments, the phosphotyrosine binding domain may comprise an amino acid sequence set forth below:

(SEQ ID NO: 239)
MGKPLHPNDKVMGPGVSYLVRYMGCVEVLQSMRALDENTRTQVTREAISL
VCEAVPGAKGATRRRKPCSRPLSSILGRSNLKFAGMPITLTVSTSSLNLM
AADCKQIIANHHMQSISFASGGDPDTAEYVAYVAKDPVNQRACHILECPE
GLAQDVISTIGQAFELRFKQYLRDIEQVPQQPTLK.

In some embodiments, the protease is fused to a phosphotyrosine binding protein domain, (e.g., an SH2 domain) as shown herein, and comprises an additional sequence such as an HIF1a domain. The hypoxia inducible factor 1a (HIF1a) may comprise an additional regulator of the regulator such that the fusion protein is to be activated in a hypoxic environment, such as within a tumor. In some embodiments, the HIF1a domain has an amino acid sequence of: Met-Leu-Ala-Pro-Tyr-Ile-Pro (SEQ ID NO: 240).

In some embodiments, the phosphotyrosine binding domain with a HIF1a domain comprises a sequence of:

(SEQ ID NO: 241)
MGKPLHPNDKVMGPGVSYLVRYMGCVEVLQSMRALDENTRTQVTREAISL
VCEAVPGAKGATRRRKPCSRPLSSILGRSNLKFAGMPITLTVSTSSLNLM
AADCKQIIANHHMQSISFASGMLAPYIPEYVAYVAKDPVNQRACHILECP
EGLAQDVISTIGQAFELRFKQYLRDIEQVPQQPTLK.

### Recombinant Polynucleic Acid, Pharmaceutical Composition and Delivery

Provided herein is a pharmaceutical composition comprising a polynucleic acid, and suitable for delivery into a myeloid cell. In some embodiments, the polynucleic acid is in the form of a messenger RNA, wherein the messenger RNA encodes a protein or a peptide. In some embodiments, the messenger RNA encodes a domain that facilitates preferential expression of the recombinant protein in a myeloid cell. In some embodiments, the delivery vehicle or the recombinant nucleic acid molecule may comprise a targeting moiety that delivers the mRNA-delivery nanoparticle (e.g., LNP) composition to a myeloid cell. The targeting moiety may be a protein or a peptide. The protein or peptide is designed to express in a myeloid cell thereby rendering the myeloid cell therapeutically effective. In some embodiments, naked DNA or messenger RNA (mRNA) may be used. In some embodiments, DNA or mRNA encoding the protein or peptide (e.g., a chimeric antigen receptor) is encapsulated in a lipid nanoparticle (LNP). The pharmaceutical composition comprises a targeting moiety that delivers or targets the polynucleic acid, or the nanoparticle encapsulated polynucleic acid to a myeloid cell. Targeting moieties specific for myeloid cells are describes in the disclosure. Targeting moieties described herein may be conjugated to the nanoparticle or directly to the polynucleic acid. Conjugation may be via chemical crosslinkers, spacers or peptide or aptamer linkers.

Provided herein is a pharmaceutical composition comprising the composition as described above and a pharmaceutically acceptable excipient.

The pharmaceutical composition is formulated for delivery in vivo, e.g., locally or systemically in a sterile injectable formula.

### Nucleic Acid Cargo

In some embodiments, the polynucleic acid is an mRNA. mRNA is single stranded and may be codon optimized. In some embodiments the mRNA may comprise one or more modified or unnatural bases such as 5′-Methylcytosine, or Pseudouridine or methyl pseudouridine. In some embodiments greater than or about 50% uridine (‘U’) residues of the mRNA may be converted to methyl-pseudouridine. In some embodiments, the mRNA may be 50-10,000 bases long. In one aspect the transgene is delivered as an mRNA. The mRNA may comprise greater than about 100, 200, 300, 400, 500, 600, 700, 800, 900, 1000, 1100, 1200, 1300, 1400, 1500, 1600, 1700, 1800, 1900, 2000, 3000, 4000, 5000, 6000, 7000, 8000, 9000, 10,000 bases. In some embodiments, the mRNA may be more than 10,000 bases long. In some embodiments, the mRNA may be about 11,000 bases long. In some embodiments, the mRNA may be about 12,000 bases long. In some embodiments, the mRNA comprises a transgene sequence that encodes a fusion protein.

In some embodiments, the polynucleic acid can be in the form of a circular RNA (circRNAs). In circular RNAs (circRNAs) the 3′ and 5′ ends are covalently linked. CircRNA may be delivered inside a cell using LNPs.

In some embodiments, the myeloid cell may be modified by expressing a transgene that may be temporally regulated by a regulator from outside the cell. Examples include the Tet-on Tet-off system, where the expression of the transgene is regulated via presence or absence of tetracycline.

In some embodiments, the polynucleic acid is a DNA. The DNA encoding a peptide or a protein that can be expressed in a myeloid cell may be encapsulated in a nanoparticle or a liposome.

LNP encapsulated DNA or RNA can be targeted to a myeloid cell by conjugating with a targeting moiety. In some embodiments the targeting moiety is conjugated to the surface of the LNP. In some embodiments, the targeting moiety is conjugated directly to the nucleic acid.

In some embodiments, the myeloid cell is modified, or engineered to express a recombinant protein comprising an antibody or a fragment thereof.

In some embodiments, the recombinant nucleic acid is DNA. In some embodiments, the recombinant nucleic acid is RNA. In some embodiments, the recombinant nucleic acid is mRNA. In some embodiments, the recombinant nucleic acid is an unmodified mRNA. In some embodiments, the recombinant nucleic acid is a modified mRNA. In some embodiments, the recombinant nucleic acid is a circRNA. In some embodiments, the recombinant nucleic acid is a tRNA. In some embodiments, the recombinant nucleic acid is a microRNA.

Also provided herein is a vector comprising a recombinant nucleic acid sequence encoding a CFP described herein. In some embodiments, the vector is viral vector. In some embodiments, the viral vector is a retroviral vector or a lentiviral vector. In some embodiments, the vector further comprises a promoter operably linked to at least one nucleic acid sequence encoding one or more polypeptides. In some embodiments, the vector is polycistronic. In some embodiments, each of the at least one nucleic acid sequence is operably linked to a separate promoter. In some embodiments, the vector further comprises one or more internal ribosome entry sites (IRESs). In some embodiments, the vector further comprises a 5′UTR and/or a 3′UTR flanking the at least one nucleic acid sequence encoding one or more polypeptides. In some embodiments, the vector further comprises one or more regulatory regions. mRNA is single stranded and may be codon optimized. In some embodiments, the mRNA may comprise one or more modified or unnatural bases such as 5′-Methylcytosine, or Pseudouridine or methyl pseudouridine. In some embodiments greater than or about 50% uridine (‘U’) residues of the mRNA may be converted to methyl-pseudouridine. In some embodiments, the mRNA may be 50-10,000 bases long. In one aspect the transgene is delivered as an mRNA. The mRNA may comprise greater than about 100, 200, 300, 400, 500, 600, 700, 800, 900, 1000, 1100, 1200, 1300, 1400, 1500, 1600, 1700, 1800, 1900, 2000, 3000, 4000, 5000, 6000, 7000, 8000, 9000, 10,000 bases. In some embodiments, the mRNA may be more than 10,000 bases long. In some embodiments, the mRNA may be about 11,000 bases long. In some embodiments, the mRNA may be about 12,000 bases long. In some embodiments, the mRNA comprises a transgene sequence that encodes a fusion protein. LNP encapsulated DNA or RNA can be used for transfecting a macrophage or can be administered to a subject. In some embodiments, the mRNA is incorporated into an effector myeloid cell population by transient transfection. In some embodiments the transient transfection method comprises electroporation of the mRNA. In some embodiments, the transient transfection comprises chemical transfection. In some embodiments, 1-5,000 micrograms/ml of the mRNA may be used for transfection using a suitable protocol for the methods described above. In some embodiments, 1-2,000 micrograms/ml of the mRNA may be used for transfection. In some embodiments, 1-1,000 micrograms/ml of the mRNA may be used for transfection. In some embodiments, 1-1,000 micrograms/ml of the mRNA may be used for transfection. In some embodiments, 1-500 micrograms/ml of the mRNA may be used for transfection. In some embodiments, 1-250 micrograms/ml of the mRNA may be used for transfection. In some embodiments, about 500 micrograms/ml of the mRNA or less may be used for transfection. In some embodiments, about 250 micrograms/ml of the mRNA or less may be used for transfection. In some embodiments, about 10 micrograms/ml of the mRNA is used. In some embodiments, about 20 micrograms/ml of the mRNA is used. In some embodiments, about 30 micrograms/ml of the mRNA is used. In some embodiments, about 40 micrograms/ml of the mRNA is used. In some embodiments, about 50 micrograms/ml of the mRNA is used. In some embodiments, about 60 micrograms/ml of the mRNA is used. In some embodiments, about 80 micrograms/ml of the mRNA is used. In some embodiments, about 100 micrograms/ml of the mRNA is used. In some embodiments, about 150 micrograms/ml of the mRNA is used. In some embodiments, about 200 micrograms/ml of the mRNA is used. In some embodiments, 20, 50, 100, 150, 200, 250, 300, 400, 500 or about 1000 micrograms/ml of the mRNA is used. A suitable cell density is selected for a transfection, based on the method and instrument and/or reagent manufacturer's instructions, or as is well-known to one of skill in the art.

In some embodiments the recombinant nucleic acid is an mRNA. mRNA constructs may be thawed on ice and gently pipetted to monocytes and pre-mixed. In some embodiments, the mRNA is electroporated into the cells. Cells following elutriation may be pooled, centrifuged and may be subjected to electroporation with mRNA using MaxCyte ATX system optimized for the said purpose. In some embodiments, optimized electroporation buffer, cell density, and/or mRNA concentration is used for each protocol for each construct.

In some embodiments, a polynucleotide may be introduced into a myeloid cell in the form of a circular RNA (circRNAs). In circular RNAs (circRNAs) the 3′ and 5′ ends are covalently linked. CircRNA may be delivered inside a cell using LNPs.

In some embodiments, the recombinant polynucleic acid is an mRNA. In some embodiments, the recombinant nucleic acid is an mRNA. In some embodiments, the recombinant nucleic acid is associated with one or more lipids. In some embodiments, the recombinant nucleic acid is encapsulated in a liposome. In some embodiments, the liposome is a lipid nanoparticle.

### Delivery Vehicle

In some embodiments, the recombinant polynucleic acid is encapsulated in a liposome. In some embodiments, the liposome is a lipid nanoparticle. In some embodiments, the recombinant nucleic acid is encapsulated in polymeric nanoparticles.

In some embodiments, the recombinant polynucleic acid is encapsulated in a lipid nanoparticle. In some embodiments, the lipid nanoparticle comprises (a) a nucleic acid; (b) a cationic lipid; (c) a non-cationic lipid; and (d) a conjugated lipid that inhibits aggregation of particles. In some embodiments, the nucleic acid comprises a charged polyanionic nucleic acid. A lipid nanoparticle may comprise a polar lipid. In some embodiments, the lipid nanoparticle comprises a cationic lipid. Cationic lipids have a head group with permanent positive charges. In some embodiments, the lipid nanoparticle comprises a cationic lipid and a non-cationic lipid. In some embodiments, the lipid nanoparticle comprises a neutral lipid. In some embodiments, the lipid nanoparticle comprises a PEGylated lipid. In some embodiments, the delivery vehicle encapsulates the recombinant polynucleic acid. A lipid nanoparticle for use in delivery of a nucleic acid, e.g., mRNA as in the present context comprises any one or more of the lipid components: 306Oᵢ₁₀, tetrakis(8-methylnonyl) 3,3′,3″,3′-(((methylazanediyl) bis(propane-3,1 diyl))bis (azanetriyl))tetrapropionate; 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; β-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; C12-200, 1,1′-((2-(4-(2-((2-(bis(2-hydroxydodecyl)amino)ethyl) (2-hydroxydodecyl)amino)ethyl) piperazin-1-yl)ethyl)azanediyl) bis(dodecan-2-ol); cKK-E12, 3,6-bis(4-(bis(2-hydroxydodecyl)amino)butyl) piperazine-2,5-dione; DC-Cholesterol, 3β-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DOTAP, 1,2-dioleoyl-3-trimethylammonium-propane; DOTMA, 1,2-di-O-octadecenyl-3-trimethylammonium-propane; DSPC, 1,2-distearoyl-sn-glycero-3-phosphocholine; ePC, ethylphosphatidylcholine; FTTS, hexa(octan-3-yl) 9,9′,9″,9″,9″″,9″″-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9Z,12Z,12′Z,12″Z,12′″Z)-tetrakis (octadeca-9,12-dienoate); PEG2000-DMG, 1,2-dimyristoyl-rac-glycero-3-methoxypolyethylene glycol-2000; TT3, N¹,N³,N⁵-tris(3-(didodecylamino)propyl)benzene-1,3,5-tricarboxamide. In some embodiments, the lipid nanoparticle comprises any one of the cationic lipid components, DOTMA (1,2-di-O-octadecenyl-3-trimethylammonium-propane), or DOTAP (1,2-dioleoyl-3-trimethylammonium-propane) or DOPE (1,2-dioleoyl-sn-glycero-3-phosphoethanolamine). In some embodiments, the ionizable lipids may be used. Ionizable lipids are protonated at low pH, which renders them positively charged, that promote membrane destabilization and endosomal escape of the nanoparticle. Exemplary nanoparticles are (2S)-2,5-bis(3-aminopropylamino)-N-[2-(dioctadecylamino)acetyl]pentanamide, (DOGS), N¹-[2-((1S)-1-[(3-aminopropyl)amino]-4-[di(3-aminopropyl)amino]butylcarboxamido) ethyl]-3,4-di[oleyloxy]-benzamide (MVL5), DC-Cholesterol, N⁴-cholesteryl-spermine (GL67), 2,2-dilinoleyl-4-dimethylaminoethyl-[1,3]-dioxolane (DLin-KC2-DMA) and DLin-KC2-DMA led to (6Z,9Z,28Z,31Z)-heptatriaconta-6,9,28,31-tetraen-19-yl 4-(dimethylamino) butanoate (DLin-MC3-DMA; MC3). In some embodiments, the lipid nanoparticle comprises (6Z,9Z,28Z,31Z)-heptatriaconta-6,9,28,31-tetraen-19-yl 4-(dimethylamino) butanoate (DLin-MC3-DMA; MC3. In some embodiments, any one or more of the nanoparticle components may be functionalized to attach a targeting moiety.

In some embodiments, the delivery vehicle is an exosome or an extracellular vesicle. In some embodiments, the exosome or extracellular vesicle is electroporated with the recombinant polynucleic acid. In some embodiments, the exosome or extracellular vesicle is obtained from a cell electroporated with the recombinant polynucleic acid.

In one aspect, provided herein is a method of making the pharmaceutical composition comprising a polynucleic acid encoding a protein or peptide, and a targeting agent for delivery to a myeloid cell. In some embodiments, the polynucleic acid is mRNA. The recombinant construct is prepared using molecular cloning techniques, followed by generating mRNA using in vitro transcription. The mRNA is purified using LC, HPLC, or filtration techniques.

The mRNA may be associated with one or more lipid components to form a liposome, or encapsulated in a lipid nanoparticle.

In some embodiments, the polynucleic acid is encapsulated in an endosome. In some embodiments, the method of preparing an endosomal composition as described above, comprises: (a) introducing the recombinant polynucleic acid into a cell; and (b) obtaining exosomes from the cell, wherein the exosomes comprise the recombinant polynucleic acids.

Provided herein is a method of making the composition as described above, comprising combining the delivery vehicle with the targeting agent and/or the recombinant polynucleic acid.

In some embodiments, the therapy comprises administering a pharmaceutical composition that comprise a cell that have been engineered ex vivo to express a recombinant polynucleic acid construct as described herein. In some embodiments, the cell is a CD14+ cell. In some embodiments, the cell is a CD14+ CD16+ cell. In some embodiments, the cell is a CD14+ CD16− cell. In some embodiments, the cell is an isolated cell. In some embodiments, the cell is isolated from a human subject. In some embodiments the cell is isolated from a biological sample from the subject, wherein the biological sample is peripheral blood. In some embodiments the biological sample is a tissue sample, e.g., a dissected tumor tissue sample. In some embodiments the therapeutic cell composition a myeloid cell, such as a CD14+ cell, a CD14+/CD16− cell, a CD14+/CD16+ cell, a CD14−/CD16+ cell, CD14−/CD16− cell, a dendritic cell, an M0 macrophage, an M2 macrophage, an M1 macrophage or a mosaic myeloid cell/macrophage/dendritic cell. In some embodiments, provided herein is a therapeutic composition comprising at least 20%, at least 30%, at least 40% or at least 50% CD14+ cells. In some embodiments, the therapeutic composition comprises at least 20%, at least 30%, at least 40% or at least 50% CD14+/CD16− cells. In some embodiments, provided herein is a therapeutic composition comprising less than 20%, less than 15%, less than 10% or less than 5% dendritic cells. The myeloid cell for the therapeutic composition as described herein, comprises a recombinant nucleic acid that encodes a chimeric fusion protein encoding a CFP receptor protein or an engager protein as described herein.

### Methods for Generation of Novel Chimeric Receptors Fusion Proteins (CFP) Constructs

In one aspect, provided herein is a method for generating novel chimeric receptor proteins, including, for example, identification of novel domains that can be useful in augmenting a myeloid cell function such that when the fusion receptor is expressed in a myeloid cell, it functions as an effector myeloid cell of the specifications described herein. Generation of fusion proteins as described herein can be performed using well known molecular cloning techniques, and the sequences can be verified after generating of the recombinant nucleic acid.

### Preparation of Recombinant Nucleic Acid Encoding a Chimeric Antigen Receptor:

Recombinant nucleic acid constructs are prepared that encode chimeric antigen receptor (CAR) designed for expression in a myeloid cell and are incorporated in plasmid vectors for amplification and/or testing expression in an eukaryotic cell. The recombinant CARs are constructed using molecular cloning techniques known in the art. A recombinant CAR protein comprises an intracellular domain, a transmembrane domain and an extracellular domain. Each domain or subsection of a domain can be encoded by a nucleic acid sequence that is generated by PCR from heterologous source sequences, and pieced together by cloning individually into the vector, or ligated into a longer nucleic acid that is then inserted into the multi-cloning sites of a suitable plasmid or vector with appropriate promoter and 3′-regulatory elements for amplification. Briefly, an exemplary CAR is prepared by incorporating a nucleic sequence encoding one or more signaling domains, (e.g., a PI3Kinase recruiting domain), a nucleic acid sequence encoding the CD8 hinge and transmembrane domain, a nucleic acid sequence encoding an extracellular domain, having a sequence encoding a target antigen binding scFv at the extracellular end. Certain constructs include a FLAG peptide sequence at the extracellular end designed such that it does not pose hindrance to the scFv binding to its target antigen. These components are ligated together into a sequence that encode a fully functional transmembrane CAR. The nucleic acid subunits encoding individual domains of the recombinant protein is designed to include intervening short flexible linker sequences between two domains. The construct is ligated in a plasmid having a promoter and 3′ stabilizing structural units. In one variation, the construct is placed within an Alu retrotransposon element that encodes ORF2p and has the respective 5′- and 3′-UTR sequences, a CMV promoter. The plasmid is amplified in E. coli, validated by sequencing or stored in (−) 80° C.

mRNA Preparation:

mRNA can be prepared by in vitro transcription using the digested plasmid as template and purified to remove contaminant DNA and polyadenylated. The RNA product is purified, resuspended to 1 mg/ml in RNase free water and stored in cryovials.

Identification of useful CFP ECD, TM, ICD and antigen binding domains for the generation of novel CFPs can be done using the method described herein. Briefly, a large number of potential candidate proteins can be screened for enhanced phagocytic properties and their respective phagocytosis related intracellular signaling. The useful domains can be then used for generation of novel CFPs.

The screen can be divided in two parts: A. Screening for the phagocytic receptor (PR) domains; B. Screening for the antigen binding domains.

### Screening for the PR Domains:

In some embodiments, about 5,800 plasma membrane proteins were screened for their phagocytic potential following the general method described herein. J774 macrophage cells can be transiently transfected with the library of 5800 plasma proteins. High-throughput multiplex assays (ranging from 6-well plate assay set up to up to 384-well plate assay with robotic handling) can be set up to evaluate various potential functions of the plasma membranes. Exemplary assays include, but are not limited to phagocytosis assay, cytokine production assay, inflammasome activation assay, and iNOS activation assay. Exemplary simplified methods can be described in the following paragraphs. Variations of each method can be also used and can be understood by a skilled artisan. Variations of each method can be also used and can be understood by a skilled artisan. Exemplary intracellular signaling domains tested for include but are not limited to CD40-FcRγ; FcRγ-CD40; NLRP3; FcRγ-SH2-Procaspase; FcRγ-Myd88; FcRγ-IFN receptor; FcR-TNFR1; FcRγ-TNFR2; FcR-AIM2; FcRγ-TRIFN; FcRγ-Procaspase; TRIFC; RIG1; MDA5; TBK; CD64; CD16A; CD89; FcRε; SIRPβ; (two consecutive intracellular domains can be represented as hyphenated terms, for example, FcRγ-Myd88 refers to an intracellular domain comprising an FcRγ intracellular signaling domain as signaling domain 1; and an Myd88 intracellular signaling domain as signaling domain 2). The extracellular linker domains screened include but are not limited to CD64, CD16A, CD89, SIRPα, FcRε, CD8 hinge. The transmembrane domains tested include but are not limited to CD8, CD64, CD16A, CD89, FcRε, SIRPα, TNFR1 and CD40. MDA5 domains were also screened.

### Phagocytosis Assay:

Antigen-linked silica or polysterene beads ranging in diameters 1 nm, 5 nm or 10 nm were used for a screen of macrophages. Inert beads can be coated in a supported lipid bilayer and the antigens can be ligated to the lipid bilayer. J774 macrophage cell lines can be prepared, each cell line expressing a cloned recombinant plasma membrane protein. The recombinant plasma membrane protein may also express a fluorescent tag. The cell lines can be maintained and propagated in complete RPMI media with heat inactivated serum and antibiotics (Penicillin/Streptomycin). On the day of the assay, cells can be plated at a density of 1×10\hat{ }6 cells/ml per well in 6 well plates or in a relative proportion in 12 or 24 well plates, and incubated for 2-6 hours. The cells can be then washed once in Phosphate Buffer Saline, and the beads can be added in serum depleted or complement depleted nutrient media. Cells can be visualized by light microscopy at 30 minutes and 2 hours after addition of the beads.

Immunofluorescence reaction may be performed using tagged antibody, and fluorescent confocal microscopy is used to detect the interaction and co-localization of cellular proteins at engulfment. Confidence levels can be determined by Kruskal-Wallis test with Dunn's multiple comparison correction.

In some examples, dye loaded tumor cells can be fed to macrophage cell lines and phagocytosis is assessed by microscopy.

### Cytokine Production:

Macrophage cell lines can be cultured as described above. In one assay, each J774 cell line expressing a plasma membrane protein is plated in multi-wells and challenged with antigen-linked beads and cytokine production was assayed by collecting the supernatants at 4 hours and 24 hours. Cytokines can be assayed from the supernatant by ELISA. In another fraction, cells can be collected at 4 and 24 hours after incubation with the beads and flow cytometry is performed for detection of cytokines. In each case, multiple cytokines can be assayed in a multiplex format, which can be selected from: IL-1α, IL-1β, IL-6, IL-12, IL-23, TNF-α, GMCSF, CXCL1, CXCL3, CXCL9, CXCL-10, MIP1-α and MIP-2. Macrophage inflammatory cytokine array kit (R&D Systems) is used.

Intracellular signaling pathway for inflammatory gene and cytokine activation can be identified by western blot analysis for phosphorylation of MAP kinases, JNK, Akt signaling pathway, Interferon activation pathway including phosphorylation and activation of STAT-1.

### Functional Assays

### Inflammasome Activation Assay:

Activation of NLRP3 inflammasome is assayed by ELISA detection of increased IL-1 production and detection caspase-1 activation by western blot, detecting cleavage of procaspase to generate the shorter caspase. In a microwell plate multiplex setting, Caspase-Glo (Promega Corporation) is used for faster readout of Caspase 1 activation.

iNOS Activation Assay:

Activation of the oxidative burst potential can be measured by iNOS activation and NO production using a fluorimetric assay NOS activity assay kit (AbCAM).

### Cancer Cell Killing Assay:

Raji B cells can be used as cancer antigen presenting cells. Raji cells can be incubated with whole cell crude extract of cancer cells, and co-incubated with J774 macrophage cell lines. The macrophages can destroy the cells after 1 hour of infection, which can be detected by microscopy or detected by cell death assay.

### Screening for High Affinity Antigen Binding Domains:

Cancer ligands can be subjected to screening for antibody light chain and heavy chain variable domains to generate extracellular binding domains for the CFPs. Human full length antibodies or scFv libraries can be screened. Also, potential ligands can be used for immunizing llama for development of novel immunoglobulin binding domains in llama, and preparation of single domain antibodies.

Specific useful domains identified from the screens can be then reverse transcribed, and cloned into lentiviral expression vectors to generate the CFP constructs. A recombinant nucleic acid encoding a CFP can generated using one or more domains from the extracellular, TM and cytoplasmic regions of the highly phagocytic receptors generated from the screen. Briefly plasma membrane receptors showing high activators of pro-inflammatory cytokine production and inflammasome activation can be identified. Bioinformatics studies can be performed to identify functional domains including extracellular activation domains, transmembrane domains and intracellular signaling domains, for example, specific kinase activation sites, SH2 recruitment sites. These screened functional domains can be then cloned in modular constructions for generating novel CFPs. These can be candidate CFPs, and each of these chimeric constructs is tested for phagocytic enhancement, production of cytokines and chemokines, and/or tumor cell killing in vitro and/or in vivo. A microparticle based phagocytosis assay was used to examine changes in phagocytosis. Briefly, streptavidin coupled fluorescent polystyrene microparticles (6 μm diameter) can be conjugated with biotinylated recombinantly expressed and purified cancer ligand. Myeloid cells expressing the novel CFP can be incubated with the ligand coated microparticles for 1-4 h and the amount of phagocytosis was analyzed and quantified using flow cytometry. Plasmid or lentiviral constructions of the designer CFPs can be then prepared and tested in macrophage cells for cancer cell lysis.

Method of Manufacturing Myeloid Cells from a Subject
Myeloid Cell Isolation from PBMCs:

Peripheral blood mononuclear cells can be separated from normal donor buffy coats by density centrifugation using Histopaque 1077 (Sigma). After washing, CD14+ monocytes can be isolated from the mononuclear cell fraction using CliniMACS GMP grade CD14 microbeads and LS separation magnetic columns (Miltenyi Biotec). Briefly, cells can be resuspended to appropriate concentration in PEA buffer (phosphate-buffered saline [PBS] plus 2.5 mmol/L ethylenediaminetetraacetic acid [EDTA] and human serum albumin [0.5% final volume of Alburex 20%, Octopharma]), incubated with CliniMACS CD14 beads per manufacturer's instructions, then washed and passed through a magnetized LS column. After washing, the purified monocytes can be eluted from the demagnetized column, washed and re-suspended in relevant medium for culture. Isolation of CD14+ cells from leukapheresis: PBMCs can be collected by leukapheresis from cirrhotic donors who gave informed consent to participate in the study. Leukapheresis of peripheral blood for mononuclear cells (MNCs) is carried out using an Optia apheresis system by sterile collection. A standard collection program for MNC is used, processing 2.5 blood volumes. Isolation of CD14 cells is carried out using a GMP-compliant functionally closed system (CliniMACS Prodigy system, Miltenyi Biotec). Briefly, the leukapheresis product is sampled for cell count and an aliquot taken for pre-separation flow cytometry. The percentage of monocytes (CD14+) and absolute cell number can be determined, and, if required, the volume is adjusted to meet the required criteria for selection (<20×10 9 total white blood cells; <400×10⁶ white blood cells/mL; <3.5×10⁹ CD14 cells, volume 50-300 mL). CD14 cell isolation and separation is carried out using the CliniMACS Prodigy with CliniMACS CD14 microbeads (medical device class III), TS510 tubing set and LP-14 program. At the end of the process, the selected CD14+ positive monocytes can be washed in PBS/EDTA buffer (CliniMACS buffer, Miltenyi) containing pharmaceutical grade 0.5% human albumin (Alburex), then re-suspended in TexMACS (or comparator) medium for culture.

### Cell Count and Purity:

Cell counts of total MNCs and isolated monocyte fractions can be performed using a Sysmex XP-300 automated analyzer (Sysmex). Assessment of macrophage numbers is carried out by flow cytometry with TruCount tubes (Becton Dickinson) to determine absolute cell number, as the Sysmex consistently underestimated the number of monocytes. The purity of the separation is assessed using flow cytometry (FACSCanto II, BD Biosciences) with a panel of antibodies against human leukocytes (CD45-VioBlue, CD15-FITC, CD14-PE, CD16-APC), and product quality is assessed by determining the amount of neutrophil contamination (CD45int, CD15pos).

Cell Culture—Development of Cultures with Healthy Donor Samples

Optimal culture medium for macrophage differentiation is investigated, and three candidates can be tested using for the cell product. In addition, the effect of monocyte cryopreservation on deriving myeloid cells and macrophages for therapeutic use is examined. Functional assays can be conducted to quantify the phagocytic capacity of myeloid cells and macrophages and their capacity for further polarization, and phagocytic potential as described elsewhere in the disclosure.

Full-Scale Process Validation with Subject Samples

Monocytes cultured from leukapheresis from Prodigy isolation can be cultured at 2×10⁶ monocytes per cm² and per mL in culture bags (MACS GMP differentiation bags, Miltenyi) with GMP-grade TexMACS (Miltenyi) and 100 ng/mL M-CSF. Monocytes can be cultured with 100 ng/mL GMP-compliant recombinant human M-CSF (R&D Systems). Cells can be cultured in a humidified atmosphere at 37° C., with 5% CO₂ for 7 days. A 50% volume media replenishment is carried out twice during culture (days 2 and 4) with 50% of the culture medium removed, then fed with fresh medium supplemented with 200 ng/mL M-CSF (to restore a final concentration of 100 ng/mL).

### Cell Harvesting:

For normal donor-derived macrophages, cells can be removed from the wells at day 7 using Cell Dissociation Buffer (Gibco, Thermo Fisher) and a pastette. Cells can be resuspended in PEA buffer and counted, then approximately 1×10⁶ cells per test can be stained for flow cytometry. Leukapheresis-derived macrophages can be removed from the culture bags at day 7 using PBS/EDTA buffer (CliniMACS buffer, Miltenyi) containing pharmaceutical grade 0.5% human albumin from serum (HAS; Alburex). Harvested cells can be resuspended in excipient composed of two licensed products: 0.9% saline for infusion (Baxter) with 0.5% human albumin (Alburex).

### Flow Cytometry Characterization:

Monocyte and macrophage cell surface marker expression can be analyzed using either a FACSCanto II (BD Biosciences) or MACSQuant 10 (Miltenyi) flow cytometer. Typically, approximately 20,000 events can be acquired for each sample. Cell surface expression of leukocyte markers in freshly isolated and day 7 matured cells is carried out by incubating cells with specific antibodies (final dilution 1:100). Cells are incubated for 5 min with FcR block (Miltenyi) then incubated at 4° C. for 20 min with antibody cocktails. Cells can be washed in PEA, and dead cell exclusion dye DRAQ7 (BioLegend) is added at 1:100. Cells can be stained for a range of surface markers as follows: CD45-VioBlue, CD14-PE or CD14-PerCP-Vio700, CD163-FITC, CD169-PE and CD16-APC (all Miltenyi), CCR2-BV421, CD206-FITC, CXCR4-PE and CD115-APC (all BioLegend), and 25F9-APC and CD115-APC (eBioscience). Both monocytes and macrophages can be gated to exclude debris, doublets and dead cells using forward and side scatter and DRAQ7 dead cell discriminator (BioLegend) and analyzed using FlowJo softwcan be (Tree Star). From the initial detailed phenotyping, a panel is developed as Release Criteria (CD45-VB/CD206-FITC/CD14-PE/25F9 APC/DRAQ7) that defined the development of a functional macrophage from monocytes. Macrophages can be determined as having mean fluorescence intensity (MFI) five times higher than the level on day 0 monocytes for both 25F9 and CD206. A second panel is developed which assessed other markers as part of an Extended Panel, composed of CCR2-BV421/CD163-FITC/CD169-PE/CD14-PerCP-Vio700/CD16-APC/DRAQ7), but is not used as part of the Release Criteria for the cell product.

Monocytes and macrophages can be isolated from withdrawing a buffy coat layer formed in a sucrose gradient centrifugation sample of isolated peripheral blood cells. CD14 cells can be tested for phagocytic uptake using pHRodo beads, which fluoresce only when taken into acidic endosomes. Briefly, monocytes or macrophages can be cultured with 1-2 uL of pHRodo Escherichia coli bioparticles (Life Technologies, Thermo Fisher) for 1 h, then the medium is taken off and cells are washed to remove non-phagocytosed particles. Phagocytosis is assessed using an EVOS microscope (Thermo Fisher), images captured and cellular uptake of beads quantified using ImageJ software (MH). The capacity to polarize toward defined differentiated macrophages is examined by treating day 7 macrophages with IFNγ (50 ng/mL) or IL-4 (20 ng/mL) for 48 h to induce polarization to M1 or M2 phenotype (or M[IFNγ] versus M[IL-4], respectively). After 48 h, the cells can be visualized by EVOS bright-field microscopy, then harvested and phenotyped as before. Further analysis is performed on the cytokine and growth factor secretion profile of macrophages after generation and in response to inflammatory stimuli. Macrophages can be generated from healthy donor buffy coats as before, and either left untreated or stimulated with TNFα (50 ng/mL, Peprotech) and polyinosinic:polycytidylic acid (poly I:C, a viral homolog which binds TLR3, 1 g/mL, Sigma) to mimic the conditions present in the inflamed liver, or lipopolysaccharide (LPS, 100 ng/mL, Sigma) plus IFNγ (50 IU/mL, Peprotech) to produce a maximal macrophage activation. Day 7 macrophages can be incubated overnight and supernatants collected and spun down to remove debris, then stored at −80° C. until testing. Secretome analysis is performed using a 27-plex human cytokine kit and a 9-plex matrix metalloprotease kit run on a Magpix multiplex enzyme linked immunoassay plate reader (BioRad).

### Product Stability:

Various excipients can be tested during process development including PBS/EDTA buffer; PBS/EDTA buffer with 0.5% HAS (Alburex), 0.9% saline alone or saline with 0.5% HAS. The 0.9% saline (Baxter) with 0.5% HAS excipient is found to maintain optimal cell viability and phenotype (data not shown). The stability of the macrophages from cirrhotic donors after harvest is investigated in three process optimization runs, and a more limited range of time points assessed in the process validation runs (n=3). After harvest and re-suspension in excipient (0.9% saline for infusion, 0.5% human serum albumin), the bags can be stored at ambient temperature (21-22° C.) and samples taken at 0, 2, 4, 6, 8, 12, 24, 30 and 48 h postharvest. The release criteria antibody panel is run on each sample, and viability and mean fold change from day 0 is measured from geometric MFI of 25F9 and CD206.

### Statistical Analysis:

Results can be expressed as mean±SD. The statistical significance of differences is assessed where possible with the unpaired two-tailed t-test using GraphPad Prism 6. Results can be considered statistically significant when the P value is <0.05.

Also provided herein is a cell comprising a composition described herein, a vector described herein or a polypeptide described herein. In some embodiments, the cell is a phagocytic cell. In some embodiments, the cell is a stem cell derived cell, a myeloid cell, a macrophage, a dendritic cell, a lymphocyte, a mast cell, a monocyte, a neutrophil, a microglia, or an astrocyte. In some embodiments, the cell is an autologous cell. In some embodiments, the cell is an allogeneic cell. In some embodiments, the cell is an M1 cell. In some embodiments, the cell is an M2 cell. In some embodiments, the cell is an M1 macrophage cell. In some embodiments, the cell is an M2 macrophage cell. In some embodiments, the cell is an M1 myeloid cell. In some embodiments, the cell is an M2 myeloid cell.

Also provided herein is a method of treating a disease in a subject in need thereof comprising administering to the subject a pharmaceutical composition described herein. In some embodiments, the disease is cancer. In some embodiments, the cancer is a solid cancer. In some embodiments, the solid cancer is selected from the group consisting of ovarian cancer, suitable cancers include ovarian cancer, renal cancer, breast cancer, prostate cancer, liver cancer, brain cancer, lymphoma, leukemia, skin cancer, pancreatic cancer, colorectal cancer, lung cancer. In some embodiments, the cancer is a liquid cancer. In some embodiments, the liquid cancer is leukemia or a lymphoma. In some embodiments, the liquid cancer is a T cell lymphoma. In some embodiments, the disease is a T cell malignancy.

In some embodiments, the method further comprises administering an additional therapeutic agent to the subject. In some embodiments, the additional therapeutic agent is selected from the group consisting of a CD47 agonist, an agent that inhibits Rac, an agent that inhibits Cdc42, an agent that inhibits a GTPase, an agent that promotes F-actin disassembly, an agent that promotes PI3K recruitment to the PFP, an agent that promotes PI3K activity, an agent that promotes production of phosphatidylinositol 3,4,5-trisphosphate, an agent that promotes ARHGAP12 activity, an agent that promotes ARHGAP25 activity, an agent that promotes SH3BP1 activity and any combination thereof.

In some embodiments, administering comprises infusing or injecting. In some embodiments, administering comprises administering directly to the solid cancer. In some embodiments, administering comprises a circRNA-based delivery procedure, anon-particle encapsulated mRNA-based delivery procedure, an mRNA-based delivery procedure, viral-based delivery procedure, particle-based delivery procedure, liposome-based delivery procedure, or an exosome-based delivery procedure. In some embodiments, a CD4+ T cell response or a CD8+ T cell response is elicited in the subject.

Also provided herein is a method of preparing a cell, the method comprising contacting a cell with a composition described herein, a vector described herein or a polypeptide described herein. In some embodiments, contacting comprises transducing. In some embodiments, contacting comprises chemical transfection, electroporation, nucleofection, or viral infection or transduction.

Provided herein is a method for administering a therapeutic comprising any one of the compositions described above. In some embodiments, the therapeutic is administered via a parenteral administration route.

In some embodiments, the therapeutic is administered via intramuscular administration route. In some embodiments, the therapeutic is administered via intravenous administration route. In some embodiments, the therapeutic is administered via subcutaneous administration route.

Also provided herein is a method of preparing a pharmaceutical composition comprising the one or more recombinant nucleic acids described herein and a lipid in an aqueous composition described herein. In some embodiments, the composition comprises a vector described herein. In some embodiments, the lipid comprises forming a lipid nanoparticle.

### EXAMPLES

### Example 1. Generation of a Recombinant Chimeric Protein with Transcription Factor Under Protease Control

In this example, a recombinant polynucleic acid is constructed that comprises a sequence encoding a chimeric fusion protein having an anti-TROP-2 binding domain, a CD89 transmembrane and cytosolic domain; NS4A/4B protease cleavage site; a constitutively active IRF7; a T2A; Syk tandem SH2; and NS3 protease in tandem succession from N-C terminal. For example, the complete sequence of the translated product is a first fusion protein—a CFP that is an anti-TROP-2-CD89-Protease Cleavage site-IRF7TF; this section is post-translationally from the second fusion protein generated cotranslationally, that comprises a Syk Tandem SH2-linker-NS3 protease.

The sequence of the recombinant protein is provided as follows:

(SEQ ID NO: 242)
MWLQSLLLLGTVACSISQVQLQQSGSELKKPGASVKVSCKASGYTFTNYG
MNWVKQAPGQGLKWMGWINTYTGEPTYTDDFKGRFAFSLDTSVSTAYLQI
SSLKADDTAVYFCARGGFGSSYWYFDVWGQGSLVTVSSGGGGSGGGGSGG
GGSDIQLTQSPSSLSASVGDRVSITCKASQDVSIAVAWYQQKPGKAPKLL
IYSASYRYTGVPDRFSGSGSGTDFTLTISSLQPEDFAVYYCQQHYITPLT
FGAGTKVEIKRSGGGGAAAGSDSIHQDYTTQNLIRMAVAGLVLVALLAIL
VENWHSHTALNKEASADVAEPSWSQQMCQPGLTFARTPSVCKGGGSGGGD
EMEECSQHGGGSGGGMALAPERAAPRVLFGEWLLGEISSGCYEGLQWLDE
ARTCFRVPWKHFARKDLSEADARIFKAWAVARGRWPPSSRGGGPPPEAET
AERAGWKTNFRCALRSTRRFVMLRDNSGDPADPHKVYALSRELCWREGPG
TDQTEAEAPAAVPPPQGGPPGPFLAHTHAGLQAPGPLPAPAGDKGDLLLQ
AVQQSCLADHLLTASWGADPVPTKAPGEGQEGLPLTGACAGGPGLPAGEL
YGWAVETTPSPEGVSSLDSSSLSLCLSSANSLYDDIECFLMELEQPAGSG
EGRGSLLTCGDVEENPGPGSGASSGMADSANHLPFFFGNITREEAEDYLV
QGGMSDGLYLLRQSRNYLGGFALSVAHGRKAHHYTIERELNGTYAIAGGR
THASPADLCHYHSQESDGLVCLLKKPFNRPQGVQPKTGPFEDLKENLIRE
YVKQTWNLQGQALEQAIISQKPQLEKLIATTAHEKMPWFHGKISREESEQ
IVLIGSKTNGKFLIRARDNNGSYALCLLHEGKVLHYRIDKDKTGKLSIPE
GKKFDTLWQLVEHYSYKADGLLRVLTVPCGGGSGGGTGCVVIVGRIVLSG
SGTSAPITAYAQQTRGLLGCIITSLTGRDKNQVEGEVQIVSTATQTFLAT
CINGVCWAVYHGAGTRTIASPKGPVIQMYTNVDQDLVGWPAPQGSRSLTP
CTCGSSDLYLVTRHADVIPVRRRGDSRGSLLSPRPISYLKGSSGGPLLCP
AGHAVGLFRAAVCTRGVAKAVDFIPVENLETTMRSPVFTDNSSPPAVTLT
H

The respective domains of the cotranslationally generated CFP-IRF5-Syk-NS3 fusion protein having the sequence described above, are exhibited in Table 5 below.

TABLE 5
Domains and functional information of the
TROP2 construct described above

| Domain or parts | Amino acid sequence |
|---|---|
| Signal Peptide | MWLQSLLLLGTVACSIS |
| | (SEQ ID NO: 219) |
| TROP2 scFv-VH | QVQLQQSGSELKKPGASVKVSCKASGYTFTNYGM |
| (CDR1, CDR2 | NWVKQAPGQGLKWMGWINTYTGEPTYTDDFKGRF |
| and CDR3 | AFSLDTSVSTAYLQISSLKADDTAVYFCARGGFG |
| underlined) | SSYWYFDVWGQGSLVTVSS |
| | (SEQ ID NO: 191) |
| Linker 1 | GGGGSGGGGSGGGGS |
| | (SEQ ID NO: 225) |
| TROP2 scFv-VL | DIQLTQSPSSLSASVGDRVSITCKASQDVSIAVA |
| (CDR1, CDR2 | WYQQKPGKAPKLLIYSASYRYTGVPDRFSGSGSG |
| and CDR3 | TDFTLTISSLQPEDFAVYYCQQHYITPLTFGAGT |
| underlined) | |
| | KVEIKR |
| | (SEQ ID NO: 192) |
| Linker2 | SGGGGAAAGS |
| | (SEQ ID NO: 243) |
| CD89 | DSIHQDYTTQNLIRMAVAGLVLVALLAILVENWH |
| transmembrane | SHTALNKEASADVAEPSWSQQMCQPGLTFARTPS |
| and cytosolic | VCK |
| domain | (SEQ ID NO: 197) |
| Linker 3A | GGGSGGG |
| | (SEQ ID NO: 244) |
| NS4A/4B | DEMEECSQH |
| protease | (SEQ ID NO: 232) |
| cleavage site | |
| Linker 3B | GGGSGGG |
| | (SEQ ID NO: 244) |
| Constitutively | MALAPERAAPRVLFGEWLLGEISSGCYEGLQWLD |
| Active IRF7 | EARTCFRVPWKHFARKDLSEADARIFKAWAVARG |
| | RWPPSSRGGGPPPEAETAERAGWKTNFRCALRST |
| | RRFVMLRDNSGDPADPHKVYALSRELCWREGPGT |
| | DQTEAEAPAAVPPPQGGPPGPFLAHTHAGLQAPG |
| | PLPAPAGDKGDLLLQAVQQSCLADHLLTASWGAD |
| | PVPTKAPGEGQEGLPLTGACAGGPGLPAGELYGW |
| | AVETTPSPEGVSSLDSSSLSLCLSSANSLYDDIE |
| | CFLMELEQPAGSG |
| | (SEQ ID NO: 245) |
| T2A | EGRGSLLTCGDVEENPGP |
| | (SEQ ID NO: 246) |
| Linker 4 | GSG |
| Syk Tandem SH2 | ASSGMADSANHLPFFFGNITREEAEDYLVQGGMS |
| | DGLYLLRQSRNYLGGFALSVAHGRKAHHYTIERE |
| | LNGTYAIAGGRTHASPADLCHYHSQESDGLVCLL |
| | KKPFNRPQGVQPKTGPFEDLKENLIREYVKQTWN |
| | LQGQALEQAIISQKPQLEKLIATTAHEKMPWFHG |
| | KISREESEQIVLIGSKTNGKFLIRARDNNGSYAL |
| | CLLHEGKVLHYRIDKDKTGKLSIPEGKKFDTLWQ |
| | LVEHYSYKADGLLRVLTVPC |
| | (SEQ ID NO: 247) |
| Linker 5 | GGGSGGG |
| | (SEQ ID NO: 244) |
| NS3 protease | TGCVVIVGRIVLSGSGTSAPITAYAQQTRGLLGC |
| | IITSLTGRDKNQVEGEVQIVSTATQTFLATCING |
| | VCWAVYHGAGTRTIASPKGPVIQMYTNVDQDLVG |
| | WPAPQGSRSLTPCTCGSSDLYLVTRHADVIPVRR |
| | RGDSRGSLLSPRPISYLKGSSGGPLLCPAGHAVG |
| | LFRAAVCTRGVAKAVDFIPVENLETTMRSPVFTD |
| | NSSPPAVTLTH |
| | (SEQ ID NO: 248) |

1. A composition comprising a recombinant polynucleic acid, wherein the recombinant polynucleic acid comprises a sequence encoding a cell surface receptor, wherein the cell surface receptor is a chimeric fusion protein (CFP) comprising:
(a) an extracellular domain comprising an antigen binding domain,
(b) a transmembrane domain operatively linked to the extracellular domain, wherein the transmembrane domain is a transmembrane domain from a protein that dimerizes with Fc receptor γ-chain, and wherein the CFP undergoes degradation when expressed in a cell that does not express Fc receptor γ-chain; and
(c) an intracellular domain operatively linked to the transmembrane domain, the intracellular domain comprising:
(i) a therapeutic agent, wherein the therapeutic agent is a transcription factor or transcriptionally functional portion thereof, and
(ii) a protease cleavage site disposed between the transcription factor and the transmembrane domain.

2. The composition of claim 1, wherein the recombinant polynucleic acid is expressed in a cell that naturally expresses an Fc receptor γ-chain.

3. The composition of claim 1, wherein cleavage of the protease cleavage site by a protease releases the therapeutic agent from the CFP.

4. (canceled)

5. The composition of claim 1, wherein the transcription factor is an inflammatory response transcription factor.

6. The composition of claim 5, wherein the transcription factor is IRF3, IRF5 or IRF7.

7. (canceled)

8. The composition of claim 1, wherein the intracellular domain of the CFP lacks a phosphorylated tyrosine residue.

9. The composition of claim 1, wherein the intracellular domain of the CFP lacks a phosphotyrosine binding (PTB) domain.

10. The composition of claim 1, wherein the protease cleavage site is a viral protease cleavage site.

11. The composition of claim 10, wherein the viral protease cleavage site is for a viral protease derived from hepatitis C virus (HCV) nonstructural protein 3 (NS3).

12. The composition of claim 10, wherein the viral protease cleavage site is selected from the group consisting of: an NS4A/4B junction cleavage site, an NS3/NS4A junction cleavage site, an NS4A/NS4B junction cleavage site, an NS4B/NS5A junction cleavage site, an NS5A/NS5B junction cleavage site, and variants thereof cleavable by the viral protease.

13. The composition of claim 1, wherein the protease cleavage site is cleaved by a protease upon activation of the CFP, in a cell expressing Fc receptor γ-chain.

14. (canceled)

15. (canceled)

16. The composition of claim 1, wherein the recombinant polynucleic acid further comprises a sequence encoding an additional fusion protein, comprising a phosphotyrosine binding (PTB) domain connected to a protease.

17. (canceled)

18. (canceled)

19. The composition of claim 16, wherein the additional fusion protein is a soluble cytosolic fusion protein.

20. The composition of claim 16, wherein the sequence encoding the additional fusion protein is separated from the sequence encoding the CFP by an auto-cleavable sequence.

21. The composition of claim 16, wherein the additional fusion protein is intracellularly tethered to a cell membrane when expressed in a cell.

22. The composition of claim 21, wherein the second additional fusion protein is intracellularly tethered to the cell membrane:
(i) via a transmembrane domain, or dimerization domain that dimerizes with Fc receptor γ-chain,
(ii) via a dimerization domain that comprises a leucine zipper domain, a helix-loop-helix domain, or both; or
(iii) via an anchor, wherein the anchor is a glycolipid anchor.

23. The composition of claim 16, wherein the sequence encoding the additional fusion protein further comprises a sequence encoding a dimerization domain that dimerizes with a domain of a cell surface receptor to promote association of the protease and the cell surface receptor.

24-32. (canceled)

33. The composition of claim 16, wherein the second additional fusion protein further comprises a degron, wherein degradation activity of the degron is inhibited by binding of the PTB domain of the fusion protein to a phosphorylated tyrosine residue, wherein the phosphorylated tyrosine residue is on an endogenous receptor of a cell that is not constitutively phosphorylated at the tyrosine residue.

34. (canceled)

35. (canceled)

36. The composition of claim 33, wherein the endogenous receptor is phosphorylated at the tyrosine residue in cells expressing the CFP that are bound to a diseased cell expressing an antigen recognized by the antigen binding domain of the CFP.

37-39. (canceled)

40. The composition of claim 1, wherein the antigen binding domain is a CD5 binding domain, a HER2 binding domain, a GPC3 binding domain, or a TROP2 binding domain.

41-45. (canceled)

46. The composition of claim 1, wherein the transmembrane domain of the CFP is a transmembrane domain from CD16a, CD64, CD68 or CD89.

47-59. (canceled)

60. The composition of claim 1, wherein the recombinant polynucleic acid is an mRNA.

61-66. (canceled)

67. The composition of claim 1, wherein the composition further comprises an inhibitor of the protease.

68. The composition of claim 67, wherein the inhibitor of the protease is selected from the group consisting of: asunaprevir (ASV), danoprevir (DPV), simeprevir (SPV), grazoprevir (GPV), and any combination thereof.

69-82. (canceled)

83. A pharmaceutical composition comprising the composition of claim 16, wherein the recombinant polynucleic acid is mRNA, and a nanoparticle delivery vehicle encapsulating the mRNA.

84-87. (canceled)

88. A method of treating a cancer in a human subject in need thereof, comprising administering to the human subject the pharmaceutical composition of claim 83.

89-92. (canceled)


SEQUENCE LISTING (248 sequences)

SEQ ID NO: 1 - protein, 193 aa, Hepatitis C virus
APITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAV

SEQ ID NO: 2 - protein, 14 aa, synthetic construct
ATACADTTQYAYDY

SEQ ID NO: 3 - protein, 14 aa, synthetic construct
ATACADTTLYEYDY

SEQ ID NO: 4 - protein, 14 aa, synthetic construct
ATACVDTTQYEYDY

SEQ ID NO: 5 - protein, 14 aa, synthetic construct
ATACADATQHEYDY

SEQ ID NO: 6 - protein, 14 aa, synthetic construct
ATACADTTQYDYDY

SEQ ID NO: 7 - protein, 14 aa, synthetic construct
ATACADTTQYEYDY

SEQ ID NO: 8 - protein, 14 aa, synthetic construct
ATACADTTHYEYDY

SEQ ID NO: 9 - protein, 14 aa, synthetic construct
ATACVITTLYEYDY

SEQ ID NO: 10 - protein, 14 aa, synthetic construct
ATACAETTLYEYDY

SEQ ID NO: 11 - protein, 14 aa, synthetic construct
ATACADTTQHEYDY

SEQ ID NO: 12 - protein, 14 aa, synthetic construct
ATACVDTTHYEYDY

SEQ ID NO: 13 - protein, 14 aa, synthetic construct
ATACASTTLYEYDY

SEQ ID NO: 14 - protein, 14 aa, synthetic construct
ATACVVTTLYEYDY

SEQ ID NO: 15 - protein, 13 aa, synthetic construct
ATACGGATGPYDY

SEQ ID NO: 16 - protein, 13 aa, synthetic construct
ATACAGAIGPYDY

SEQ ID NO: 17 - protein, 13 aa, synthetic construct
ATACVVVGDQNDY

SEQ ID NO: 18 - protein, 13 aa, synthetic construct
ATACVVVGDRNDY

SEQ ID NO: 19 - protein, 14 aa, synthetic construct
ATDCAGGTSTPYDY

SEQ ID NO: 20 - protein, 14 aa, synthetic construct
ATDCAGGTATPYDY

SEQ ID NO: 21 - protein, 14 aa, synthetic construct
ATACVVADRNEYDY

SEQ ID NO: 22 - protein, 14 aa, synthetic construct
ATSCVVVTKNEYDY

SEQ ID NO: 23 - protein, 13 aa, synthetic construct
ATACSGLTHEYDY

SEQ ID NO: 24 - protein, 13 aa, synthetic construct
ATTCSGLTHEYDY

SEQ ID NO: 25 - protein, 15 aa, synthetic construct
ATACANWSSLGPYDY

SEQ ID NO: 26 - protein, 15 aa, synthetic construct
ATACANWSTLGPYDY

SEQ ID NO: 27 - protein, 14 aa, synthetic construct
ATACSDPRVYEYDY

SEQ ID NO: 28 - protein, 14 aa, synthetic construct
ATTCASPEKYEYDY

SEQ ID NO: 29 - protein, 15 aa, synthetic construct
ATHCGGTSWGTSYDY

SEQ ID NO: 30 - protein, 15 aa, synthetic construct
ATHCGGSSWSNEYDY

SEQ ID NO: 31 - protein, 9 aa, synthetic construct
YARYSGRTY

SEQ ID NO: 32 - protein, 17 aa, synthetic construct
ASSAWPAGPKHQVEYDY

SEQ ID NO: 33 - protein, 13 aa, synthetic construct
ATACGSLVGMYDY

SEQ ID NO: 34 - protein, 13 aa, synthetic construct
ATACGSAVHEYDY

SEQ ID NO: 35 - protein, 14 aa, synthetic construct
ATDCVGFGSNWFDY

SEQ ID NO: 36 - protein, 14 aa, synthetic construct
ATACASPVIYEYDY

SEQ ID NO: 37 - protein, 14 aa, synthetic construct
ATDCAGGVGHEYDY

SEQ ID NO: 38 - protein, 15 aa, synthetic construct
ATDCSLHGSDYPYDY

SEQ ID NO: 39 - protein, 17 aa, synthetic construct
AVRIYSGSFDNTLAYDY

SEQ ID NO: 40 - protein, 8 aa, synthetic construct
GFPLAYYA

SEQ ID NO: 41 - protein, 8 aa, synthetic construct
GFSLDYYA

SEQ ID NO: 42 - protein, 8 aa, synthetic construct
GFPLDYYA

SEQ ID NO: 43 - protein, 8 aa, synthetic construct
GFTLDYYA

SEQ ID NO: 44 - protein, 8 aa, synthetic construct
GFSLNYYA

SEQ ID NO: 45 - protein, 8 aa, synthetic construct
GFTLAYYA

SEQ ID NO: 46 - protein, 8 aa, synthetic construct
GFTLGYYA

SEQ ID NO: 47 - protein, 8 aa, synthetic construct
GFPLNYYA

SEQ ID NO: 48 - protein, 8 aa, synthetic construct
GFPLHYYA

SEQ ID NO: 49 - protein, 8 aa, synthetic construct
GFSLGYYA

SEQ ID NO: 50 - protein, 8 aa, synthetic construct
GFPLGYYA

SEQ ID NO: 51 - protein, 8 aa, synthetic construct
GFPLEYYA

SEQ ID NO: 52 - protein, 8 aa, synthetic construct
GSDFRADA

SEQ ID NO: 53 - protein, 8 aa, synthetic construct
GRTFSSYG

SEQ ID NO: 54 - protein, 8 aa, synthetic construct
GFSLAYYA

SEQ ID NO: 55 - protein, 8 aa, synthetic construct
GLTFRSVG

SEQ ID NO: 56 - protein, 8 aa, synthetic construct
ISNSDGST

SEQ ID NO: 57 - protein, 8 aa, synthetic construct
ISASDGST

SEQ ID NO: 58 - protein, 8 aa, synthetic construct
ISSSDGST

SEQ ID NO: 59 - protein, 8 aa, synthetic construct
ISSSDGNT

SEQ ID NO: 60 - protein, 8 aa, synthetic construct
ISSADGST

SEQ ID NO: 61 - protein, 8 aa, synthetic construct
ISSSGGST

SEQ ID NO: 62 - protein, 8 aa, synthetic construct
ISSGDGST

SEQ ID NO: 63 - protein, 8 aa, synthetic construct
ISAGDGNT

SEQ ID NO: 64 - protein, 8 aa, synthetic construct
ISSSDDST

SEQ ID NO: 65 - protein, 8 aa, synthetic construct
ISSNDGST

SEQ ID NO: 66 - protein, 8 aa, synthetic construct
ISSPDGST

SEQ ID NO: 67 - protein, 8 aa, synthetic construct
ISSRTGGT

SEQ ID NO: 68 - protein, 9 aa, synthetic construct
ISAGDGSST

SEQ ID NO: 69 - protein, 13 aa, synthetic construct
ISSSDGSSSDGNT

SEQ ID NO: 70 - protein, 8 aa, synthetic construct
ISSGDGNT

SEQ ID NO: 71 - protein, 8 aa, synthetic construct
ISSGDGKT

SEQ ID NO: 72 - protein, 8 aa, synthetic construct
ISSSDGGT

SEQ ID NO: 73 - protein, 8 aa, synthetic construct
ISSRTGST

SEQ ID NO: 74 - protein, 8 aa, synthetic construct
ISSRTGNT

SEQ ID NO: 75 - protein, 10 aa, synthetic construct
ISSSDGHSST

SEQ ID NO: 76 - protein, 9 aa, synthetic construct
ISSSSDGNT

SEQ ID NO: 77 - protein, 8 aa, synthetic construct
ISASNGNT

SEQ ID NO: 78 - protein, 9 aa, synthetic construct
ISSGSDGNT

SEQ ID NO: 79 - protein, 8 aa, synthetic construct
ISASDGNT

SEQ ID NO: 80 - protein, 7 aa, synthetic construct
IDSITSI

SEQ ID NO: 81 - protein, 16 aa, synthetic construct
ISWSGGSTIAASVGST

SEQ ID NO: 82 - protein, 11 aa, synthetic construct
ISSSDGSDGNT

SEQ ID NO: 83 - protein, 8 aa, synthetic construct
ASPSGVIT

SEQ ID NO: 84 - protein, 121 aa, synthetic construct
QVQLQESGGGLVHSGGSLRLSCAASGFPLAYYAIGWFRQAPGKEREGVSCISSSDGNTYYADAVKGRFTISRDNAKNTVYLQMNSLKPEDTAVYYCATACADTTQHEYDYWGQGTQVTVSS

SEQ ID NO: 85 - protein, 121 aa, synthetic construct
QVQLQESGGGLVHSGGSLRLSCAASGFPLDYYAIGWFRQAPGKEREGVSCISSADGSTYYADSVKGRFTISRDNAKNTVYLQMNSLGPEDTAVYYCATACADTTQYDYDYWGQGTQVTVSS

SEQ ID NO: 86 - protein, 120 aa, synthetic construct
QVQLQESGGGLVHSGGSLRLSCAASGFTLDYYAIGWFRRAPGKEREGVSCISSGDGKTYYADSVKGRFTISRDNAKNTVYLQMNSLKPEDTAVYYCATACAGAIGPYDYWGQGTQVTVSS

SEQ ID NO: 87 - protein, 121 aa, synthetic construct
QVQLQESGGGLVPPGGSLRLSCAASGFPLDYYAIGWFRQAPGKEREGVSCISSADGSTYYADSVKGRFTISRDNAKNTVYLQMNSLGPEDTAVYYCATACADTTQYDYDYWGQGTQVTVSS

SEQ ID NO: 88 - protein, 123 aa, synthetic construct
QVQLQESGGGLVQAGGSLRLSCAASGFSLGYYAIGWFRQAPGKEREGVSCISSSDGHSSTYYADSVKGRFTISRDNAKNTVYLQMNNLKPEDTAVYYCATDCAGGTATPYDYWGQGTQVTVSS

SEQ ID NO: 89 - protein, 124 aa, synthetic construct
QVQLQESGGGLVQAGGSLRLSCAASGRTFSSYGMGWFRQAPGKEREFVAAISWSGGSTYYADSVKGRFTISRDNAKNTVYLQMNSLKPEDTAVYYCASSAWPAGPKHQVEYDYWGQGTQVTVSS

SEQ ID NO: 90 - protein, 120 aa, synthetic construct
QVQLQESGGGLVQAGGSLRLSCTASGFSLDYYAIGWFRQAPGKEREGVACISSRTGSTYYADSVKGRFTISRDNAKNTVALQMNSLKPEDTAVYYCATACVVVGDQNDYWGQGTQVTVSS

SEQ ID NO: 91 - protein, 121 aa, synthetic construct
QVQLQESGGGLVQDGGSLRLSCAASGFPLAYYAIGWFRQAPGKEREGVSCISASDGSTYYADSVKGRFTISRDNAKNTVYLQMNSLKPEDTAVYYCATACAETTLYEYDYWGQGTQVTVSS

SEQ ID NO: 92 - protein, 122 aa, synthetic construct
QVQLQESGGGLVQPGESLRLSCAASGFPLAYYAIGWFRQAPGKEREGVSCISSSDGSTYYADSVKGRFTISRDNAKNTVYLQMNSLKPEDTAVYYCATACANWSTLGPYDYWGQGTQVTVSS

SEQ ID NO: 93 - protein, 121 aa, synthetic construct
QVQLQESGGGLVQPGESLRLSCAASGFTLAYYAIGWFRQAPGKEREGVSCISSSDGNTYYADSVKGRFTISRDNAKNTVYLQMNRLKPEDTAVYYCATACADTTQYEYDYWGQGTQVTVSS

SEQ ID NO: 94 - protein, 114 aa, synthetic construct
QVQLQESGGGLVQPGGSLKLSCAASGSDFRADAMGWYRQAPGKEREPVAIDSITSIYYVDSVEGRFTISRDNTKNTVYLQMTSLKPEDTAVYYCYARYSGRTYWGRGTQVTVSS

SEQ ID NO: 95 - protein, 121 aa, synthetic construct
QVQLQESGGGLVQPGGSLRLSCAASGFPLAYYAIGWFRQAPGKEREGVSCISASDGSTYYADSVKGRFTISRDNAKNTVYLQMNSLRPEDTAVYYCATACADTTLYEYDYWGQGTQVTVSS

SEQ ID NO: 96 - protein, 121 aa, synthetic construct
QVQLQESGGGLVQPGGSLRLSCAASGFPLAYYAIGWFRQAPGKEREGVSCISSSDGNTYYADAVKGRFAISRDNAKNTVYLQMNSLKPEDTAVYYCATACSDPRVYEYDYWGQGTQVTVSS

SEQ ID NO: 97 - protein, 121 aa, synthetic construct
QVQLQESGGGLVQPGGSLRLSCAASGFPLAYYAIGWFRQAPGKEREGVSCISSSDGNTYYADAVKGRFTISRDNAKNTVYLQMNSLKPEDTAVYYCATACADTTQHEYDYWGQGTQVTVSS

SEQ ID NO: 98 - protein, 121 aa, synthetic construct
QVQLQESGGGLVQPGGSLRLSCAASGFPLAYYAIGWFRQAPGKEREGVSCISSSDGNTYYADAVKGRFTISRDNAKNTVYLQMNSLKPEDTAVYYCATACVDTTHYEYDYWGQGTQVTVSS

SEQ ID NO: 99 - protein, 121 aa, synthetic construct
QVQLQESGGGLVQPGGSLRLSCAASGFPLAYYAIGWFRQAPGKEREGVSCISSSDGNTYYADSVKGRFTISRDNAKNTVYLQMNSLKPEDTAVYYCATACADATQHEYDYWGQGTQVTVSS

SEQ ID NO: 100 - protein, 121 aa, synthetic construct
QVQLQESGGGLVQPGGSLRLSCAASGFPLAYYAIGWFRQAPGKEREGVSCISSSDGSTYYADSVKGRFTISRDNAKNTVYLQMNSLGPEDTAVYYCATACADTTQYDYDYWGQGTQVTVSS

SEQ ID NO: 101 - protein, 121 aa, synthetic construct
QVQLQESGGGLVQPGGSLRLSCAASGFPLAYYAIGWFRQAPGKEREGVSCISSSDGSTYYADSVKGRFTISRDNAKNTVYLQMNSLKPEDTAVYYCATACADTTQYEYDYWGQGTQVTVSS

SEQ ID NO: 102 - protein, 120 aa, synthetic construct
QVQLQESGGGLVQPGGSLRLSCAASGFPLAYYAIGWFRQAPGKEREGVSCISSSDGSTYYADSVKGRFTISRDNAKNTVYLQMNSLKPEDTAVYYCATACGGATGPYDYWGQGTQVTVSS

SEQ ID NO: 103 - protein, 121 aa, synthetic construct
QVQLQESGGGLVQPGGSLRLSCAASGFPLAYYAIGWFRRAPGKEREGVSCISSSDGNTYYADAVKGRFTISRDNAKNTVYLQMNSLKPEDTAVYYCATACADTTQHEYDYWGQGTQVTVSS

SEQ ID NO: 104 - protein, 122 aa, synthetic construct
QVQLQESGGGLVQPGGSLRLSCAASGFPLDYYAIGWFRQAPGKEREGVSCISAGDGSSTYYADSVKGRFTISRDNAKNTVYLQMNSLKPEDTAVYYCATACASTTLYEYDYWGQGTQVTVSS

SEQ ID NO: 105 - protein, 121 aa, synthetic construct
QVQLQESGGGLVQPGGSLRLSCAASGFPLDYYAIGWFRQAPGKEREGVSCISSADGSTYYADSVKGRFTISRDNAKNAVYLQMNSLGPEDTAVYYCATACADTTQYDYDYWGQGTQVTVSS

SEQ ID NO: 106 - protein, 121 aa, synthetic construct
QVQLQESGGGLVQPGGSLRLSCAASGFPLDYYAIGWFRQAPGKEREGVSCISSADGSTYYADSVKGRFTISRDNAKNTVYLQMNSLGPEDTAVYYCATACADTTQYDYDYWGQGTQVTVSS

SEQ ID NO: 107 - protein, 121 aa, synthetic construct
QVQLQESGGGLVQPGGSLRLSCAASGFPLDYYAIGWFRQAPGKEREGVSCISSADGSTYYADSVKGRFTISRDNAKNTVYLQMNSLKPEDTAVYYCATACVDTTQYEYDYWGQGTQVTVSS

SEQ ID NO: 108 - protein, 121 aa, synthetic construct
QVQLQESGGGLVQPGGSLRLSCAASGFPLDYYAIGWFRQAPGKEREGVSCISSPDGSTYYADSVKGRFTISRDNAKNTVYLQMNSLKPEDTAVYYCATACVDTTQYEYDYWGQGTQVTVSS

SEQ ID NO: 109 - protein, 125 aa, synthetic construct
QVQLQESGGGLVQPGGSLRLSCAASGFPLDYYAIGWFRQAPGKEREGVSCISSSDGSDGNTYYADSVKGRFTISRDNAKNTVYLQMNSLKPEDTAVYYCATDCSLHGSDYPYDYWGQGTQVTVSS

SEQ ID NO: 110 - protein, 121 aa, synthetic construct
QVQLQESGGGLVQPGGSLRLSCAASGFPLDYYAIGWFRQAPGKEREGVSCISSSDGSTYYADSVKGRFTISRDNAKNTVYLQMNSLKPEDTAVYYCATACADTTQYEYDYWGQGTQVTVSS

SEQ ID NO: 111 - protein, 121 aa, synthetic construct
QVQLQESGGGLVQPGGSLRLSCAASGFPLEYYAIGWFRQAPGKEREGVSCISSSDGSTYYADSVKGRFTISRDNAKNTVYLQMNSLKPEDTAVYYCATACSDPRVYEYDYWGQGTQVTVSS

SEQ ID NO: 112 - protein, 121 aa, synthetic construct
QVQLQESGGGLVQPGGSLRLSCAASGFPLGYYAIGWFRQAPGKEREGVSCISSSDDSTYYADSVKGRFTISRDNDKNTVYLQMNSLKPEDTAVYYCATDCAGGTSTPYDYWGQGTQVTVSS

SEQ ID NO: 113 - protein, 121 aa, synthetic construct
QVQLQESGGGLVQPGGSLRLSCAASGFPLHYYAIGWFRQAPGKEREGVSCISSGDGSTYYADSVKGRFTISRDNAKNTVYLQMNSLKPEDTAVYYCATSCVVVTKNEYDYWGQGTQVTVSS

SEQ ID NO: 114 - protein, 120 aa, synthetic construct
QVQLQESGGGLVQPGGSLRLSCAASGFPLHYYAIGWFRQAPGKEREGVSCISSSDGSTYYADSVKGRFTISRDNAKNTVYLQMNSLKPEDTAVYYCATACGGATGPYDYWGQGTQVTVSS

SEQ ID NO: 115 - protein, 121 aa, synthetic construct
QVQLQESGGGLVQPGGSLRLSCAASGFPLHYYAIGWFRQAPGKEREGVSCISSSDGSTYYADSVKGRFTISRDNAKNTVYLQMNSLKPEDTAVYYCATACVVADRNEYDYWGQGTQVTVSS

SEQ ID NO: 116 - protein, 121 aa, synthetic construct
QVQLQESGGGLVQPGGSLRLSCAASGFPLHYYAIGWFRQAPGKEREGVSCISSSDGSTYYADSVKGRFTISRDNAKNTVYLQMNSLRPEDTAVYYCATACVVADRNEYDYWGQGTQVTVSS

SEQ ID NO: 117 - protein, 121 aa, synthetic construct
QVQLQESGGGLVQPGGSLRLSCAASGFPLNYYAIGWFRQAPGKEREGVSCISASDGNTYYADSVKGRFTISRDNAKNTVYLQMNSLKPEDTAVYYCATTCASPEKYEYDYWGQGTQVTVSS

SEQ ID NO: 118 - protein, 120 aa, synthetic construct
QVQLQESGGGLVQPGGSLRLSCAASGFPLNYYAIGWFRQAPGKEREGVSCISSSDGSTYYADSVKGRFIISRDNAKNTVYLQMNSLKPEDTAVYYCATACGGATGPYDYWGQGTQVTVSS

SEQ ID NO: 119 - protein, 120 aa, synthetic construct
QVQLQESGGGLVQPGGSLRLSCAASGFPLNYYAIGWFRQAPGKEREGVSCISSSDGSTYYADSVKGRFTISRDNAKNTVYLQMNSLKPEDTAVYYCATACGSAVHEYDYWGQGTQVTVSS

SEQ ID NO: 120 - protein, 121 aa, synthetic construct
QVQLQESGGGLVQPGGSLRLSCAASGFSLAYYAIGWFRQAPGKEREGVSCIAASVGSTYYADSVKGRFTISRDDAKNTVYLQMNSLKPEDTAVYYCATDCAGGVGHEYDYWGQGTQVTVSS

SEQ ID NO: 121 - protein, 120 aa, synthetic construct
QVQLQESGGGLVQPGGSLRLSCAASGFSLDYYAIGWFRQAPGKEREGVSCISSSDGSTYYADSVKGRFTISRDNAKNAVYLQMNSLKPEDTAVYYCATACGGATGPYDYWGQGTQVTVSS

SEQ ID NO: 122 - protein, 121 aa, synthetic construct
QVQLQESGGGLVQPGGSLRLSCAASGFSLDYYAIGWFRQAPGKEREGVSCISSSDGSTYYADSVKGRFTISRDNAKNAVYLQMNSLKPEDTAVYYCATACVDTTQYEYDYWGQGTQVTVSS

SEQ ID NO: 123 - protein, 121 aa, synthetic construct
QVQLQESGGGLVQPGGSLRLSCAASGFSLDYYAIGWFRQAPGKEREGVSCISSSDGSTYYADSVKGRFTISRDNAKNTVYLQMNSLKPEDTAVYYCATDCAGGTSTPYDYWGQGTQVTVSS

SEQ ID NO: 124 - protein, 121 aa, synthetic construct
QVQLQESGGGLVQPGGSLRLSCAASGFSLNYYAIGWFRQAPGKEREGVSCISAGDGNTYYADSVKGRFTISRDNAANTVSLQMDSLKPEDTAVYYCATACVITTLYEYDYWGQGTQVTVSS

SEQ ID NO: 125 - protein, 121 aa, synthetic construct
QVQLQESGGGLVQPGGSLRLSCAASGFTLAYYAIGWFRQAPGKEREGVSCISSSDGSTYYADSVKGRFTISRDNAKNTVYLQMNSLKPEDTAVYYCATACADTTQHEYDYWGQGTQVTVSS

SEQ ID NO: 126 - protein, 121 aa, synthetic construct
QVQLQESGGGLVQPGGSLRLSCAASGFTLAYYAIGWFRQAPGKEREGVSCISSSDGSTYYADSVKGRFTISRDNAKNTVYLQMNSLKPEDTAVYYCATACADTTQYEYDYWGQGTQVTVSS

SEQ ID NO: 127 - protein, 120 aa, synthetic construct
QVQLQESGGGLVQPGGSLRLSCAASGFTLDYYAIGWFRQAPGKEREGVACISSSDGSTYYADSVKGRFTISRDNAKNTVYLQMNSLKPEDTAVYYCATACGGATGPYDYWGQGTQVTVSS

SEQ ID NO: 128 - protein, 120 aa, synthetic construct
QVQLQESGGGLVQPGGSLRLSCAASGFTLDYYAIGWFRQAPGKEREGVACISSSDGSTYYADSVKGRFTISRDNAKNTVYLQMNSLKPQDTAVYYCATACGSLVGMYDYWGQGTQVTVSP

SEQ ID NO: 129 - protein, 121 aa, synthetic construct
QVQLQESGGGLVQPGGSLRLSCAASGFTLDYYAIGWFRQAPGKEREGVSCISASDGNTYYADSVKGRFTISRDNAKNTVYLQMNSLKPEDTAVYYCATTCASPEKYEYDYWGQGTQVTVSS

SEQ ID NO: 130 - protein, 120 aa, synthetic construct
QVQLQESGGGLVQPGGSLRLSCAASGFTLDYYAIGWFRQAPGKEREGVSCISASNGNTYYADSVKGRFTISRDSAKNTVYLQMNSLKPEDTAVYYCATTCSGLTHEYDYWGQGTQVTVSS

SEQ ID NO: 131 - protein, 120 aa, synthetic construct
QVQLQESGGGLVQPGGSLRLSCAASGFTLDYYAIGWFRQAPGKEREGVSCISSGDGNTYYADSVKGRFTISRDNAKNTVYLQMNSLKPEDTAVYYCATACGGATGPYDYWGQGTQVTVSS

SEQ ID NO: 132 - protein, 122 aa, synthetic construct
QVQLQESGGGLVQPGGSLRLSCAASGFTLDYYAIGWFRQAPGKEREGVSCISSGDGSTYYADSVKGRFTISRDNAKNTVYLQMNSLKPEDTAVYYCATHCGGSSWSNEYDYWGQGTQVTVSS

SEQ ID NO: 133 - protein, 121 aa, synthetic construct
QVQLQESGGGLVQPGGSLRLSCAASGFTLDYYAIGWFRQAPGKEREGVSCISSNDGSTYYADSVKGRFTISRDNAKNTVYLQMNSLKPEDTAVYYCATACADTTQHEYDYWGQGTQVTVSS

SEQ ID NO: 134 - protein, 120 aa, synthetic construct
QVQLQESGGGLVQPGGSLRLSCAASGFTLDYYAIGWFRQAPGKEREGVSCISSSDGGTYYADSVKGRFTISRDNAKNTVYLQMNSLKPEDTAVYYCATACGGATGPYDYWGQGTQVTVSS

SEQ ID NO: 135 - protein, 125 aa, synthetic construct
QVQLQESGGGLVQPGGSLRLSCAASGFTLDYYAIGWFRQAPGKEREGVSCISSSDGSSSDGNTYYADSVKGRFTISRDNAKNTVYLQMNNLKPEDTAVYYCATACVVTTLYEYDYWGQGTQVTVS

SEQ ID NO: 136 - protein, 121 aa, synthetic construct
QVQLQESGGGLVQPGGSLRLSCAASGFTLDYYAIGWFRQAPGKEREGVSCISSSDGSTYYADSVKGRFTISRDNAKNTVYLQMNSLKPEDTAVYYCATACADTTQYEYDYWGQGTQVTVSP

SEQ ID NO: 137 - protein, 120 aa, synthetic construct
QVQLQESGGGLVQPGGSLRLSCAASGFTLDYYAIGWFRQAPGKEREGVSCISSSDGSTYYADSVKGRFTISRDNAKNTVYLQMNSLKPEDTAVYYCATACGGATGPYDYWGQGTQVTVSS

SEQ ID NO: 138 - protein, 121 aa, synthetic construct
QVQLQESGGGLVQPGGSLRLSCAASGFTLDYYAIGWFRQAPGKEREGVSCISSSGGSTYYADSVKGRFTISRDNAKNTVYLQMNMLKPEDTAVYYCATACADTTQYEYDYWGQGTQVTVSS

SEQ ID NO: 139 - protein, 121 aa, synthetic construct
QVQLQESGGGLVQPGGSLRLSCAASGFTLDYYAIGWFRQAPGKEREGVSCISSSGGSTYYADSVKGRFTISRDNAKNTVYLQMNSLKPEDTAVYYCATACASPVIYEYDYWGQGTQVTVSS

SEQ ID NO: 140 - protein, 121 aa, synthetic construct
QVQLQESGGGLVQPGGSLRLSCAASGFTLDYYAIGWFRQAPGKEREGVSCISSSGGSTYYADSVKGRFTISRDNAKNTVYLQMNSLKPEDTAVYYCATDCAGGTSTPYDYWGQGTQVTVSS

SEQ ID NO: 141 - protein, 121 aa, synthetic construct
QVQLQESGGGLVQPGGSLRLSCAASGFTLGYYAIGWFRQAPGKEREGVSCISSSDGSTYYADSVKGRFTISRDNAKNTVYLQMNSLKPEDTAVYYCATACADTTQYEYDYWGQGTQVTVSS

SEQ ID NO: 142 - protein, 122 aa, synthetic construct
QVQLQESGGGLVQPGGSLRLSCAASGFTLGYYAIGWFRQAPGKEREGVSCISSSDGSTYYADSVKGRFTISRDNAKNTVYLQMNSLKPEDTAVYYCATACANWSSLGPYDYWGQGTQVTVSS

SEQ ID NO: 143 - protein, 120 aa, synthetic construct
QVQLQESGGGLVQPGGSLRLSCAASGFTLGYYAIGWFRQAPGKEREGVSCISSSDGSTYYADSVKGRFTISRDNAKNTVYLQMNSLKPEDTGVYYCATACGGATGPYDYWGQGTQVTVSS

SEQ ID NO: 144 - protein, 121 aa, synthetic construct
QVQLQESGGGLVQPGGSLRLSCEGSGFSLDYYAIGWFRQAPGKEREGVSCISSGDGNTYYADSVKGRFTISRDNAKNTVYLQMNSLKPEDTAVYYCATDCVGFGSNWFDYWGQGTQVTVSS

SEQ ID NO: 145 - protein, 120 aa, synthetic construct
QVQLQESGGGLVQPGGSLRLSCTASGFSLDYYAIGWFRQAPGKEREGVACISSRTGSTYYADSVKGRFTISRDNAKNTVALQMNSLKPEDTAVYYCATACVVVGDQNDYWGQGTQVTVSS

SEQ ID NO: 146 - protein, 120 aa, synthetic construct
QVQLQESGGGLVQPGGSLRLSCTASGFSLDYYAIGWFRQAPGKEREGVSCISSRTGGTYYADSVKGRFTISRDDAKNTVYLQMNSLKPEDTAVYYCATACVVVGDRNDYWGQGTQVTVSS

SEQ ID NO: 147 - protein, 121 aa, synthetic construct
QVQLQESGGGLVQPGGSLRLSCTASGFSLDYYAIGWFRQAPGKEREGVSCISSRTGGTYYADSVKGRFTISRDNAKNTVYLQMNSLKPEDTAVYYCATACVDTTQYEYDYWGQGTQVTVSS

SEQ ID NO: 148 - protein, 120 aa, synthetic construct
QVQLQESGGGLVQPGGSLRLSCTASGFSLDYYAIGWFRQAPGKEREGVSCISSRTGGTYYADSVKGRFTISRDNAKNTVYLQMNSLKPEDTAVYYCATACVVVGDQNDYWGQGTQVTVSS

SEQ ID NO: 149 - protein, 120 aa, synthetic construct
QVQLQESGGGLVQPGGSLRLSCTASGFSLDYYAIGWFRQAPGKEREGVSCISSRTGNTYYADSVKGRFTISRDDAKNMVYLQMNSLKPEDTAVYYCATACVVVGDQNDYWGQGTQVTVSS

SEQ ID NO: 150 - protein, 120 aa, synthetic construct
QVQLQESGGGLVQPGGSLRLSCTASGFSLDYYAIGWFRQAPGKEREGVSCISSRTGSTYYADSVKGRFTISRDDAKNTVYLQMNSLKPEDTAVYYCATACVVVGDQNDYWGQGTQVTVSS

SEQ ID NO: 151 - protein, 120 aa, synthetic construct
QVQLQESGGGLVQPGGSLRLSCTASGFSLGYYAIGWFRQALGKEREGVSCISSRTGSTYYADSVKGRFTVSRDDAKNTVYLQMNSLKPEDTAVYYCATACVVVGDQNDYWGQGTQVTVSS

SEQ ID NO: 152 - protein, 120 aa, synthetic construct
QVQLQESGGGLVQPGGSLRLSCTASGFSLGYYAIGWFRQAPGKEREGVSCISSRTGSTYYADSVKGRFAISRDDAKNTVYLQMNSLKPEDTAVYYCATACVVVGDQNDYWGQGTQVTVSS

SEQ ID NO: 153 - protein, 120 aa, synthetic construct
QVQLQESGGGLVQPGGSLRLSCTASGFSLGYYAIGWFRQAPGKEREGVSCISSRTGSTYYADSVKGRFTISRDDAKNTVYLQMNSLKPEDTAVYYCATACVVVGDQNDYWGQGTQVTVSS

SEQ ID NO: 154 - protein, 120 aa, synthetic construct
QVQLQESGGGLVQPGGSLRLSCTASGFSLGYYAIGWFRQAPGKEREGVSCISSRTGSTYYADSVKGRFTVSRDDAKNTVYLQMNSLKPEDTAVYYCATACVVVGDQNDYWGQGTQVTVSS

SEQ ID NO: 155 - protein, 120 aa, synthetic construct
QVQLQESGGGLVQPGGSLRLSCVASGFPLDYYAIGWFRQAPGKEREGVSCISSSDGSTYYADSVKGRFTISRDNAKNTVYLQMNSLKPEDTAVYYCATACGGATGPYDYWGQGTQVTVSS

SEQ ID NO: 156 - protein, 121 aa, synthetic construct
QVQLQESGGGLVQPGGSLRLSCVASGFSLDYYAIGWFRQAPGKEREGVSCISNSDGSTYYADSVKGRFTISRDNAKNTVYLQMNSLKPEDTAVYYCATACADTTQYAYDYWGQGTQVTVSS

SEQ ID NO: 157 - protein, 121 aa, synthetic construct
QVQLQESGGGLVQPGGSLRLSCVASGFTLDYYAIGWFRQAPGKEREGVSCISSGSDGNTYYADSVKGRFTISRDNAKNTVYLQMNSLKPEDTAVYYCATACSGLTHEYDYWGQGTQVTVSS

SEQ ID NO: 158 - protein, 121 aa, synthetic construct
QVQLQESGGGLVQPGGSLRLSCVASGFTLDYYAIGWFRQAPGKEREGVSCISSSDDSTYYADSVKGRFTISRDNAKNTVYLQMNSLKPEDTAVYYCATACADTTQYEYDYWGQGTQVTVSS

SEQ ID NO: 159 - protein, 121 aa, synthetic construct
QVQLQESGGGLVQPGGSLRLSCVASGFTLDYYAIGWFRQAPGKEREGVSCISSSSDGNTYYADSVKGRFTISRDNAKNTVYLQMNSLKPEDTAVYYCATTCSGLTHEYDYWGQGTQVTVSS

SEQ ID NO: 160 - protein, 121 aa, synthetic construct
QVQLQESGGGLVQPGGSLRLSCVASGFTLGYYAIGWFRQAPGKEREGVSCISSSDGSTYYADSVKGRFTISRDNAKNTVYLQMNSLKPEDTAVYYCATACADTTQYDYDYWGQGTQVTVSS

SEQ ID NO: 161 - protein, 120 aa, synthetic construct
QVQLQESGGGLVQPGGSLRLSCVGSGFTLDYYAIGWFRQAPGKEREGVSCISSNDGSTYYADSVKGRFTISRDNAKNTVYLQMNSLKPEDTAVYYCATACGGATGPYDYWGQGTQVTVSS

SEQ ID NO: 162 - protein, 121 aa, synthetic construct
QVQLQESGGGLVQSGGSLRLSCAASGFPLAYYAIGWFRQAPGKEREGVSCISASDGSTYYADSVKGRFTISRDNAKNTVYLQMNSLKPEDTAVYYCATACAETTLYEYDYWGQGTQVTVSS

SEQ ID NO: 163 - protein, 120 aa, synthetic construct
QVQLQESGGGLVQTGGSLRLSCAASGFTLDYYAIGWFRQAPGKEREGVSCISSSDGSTYYADSVKGRFTISRDNAKNTVYLQMNSLKPEDTAVYYCATACGGATGPYDYWGQGTQVTVSS

SEQ ID NO: 164 - protein, 121 aa, synthetic construct
QVQLQESGGGMVQAGESLRLSCAASGFPLAYYAIGWFRQAPGKEREGVSCISSSDGNTYYADSVKGRFTISRDNAKNTVYLQMNSLKPEDTAVYYCATACADATQHEYDYWGQGTQVTVSS

SEQ ID NO: 165 - protein, 121 aa, synthetic construct
QVQLQESGGGSVQPGESLRLSCAASGFPLDYYAIGWFRQAPGKEREGVSCISASDGSTYYADSVKGRFTISRDNAKNTVYLQMNSLKPEDTAVYYCATACADTTLYEYDYWGQGTQVTVSS

SEQ ID NO: 166 - protein, 121 aa, synthetic construct
QVQLQESGGGSVQPGGSLRLSCAASGFTLDYYAIGWFRQAPGKEREGVSCISSGDGSTYYADSVKGRFTISRDNAKNTVYLQMNSLKPEDTAVYYCATACADTTHYEYDYWGQGTQVTVSS

SEQ ID NO: 167 - protein, 120 aa, synthetic construct
QVQLQESGGGSVQSGGSLRLSCTASGFSLGYYAIGWFRQAPGKEREGVSCISSRTGSTYYADSVKGRFTVSRDDAKNTVYLQMNSLKPEDTAVYYCATACVVVGDQNDYWGQGTQVTVSS

SEQ ID NO: 168 - protein, 121 aa, synthetic construct
QVQLQESGGGSVRPGGSLRLSCAASGFPLAYYAIGWFRQAPGKEREGVSCISSSDGNTYYADAVKGRFTISRDNAKNAVYLQMNSLKPEDTAVYYCATACADTTQHEYDYWGQGTQVTVSS

SEQ ID NO: 169 - protein, 121 aa, synthetic construct
QVQLQESGGGVAQPGGSLRLSCAASGFPLDYYAIGWFRQAPGKEREGVSCISASDGSTYYADSVKGRFTISRDNAKNTVYLQMNSLKPEDTAVYYCATACADTTLYEYDYWGQGTQVTVSS

SEQ ID NO: 170 - protein, 114 aa, synthetic construct
QVQLQESGGGVVQAGGSLKLSCAASGSDFRADAMGWYRQAPGKEREPVAIDSITSIYYVDSVEGRFTISRDNTKNTVYLQMTSLKPEDTAVYYCYARYSGRTYWGRGTQVTVSS

SEQ ID NO: 171 - protein, 122 aa, synthetic construct
QVQLQESGGGVVQPGGSLRLSCAASGFSLDYYAIGWFRQAPGKEREGVSCISSGDGSTYYADSVKGRFTISRDNAKNTVYLQMNSLKPEDTAVYYCATHCGGTSWGTSYDYWGQGTQVTVSS

SEQ ID NO: 172 - protein, 124 aa, synthetic construct
QVQLQESGGGVVQPGGSLRLSCAASGLTFRSVGMGWFRRAPGKEREFVATASPSGVITYYADSVKGRFTISRDNAKNTVYLEMNSLKPEDTAVYYCAVRIYSGSFDNTLAYDYWGQGTQVTVSS

SEQ ID NO: 173 - protein, 120 aa, synthetic construct
QVQLQESGGGVVQPGGSLRLSCTASGFSLGYYAIGWFRQAPGKEREGVSCISSRTGSTYYADSVKGRFTVSRDDAKNTVYLQMNSLKPEDTAVYYCATACVVVGDQNDYWGQGTQVTVSS

SEQ ID NO: 174 - protein, 120 aa, synthetic construct
QVQLQESGGGVVQSGGSLRLSCTASGFSLDYYAIGWFRQAPGKEREGVSCISSRTGSTYYADSVKGRFTISRDDAKNTVYLQMNSLKPEDTAVYYCATACVVVGDQNDYWGQGTQVTVSS

SEQ ID NO: 175 - protein, 115 aa, synthetic construct
QVQLVQSGAEVKKPGASVKVSCKASGYTFTDYEMHWVRQAPGQGLEWMGALDPKTGDTAYSQKFKGKATLTADKSTSTAYMELSSLTSEDTAVYYCTRFYSYTYWGQGTLVTVSS

SEQ ID NO: 176 - protein, 112 aa, synthetic construct
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIK

SEQ ID NO: 177 - protein, 242 aa, synthetic construct
QVQLVQSGAEVKKPGASVKVSCKASGYTFTDYEMHWVRQAPGQGLEWMGALDPKTGDTAYSQKFKGKATLTADKSTSTAYMELSSLTSEDTAVYYCTRFYSYTYWGQGTLVTVSSGGGGSGGGGSGGGGSDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIK

SEQ ID NO: 178 - protein, 240 aa, synthetic construct
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIK

SEQ ID NO: 179 - protein, 257 aa, synthetic construct
MWLQSLLLLGTVACSISEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIK

SEQ ID NO: 180 - protein, 116 aa, synthetic construct
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTV

SEQ ID NO: 181 - protein, 118 aa, synthetic construct
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSS

SEQ ID NO: 182 - protein, 107 aa, synthetic construct
DIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIK

SEQ ID NO: 183 - protein, 130 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVE

SEQ ID NO: 184 - protein, 107 aa, synthetic construct [sequence omitted]

SEQ ID NO: 185 - protein, 108 aa, synthetic construct
LVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTV

SEQ ID NO: 186 - protein, 120 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 187 - protein, 255 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESSGGGGSGGGGSGGGGSLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTV

SEQ ID NO: 188 - protein, 244 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 189 - protein, 244 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRGGGGSGGGGSGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 190 - protein, 244 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKR

SEQ ID NO: 191 - protein, 121 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 192 - protein, 108 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKR

SEQ ID NO: 193 - protein, 191 aa, synthetic construct
MEFGLSWLFLVAILKGVQCGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 194 - protein, 247 aa, synthetic construct
QVQLVQSGAEVKKPGASVKVSCKASGYTFTNYGMNWVRQAPGQGLEWMGWINTYTGEPTYADAFKGRVTMTTDTSTSTAYMELRSLRSDDTAVYYCARDYGDYGMDYWGQGTTVTVSSGSTSGSGKPGSSEGSTKGDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 195 - protein, 247 aa, synthetic construct
QVQLQESGPGLVKPSQTLSLTCTVSGGSISSGYNWHWIRQPPGKGLEWIGYIHYTGSTNYNPALRSRVTISVDTSKNQFSLKLSSVTAADTAVYYCARIYNGNSFPYWGQGTTVTVSSGGGGSGGGGSGGGGSDIVMTQSPDSLAYSLGERATINCKSSQSLFNSGNQKNYLTWYQQKPGQPPKLLIYWASTRESGVPDRFSGSGSGTDIFITISSLQAEDVAVYYCQNAYSFPYTFGGGTKLEIKR

SEQ ID NO: 196 - protein, 19 aa, synthetic construct
LIRMAVAGLVLVALLAILV

SEQ ID NO: 197 - protein, 71 aa, synthetic construct
DSIHQDYTTQNLIRMAVAGLVLVALLAILVENWHSHTALNKEASADVAEPSWSQQMCQPGLTFARTPSVCK

SEQ ID NO: 198 - protein, 28 aa, synthetic construct
IHQDYTTQNLIRMAVAGLVLVALLAILV

SEQ ID NO: 199 - protein, 23 aa, synthetic construct
SSSPMGIIVAVVTGIAVAAIVAA

SEQ ID NO: 200 - protein, 20 aa, synthetic construct
SFCLVMVLLFAVDTGLYFSV

SEQ ID NO: 201 - protein, 37 aa, synthetic construct
GLAVSTISSFFPPGYQVSFCLVMVLLFAVDTGLYFSV

SEQ ID NO: 202 - protein, 21 aa, synthetic construct
IYIWAPLAGTCGVLLLSLVIT

SEQ ID NO: 203 - protein, 24 aa, synthetic construct
IYIWAPLAGTCGVLLLSLVITLYC

SEQ ID NO: 204 - protein, 26 aa, synthetic construct
IYLIIGICGGGSLLMVFVALLVFYIT

SEQ ID NO: 205 - protein, 27 aa, synthetic construct
FWVLVVVGGVLACYSLLVTVAFIIFWV

SEQ ID NO: 206 - protein, 25 aa, synthetic construct
ILLPLIIGLILLGLLALVLIAFCII

SEQ ID NO: 207 - protein, 86 aa, synthetic construct
ALSNSIMYFSHFVPVFLPAKPTTTPAPRPPTPAPTIASQPLSLRPEACRPAAGGAVHTRGLDIYIWAPLAGTCGVLLLSLVITLYC

SEQ ID NO: 208 - protein, 83 aa, synthetic construct
ALSNSIMYFSHFVPVFLPAKPTTTPAPRPPTPAPTIASQPLSLRPEACRPAAGGAVHTRGLDIYIWAPLAGTCGVLLLSLVIT

SEQ ID NO: 209 - protein, 46 aa, synthetic construct
LYCRRLKIQVRKAAITSYEKSDGVYTGLSTRNQETYETLKHEKPPQ

SEQ ID NO: 210 - protein, 45 aa, synthetic construct
LYCRLKIQVRKAAITSYEKSDGVYTGLSTRNQETYETLKHEKPPQ

SEQ ID NO: 211 - protein, 42 aa, synthetic construct
RLKIQVRKAAITSYEKSDGVYTGLSTRNQETYETLKHEKPPQ

SEQ ID NO: 212 - protein, 41 aa, synthetic construct
ENWHSHTALNKEASADVAEPSWSQQMCQPGLTFARTPSVCK

SEQ ID NO: 213 - protein, 25 aa, synthetic construct
KTNIRSSTRDWKDHKFKWRKDPQDK

SEQ ID NO: 214 - protein, 35 aa, synthetic construct
YEDMRGILYAAPQLRSIRGQPGPNHEEDADSYENM

SEQ ID NO: 215 - protein, 62 aa, synthetic construct
KKVAKKPTNKAPHPKQEPQEINFPDDLPGSNTAAPVQETLHGCQPVTQEDGKESRISVQERQ

SEQ ID NO: 216 - protein, 219 aa, synthetic construct
QRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 217 - protein, 171 aa, synthetic construct
PLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 218 - protein, 211 aa, synthetic construct
MSNGYSTDENFRYLISCFRARVKMYIQVEPVLDYLTFLPAEVKEQIQRTVATSGNMQAVELLLSTLEKGVWHLGWTREFVEALRRTGSPLAARYMNPELTDLPSPSFENAHDEYLQLLNLLQPTLVDKLLVRDVLDKCMEEELLTIEDRNRIAAAENNGNESGVRELLKRIVQKENWFSAFLNVLRQTGNNELVQELTGSDCSESNAEIEN

SEQ ID NO: 219 - protein, 17 aa, synthetic construct
MWLQSLLLLGTVACSIS

SEQ ID NO: 220 - protein, 4 aa, synthetic construct
SGGG

SEQ ID NO: 221 - protein, 4 aa, synthetic construct
GSGS

SEQ ID NO: 222 - protein, 6 aa, synthetic construct
SGSGSG

SEQ ID NO: 223 - protein, 7 aa, synthetic construct
SGGGGSG

SEQ ID NO: 224 - protein, 9 aa, synthetic construct
SSSGGGGSG

SEQ ID NO: 225 - protein, 15 aa, synthetic construct
GGGGSGGGGSGGGGS

SEQ ID NO: 226 - protein, 14 aa, synthetic construct
GGGGSGGGGSGGGG

SEQ ID NO: 227 - protein, 186 aa, synthetic construct
APITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTD

SEQ ID NO: 228 - protein, 186 aa, synthetic construct
APITAYAQQTRGLLGCIITSLTGRDKNQVEGEVQIMSTATQTFLATCINGVCWTVYHGAGTRTIASPKGPVIQMYTNVDQDLVGWPAPQGSRSLTPCTCGSSDLYLVTRHADVIPVRRRGDGRGSLLSPRPISYLKGSSGGPLLCPAGHAVGLFRAAVCTRGVAKAVDFIPVENLETTMRSPVFTD

SEQ ID NO: 229 - protein, 186 aa, synthetic construct; identical to SEQ ID NO: 227

SEQ ID NO: 230 - protein, 186 aa, synthetic construct
APITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTD

SEQ ID NO: 231 - protein, 180 aa, synthetic construct
APITAYAQQTRGLLGCIITSLTGRDKNQVEGEVQIVSTATQTFLATCINGVCWTVYHGAGTRTIASPKGPVIQMYTNYDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMR

SEQ ID NO: 232 - protein, 9 aa, synthetic construct
DEMEECSQH

SEQ ID NO: 233 - protein, 10 aa, synthetic construct
DEMEECSQHL

SEQ ID NO: 234 - protein, 9 aa, synthetic construct
EDVVPCSMG

SEQ ID NO: 235 - protein, 10 aa, synthetic construct
EDVVPCSMGS

SEQ ID NO: 236 - protein, 11 aa, synthetic construct
ECTTPCSGSWL

SEQ ID NO: 237 - protein, 10 aa, synthetic construct
SSPPAVTLTH

SEQ ID NO: 238 - protein, 14 aa, synthetic construct
TGCVVIVGRIVLSG

SEQ ID NO: 239 - protein, 185 aa, synthetic construct
MGKPLHPNDKVMGPGVSYLVRYMGCVEVLQSMRALDFNTRTQVTREAISLVCEAVPGAKGATRRRKPCSRPLSSILGRSNLKFAGMPITLTVSTSSLNLMAADCKQIIANHHMQSISFASGGDPDTAEYVAYVAKDPVNQRACHILECPEGLAQDVISTIGQAFELRFKQYLRDIEQVPQQPTLK

SEQ ID NO: 240 - protein, 7 aa, synthetic construct
MLAPYIP

SEQ ID NO: 241 - protein, 186 aa, synthetic construct
MGKPLHPNDKVMGPGVSYLVRYMGCVEVLQSMRALDFNTRTQVTREAISLVCEAVPGAKGATRRRKPCSRPLSSILGRSNLKFAGMPITLTVSTSSLNLMAADCKQIIANHHMQSISFASGMLAPYIPEYVAYVAKDPVNQRACHILECPEGLAQDVISTIGQAFELRFKQYLRDIEQVPQQPTLK

SEQ ID NO: 242 - protein, 1151 aa, synthetic construct
MWLQSLLLLGTVACSISQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRSGGGGAAAGSDSIHQDYTTQNLIRMAVAGLVLVALLAILVENWHSHTALNKEASADVAEPSWSQQMCQPGLTFARTPSVCKGGGSGGGDEMEECSQHGGGSGGGMALAPERAAPRVLFGEWLLGEISSGCYEGLQWLDEARTCFRVPWKHFARKDLSEADARIFKAWAVARGRWPPSSRGGGPPPEAETAERAGWKTNFRCALRSTRRFVMLRDNSGDPADPHKVYALSRELCWREGPGTDQTEAEAPAAVPPPQGGPPGPFLAHTHAGLQAPGPLPAPAGDKGDLLLQAVQQSCLADHLLTASWGADPVPTKAPGEGQEGLPLTGACAGGPGLPAGELYGWAVETTPSPEGVSSLDSSSLSLCLSSANSLYDDIECFLMELEQPAGSGEGRGSLLTCGDVEENPGPGSGASSGMADSANHLPFFFGNITREEAEDYLVQGGMSDGLYLLRQSRNYLGGFALSVAHGRKAHHYTIERELNGTYAIAGGRTHASPADLCHYHSQESDGLVCLLKKPFNRPQGVQPKTGPFEDLKENLIREYVKQTWNLQGQALEQAIISQKPQLEKLIATTAHEKMPWFHGKISREESEQIVLIGSKTNGKFLIRARDNNGSYALCLLHEGKVLHYRIDKDKTGKLSIPEGKKFDTLWQLVEHYSYKADGLLRVLTVPCGGGSGGGTGCVVIVGRIVLSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTH

SEQ ID NO: 243 - protein, 10 aa, synthetic construct
SGGGGAAAGS

SEQ ID NO: 244 - protein, 7 aa, synthetic construct
GGGSGGG

SEQ ID NO: 245 - protein, 285 aa, synthetic construct
MALAPERAAPRVLFGEWLLGEISSGCYEGLQWLDEARTCFRVPWKHFARKDLSEADARIFKAWAVARGRWPPSSRGGGPPPEAETAERAGWKTNFRCALRSTRRFVMLRDNSGDPADPHKVYALSRELCWREGPGTDQTEAEAPAAVPPPQGGPPGPFLAHTHAGLQAPGPLPAPAGDKGDLLLQAVQQSCLADHLLTASWGADPVPTKAPGEGQEGLPLTGACAGGPGLPAGELYGWAVETTPSPEGVSSLDSSSLSLCLSSANSLYDDIECFLMELEQPAGSG

SEQ ID NO: 246 - protein, 18 aa, synthetic construct
EGRGSLLTCGDVEENPGP

SEQ ID NO: 247 - protein, 258 aa, synthetic construct
ASSGMADSANHLPFFFGNITREEAEDYLVQGGMSDGLYLLRQSRNYLGGFALSVAHGRKAHHYTIERELNGTYAIAGGRTHASPADLCHYHSQESDGLVCLLKKPFNRPQGVQPKTGPFEDLKENLIREYVKQTWNLQGQALEQAIISQKPQLEKLIATTAHEKMPWFHGKISREESEQIVLIGSKTNGKFLIRARDNNGSYALCLLHEGKVLHYRIDKDKTGKLSIPEGKKFDTLWQLVEHYSYKADGLLRVLTVPC

SEQ ID NO: 248 - protein, 215 aa, synthetic construct
TGCVVIVGRIVLSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTH